(19) United States
(12) Patent Application Publication
(10) Pub. No.: US 2023/0355740 A1
(43) Pub. Date: Nov. 9, 2023

(54) COMPOSITIONS AND METHODS OF USE THEREOF FOR PREVENTION AND TREATMENT OF INFLUENZA INFECTIONS

(71) Applicants: University of Georgia Research Foundation, Inc., Athens, GA (US); THE UNITED STATES OF AMERICA, AS REPRESENTED BY THE SECRETARY OF AGRICULTURE, Washington, DC (US)

(72) Inventors: Amy L. Vincent, Nevada, IA (US); Marcus E. Kehrli, Ames, IA (US); Daniel R. Perez, Athens, GA (US); Daniela Rajao, Athens, GA (US)

(21) Appl. No.: 18/245,115

(22) PCT Filed: Sep. 13, 2021
(86) PCT No.: PCT/US2021/050093

§ 371 Date: Mar. 13, 2023

Related U.S. Application Data
(60) Provisional application No. 63/077,454, filed on Sep. 11, 2020.

(51) Int. Cl. A61K 39/145; C07K 14/005; C12N 7/00; C12N 15/86

(52) U.S. Cl. CPC A61K 39/145; C07K 14/005; C12N 7/00; C12N 15/86; A61K 2039/5254

(57) ABSTRACT

Recombinant constructs, influenza viral genomes including the recombinant constructs, influenza viruses including the constructs, and vaccine formulations formed thereof for inducing or increasing an immune response against influenza virus are provided. The compositions typically include a nucleic acid having a nucleic acid sequence encoding IgA-inducing protein (IGIP) polypeptide that can positively regulate IgA expression operably linked to expression of a hemagglutinin or a neuraminidase. When the nucleic acid is expressed by recombinant influenza virus in infected cells, it preferably enhances IgA production against influenza virus. Live attenuated virus expressing IGIP, and methods of use thereof for treating and preventing influenza infections are also provided.

### CROSS-REFERENCE TO RELATED APPLICATIONS

This application claims benefit of and priority to U.S. Provisional Application No. 63/077,454, filed Sep. 11, 2020, which is hereby incorporated herein by reference in its entirety.

### STATEMENT REGARDING FEDERALLY SPONSORED RESEARCH OR DEVELOPMENT

This invention was made with government support under HHSN272201400008C and AI146448 awarded by the National Institutes of Health and 58-5030-5-047 by the U.S. Department of Agriculture. The government has certain rights in the invention.

### REFERENCE TO SEQUENCE LISTING

The Sequence Listing submitted as a text file named “UGA_2019_094_02_PCT_ST25.txt,” created on Sep. 13, 2021, and having a size of 11,269 bytes is hereby incorporated by reference pursuant to 37 C.F.R § 1.52(e)(5).

### FIELD OF THE INVENTION

This invention is generally in the field of recombinant influenza viruses and their use in vaccine formulations and protocols.

### BACKGROUND OF THE INVENTION

Influenza, commonly known as the flu, is an acute respiratory illness caused by influenza viruses. Influenza virus infections continue to pose a major public health threat worldwide associated with seasonal epidemics and sporadic pandemics. Most people recover from fever and other symptoms within a week without requiring medical attention. However, influenza can cause severe illness or death in high-risk groups. For example, Influenza A (IAV) generates 3-5 million cases of severe disease, and between 300,000 and 600,000 deaths worldwide annually (Girard, et al., Vaccine, 23, 5708-5724 (2005), Iuliano, et al., Lancet, 391, 1285-1300 (2018)). For the U.S., influenza virus infections result in an average economic impact of $87 billion due to prophylactic, therapeutic and hospitalization costs, and missed school or workdays (Molinari, et al., Vaccine, 25, 5086-5096 (2007), Glezen, et al., Am. J. Public Health, 103, e43-e51 (2013), Gasparini, et al., Hum. Vaccines Immunother, 8, 21-28 (2012)).

Vaccination is considered the first line of defense against influenza (Erbelding, J Infect Dis, 218(3):347-354 (2018)) and though not 100 percent effective, is still the best way to prevent infection. Currently, the seasonal flu vaccines only target several (usually three or four) strains that were predicted to be the most common in the upcoming season, however, the ever-changing nature of these viruses make vaccines ineffective after a single season or against pandemic strains. The FDA has approved three types of influenza virus vaccines for human use: split virion or subunit inactivated influenza virus (IIV), recombinant influenza protein (RIV), and live attenuated influenza virus (LAIV) vaccines. The IIV and RIV vaccines can elicit the production of antibodies that target epitopes on the HA yet produce limited or no cellular immunity. In contrast, LAIV can elicit a combination of humoral and cellular responses by mimicking a natural infection (Lopez, et al., Vaccines, 8, 434 (2020), Yamayoshi & Kawaoka, Nat. Med., 25, 212-220 (2019)). Despite the inherent ability of LAIVs. to provide immunity against multiple viral targets, they are not suitable for everyone due to safety concerns in immunologically compromised individuals (Yamayoshi & Kawaoka, Nat. Med., 25, 212-220 (2019), Grohskopf, et al., MMWR Recomm Rep., 69, 1-24 (2020). Thus, improving the safety profile of LAIVs. while maintaining efficacy is desirable to expand the use of such vaccines in the population.

Upon vaccination, the body produces specific antibodies against the viruses that are present in the vaccine. IgG and IgA are two major isotypes of antibodies that the body produces to protect against influenza infection. IgG is the major isotype found in blood circulation, and IgA is the predominant isotype found in mucosal tissues, including the airway. IgA responses are considered of great significance to prevent and/or control a myriad of genital, intestinal, and respiratory infections, including influenza (Corthesy, Front. Immunol., 4, 185 (2013)). As influenza viruses enter the host via mucosal surfaces, both IgA and IgG responses are detected at the airway mucosa with neutralizing activity against influenza, however, an abundance of IgA is important in providing the first line of protection. IgA, particularly secretory IgA in its multiple multimeric forms, is typically more broadly neutralizing than IgG (Suzuki, et al., Proc. Natl. Acad. Sci. USA, 112, 7809-7814 (2015)). IgA neutralizes pathogens without causing inflammation because of its inability to fix and activate the complement cascade (Cerutti, Nat. Rev. Immunol., 8, 421-434 (2008)).

Additionally, the IgA antibody shows remarkable higher cross-binding activities than the IgG antibody, meaning the IgA antibody can even target a broader-spectrum of viruses which are not included in the vaccines. IgA, particularly secretory IgA (sIgA) in its multiple multimeric forms, is typically more broadly-neutralizing than IgG6. IgA neutralizes pathogens without causing inflammation because of its inability to fix and activate the complement cascade (Cerutti, et al., Nat Rev Immunol, 8(6):421-34 (2008)). In contrast, IgG responses that bind, but do not neutralize the virus (non-protective antibodies), have been linked to immune complex-mediated disease in middle-age people that were severely ill after infection with the pandemic HIN1 virus (H1N1pdm) in 2009 or the pandemic H2N2 virus in 1957 (Monsalvo, et al., Nature medicine, 17(2):195-9 (2011)).

The effectiveness of current influenza vaccines is still very low, ranging from 10% to 60%. The intranasal flu vaccine is considered even less effective than flu shots. The influenza vaccines available to date have not been designed to specifically upregulate IgA responses and/or tilt the IgA/IgG balance towards more prominent IgA responses, particularly within the confinements of the airway (Boyaka, et al., J Immunol, 199(1):9-16 (2017)). Currently approved or experimental live attenuated influenza virus vaccines are thought to stimulate mucosal and systemic IgA and IgG as well as T-cell mediated immunity because they more closely resemble a natural virus infection, but their relative influence on promoting IgA responses is not well understood.

Thus, there remains a need for improved vaccine strategies for protection from, and treatment of, influenza.

It is therefore an object of the invention to provide improved compositions and methods of use thereof for protection from, and treatment of, influenza viral infection.

It is a further object of the invention to provide compositions and methods of use thereof for improving mucosal IgA responses against influenza.

It is a further object of the invention to provide compositions and methods of use thereof that enhance and/or broaden protection against antigenic drifted variants or clades while minimizing the risks associated with suboptimal antibody-antigen match, disease enhancement, and immunopathology.

### SUMMARY OF THE INVENTION

Recombinant constructs, viral genomes including the recombinant constructs, viruses including the constructs, and vaccine formulations formed thereof for inducing or increasing an immune response against the virus are provided. The compositions typically include a nucleic acid having a nucleic acid sequence encoding IgA-inducing protein (IGIP) polypeptide, optionally that can positively regulate IgA expression. When the nucleic acid is expressed by recombinant virus in infected cells, it preferably enhances IgA production against the virus. In preferred embodiments, the recombinant virus is an influenza virus and the immune response against influenza.

For example, nucleic acids including a nucleic acid sequence encoding an IGIP polypeptide that can induce or increase IgA expression and a nucleic acid sequence encoding a hemagglutinin (abbreviated interchangeably herein as H and HA) or a neuraminidase (abbreviated interchangeably herein as N and NA), operably linked to one or more expression control sequences are provided.

The IGIP can be a mature form of a naturally occurring IGIP protein e.g., from bovine, human, mouse, or another animal. In some embodiments, the IGIP polypeptide includes the mature form of the IGIP of any one of SEQ ID NOS:1-12, or a functional fragment thereof, or variant thereof with at least 70%, 75%, 80%, 85%, 90%, 95%, or more sequence identity to any one of SEQ ID NOS:1-12. The nucleic acid can include a sequence encoding the autologous IGIP signal peptide sequence or a heterologous signal peptide sequence operably linked to the sequence encoding the IGIP.

In some embodiments, the H is a mature form of an H from an influenza Type A virus, such as any one of H1 to H18 subtypes. In some embodiments, the H is a mature form of an H from an influenza Type B virus. The nucleic acid sequence encoding the H can further include a nucleic acid sequence encoding the autologous H signal peptide sequence or a heterologous signal peptide sequence operably linked to the sequence encoding the H.

In some embodiments, the N is a mature form of an N from an influenza Type A virus, such as any one of N1 to N11 subtypes. In some embodiments, the N is a mature form of an N from an influenza Type B virus. The nucleic acid sequence encoding the N can further include a nucleic acid sequence encoding the autologous N signal peptide sequence or a heterologous signal peptide sequence operably linked to the sequence encoding the N.

The nucleic acid can also include or encode additional elements. For example, the sequence encoding the IGIP polypeptide and the sequence encoding H or N can be separated by a nucleic acid sequence encoding a protease cleavage site, optionally wherein the protease cleavage site is a Furin cleavage site. Additionally or alternatively, the sequence encoding the IGIP polypeptide and the sequence encoding H or N can be separated by a nucleic acid sequence encoding a self-cleaving peptide, optionally wherein the self-cleaving peptide is 2A self-cleaving peptide. The sequence encoding the IGIP polypeptide and the sequence encoding H or N can also be separated by a nucleic acid sequence encoding a peptide linker, optionally wherein the peptide linker includes one or more glycines and/or one or more serines.

In some embodiments, the nucleic acid is a single stranded negative sense RNA genomic segment of an influenza virus. Thus, in some embodiments, the orientation of the nucleic acid sequences is: 5′ nucleic acid sequence encoding the IGIP polypeptide-nucleic acid sequence encoding H or N 3′. In other embodiments, the orientation of the nucleic acid sequences is: 5′ nucleic acid sequence encoding H or N-nucleic acid sequence encoding the IGIP polypeptide 3′. Reverse orientations, reverse complements, and double stranded nucleic acid sequences corresponding to the single stranded negative sense RNA genomic segments are also provided.

In some embodiments, the nucleic acid further includes a 5′ untranslated region (UTR), a 3′ untranslated region (UTR), or a combination thereof. The 5′ UTR, 3′ UTR, or combination thereof can be from an influenza virus. In some embodiments, the nucleic acid encodes H and includes the 5′ UTR and 3′ UTR of genome Segment 4 of an influenza virus at the 5′ and 3′ ends respectively of the nucleic acid. In some embodiments, the nucleic acid encodes N and includes the 5′ UTR and 3′ UTR of genome Segment 6 of an influenza virus at the 5′ and 3′ ends respectively of the nucleic acid.

The nucleic acids can be formed of, for example, DNA or RNA, can be single stranded or double stranded, and can be linear or circular. The reverse complements to the nucleic acids are also expressly disclosed. Vectors and viral genomic segments including the nucleic acid and/or its reverse complement are also provided.

Recombinant viruses having a genome encoding the disclosed nucleic acids are also provided. In some embodiments, the disclosed nucleic acids form part of a recombinant influenza virus genome segment, and the remaining viral genomic segments are from an influenza virus. Thus, the recombinant virus can be, and most typically is, a recombinant influenza virus. The influenza virus can be a Type A or a Type B influenza virus. For example, in some embodiments, the genomic structure of a recombinant Type A influenza virus is: Segment 1 encoding PB2; Segment 2 encoding PB1 and optionally PB1-F2; Segment 3 encoding PA and optionally PA-X; Segment 4 encoding H; Segment 5 encoding NP; Segment 6 encoding N; Segment 7 encoding M1 and M2; and Segment 8 encoding NS1 and NEP; wherein Segment 4 includes a nucleic acid sequence encoding an IGIP polypeptide operably linked to a nucleic acid sequence encoding H or Segment 6 includes a nucleic acid sequence encoding an IGIP polypeptide operably linked to a nucleic acid sequence encoding N.

In some embodiments, the genomic structure of a recombinant Type B influenza virus is: Segment 1 encoding PB1; Segment 2 encoding PB2; Segment 3 encoding PA; Segment 4 encoding H; Segment 5 encoding NP; Segment 6 encoding N and NB; Segment 7 encoding M1 and BM2; and Segment 8 encoding NS1 and NEP; wherein Segment 4 includes a nucleic acid sequence encoding an IGIP polypeptide operably linked to a nucleic acid sequence encoding H or Segment 6 includes a nucleic acid sequence encoding an IGIP polypeptide operably linked to a nucleic acid sequence encoding N.

Most typically, the influenza virus is formed from an attenuated influenza virus backbone. The attenuated virus can include a reassorted genome, temperature sensitive mutation(s), NS1 truncation, elastase-dependency, rearranged genome, or a combination thereof. Exemplary attenuated viruses include OH/04 att, cold-adapted Leningrad (ca/LEN), or B/Bris att. In some embodiments, the introduction of the sequence encoding the IGIP polypeptide increases or enhances attenuation of the recombinant virus relative to its parent, backbone virus without the IGIP encoding sequence.

Compositions and methods of using other viruses to express IGIP and enhance immune responses against the viruses are also provided. For example, in some embodiments, the virus is a recombinant vaccinia virus, adenovirus, herpes virus, paramyxovirus 5, Newcastle disease virus, measles virus, yellow fever virus, mumps virus, rubella virus, or rotavirus having a genome encoding an IgA-inducing protein (IGIP) polypeptide that can increase IgA expression when expressed in a cell infected by the virus.

Pharmaceutical compositions including live and/or inactivated recombinant virus in a pharmaceutically acceptable carrier for administration to a subject are also provided. In some embodiments, the compositions include one or more additional influenza viruses, for example, one or more additional live attenuated influenza viruses. Thus, the pharmaceutical composition can be a multivalent vaccine formulation. One or more of the viruses may express an IGIP polypeptide. Preferred influenza viruses for vaccine formulations include, but are not limited to, live attenuated H1N1, H2N2, H3N2, H5N1, H7N7, H1N2, H9N2, H7N2, H7N3, H10N7, H7N9, and/or H6N1 influenza A subtypes, and one or more live attenuated influenza B viruses. Any of the pharmaceutical compositions can further include an adjuvant. The pharmaceutical compositions can be formulated for, for example, intradermal or intramuscular injection, or intranasal delivery. In preferred embodiments, the compositions include an effective amount of recombinant virus(es), preferably influenza virus(es), to induce an immune response against a virus, preferably influenza virus, in a subject. The immune response can be, for example, an increase in production of IgA antibodies against influenza virus, preferably in the airway mucosa.

Thus, methods of inducing or increasing an immune response against recombinant virus, including recombinant influenza are also provided. The methods typically include administering to a subject in need thereof an effective amount of the pharmaceutical composition including live recombinant virus(es) to induce or increase an immune response against the influenza virus(es) in the subject. The immune response can provide a preventative or therapeutic effect against infection by influenza virus(es). The pharmaceutical composition can be administered by, for example, injection or intranasal delivery. The subject can be, for example, a human, bird, pig, horse, ferret, whale, seal, dog, cat, and rodent, or other animal capable of harboring influenza virus or an infection thereof. Vaccination regimes are also provided and may include a single administration, or repeating the administration one or more times 1, 2, 3, 4, 5, 6, 7, 8, 9, or 10 days, weeks, or months apart.

In some embodiments, the disclosed compositions and methods of use thereof enhance and/or broaden protection against antigenic drifted variants or clades, reduce the risks associated with suboptimal antibody-antigen match, reduce disease enhancement, reduce immunopathology, or combination thereof relative to alternative influenza vaccination composition and methods.

### BRIEF DESCRIPTION OF THE DRAWINGS

FIG. 1A is an alignment of the predicted IGIP in different mammalian species (Bovine (SEQ ID NO:7), Ferret (SEQ ID NO: 12), Swine (SEQ ID NO:12), Human (SEQ ID NO:8), and Mouse (SEQ ID NO:10)). The mature swine IGIP sequence (SEQ ID NO:4) used in this study is shown (“Flu”). FIGS. 1B and 1C are schematics illustrating strategies to produce type A and B IGIP-LAIV vaccines. Segment 4 (HA) (TA) and 6 (NA) (1B) from type A and type B strains were modified to encode the IGIP peptide in the context of additional sequences to produce its release from the major protein product (intergenic region). FIG. 1B illustrates a polyprotein sequence encoded by a reverse genetics plasmid (5′-3′): HA signal peptide-mature IGIP-G4S linker-Furin Cleavage Site-TvV 2a self-cleaving peptide-Gluc signal peptide-HA (SEQ ID NO:28). FIG. 1C illustrates a polyprotein sequence encoded by a reverse genetics plasmid (5′-3′): NA-Furin Cleavage Site-TvV 2a self-cleaving peptide -Gluc signal peptide-mature IGIP (SEQ ID NO:29). FIG. 1D is a line graph showing the growth kinetics profiles of IGIP-H1att and H1att in MDCK and MDCK STAT1 KO cells. Experiments were performed two times independently, each time in triplicate. Titers were determined by RT-qPCR and expressed as log 10 TCID50 equivalents. Gray area represents the area below the level of detection of the assay. FIGS. 1E-1H are line graphs showing Log₁₀ TCID₅₀/ml of Ty/04 WT, Ty/04 att, and IGIP-HA Ty/04 att at 36° C. (1C), 37° C. (1D), 38° C. (1E), and 41° C. (1F) over time (hours post infection (hpi)). Abbreviations: FP, fusion peptide; TM, transmembrane domain; CT, c-terminal region; G4S, poly-glycine protein linker; Furin CS, furin cleavage site; Tav2A, Thosea assigna virus 2A protein sequence; GlucS, Signal peptide of Gaussia luciferase.

FIG. 2A is a schematic representation of a safety/efficacy vaccine study in DBA/2J mice using a primer-boost vaccination regime. FIG. 2B is schematic providing a more detailed representation of the evaluation of the different viruses in DBA/2 J model.

FIGS. 3A and 3B are line graphs showing weight change (%), and FIGS. 3C and 3D are survival curves showing % survival over time, for male (3A, 3C) and female (3B, 3D) DBA/2J after prime vaccination with influenza A H1N1 LAIV strains. Mice (n=16, ½ female) were intranasally inoculated with 1×10⁵ TCID₅₀/mouse and clinical signs monitored daily. FIGS. 3E and 3F are line graphs weight change (%) and survival curves showing % survival over time, respectively. Mice (n=16) were mock inoculated (PBS; white circles) or inoculated with 1×10⁵ TCID50/mouse of IGIP-H1att (blue circles), H1att (black circles) or H1caLen (grey circles). Weight changes (grey area below the 75% mark the point where mice have reached a humane endpoint) (3E) and survival were monitored for 14 days after virus inoculation (3F).

FIGS. 4A and 4B are line graphs showing weight change (%), and FIGS. 4C and 4D are survival curves showing % survival over time, for male (4A, 4C) and female (4B, 4D) DBA/2J after high dose of H1N1. Mice were intranasally inoculated with 1×10⁶ TCID₅₀/mouse of the mouse adapted A/California/04/09 (10,000 MLD₅₀) and clinical signs monitored daily; mice that reached humane endpoints were humanely euthanized. FIG. 4E-4H plots also showing data on efficacy of IGIP-H1att against H1N1 lethal challenge in DBA/2 J mice. Mice (n=12/group) previously mock-vaccinated were mock challenged (white circles) or challenged (black circles) with 1×10⁶ TCID50/mouse of Ca/04 (H1N1). Mice previously vaccinated with IGIP-H1att (blue circles) or H1caLen (grey circles) were challenged similarly. (4E) Weight changes (Grey area represents mice reaching humane endpoints) and (4F) Survival were monitored for 12 days. At 5 dpc, mice (n=4/group) were humanely euthanized, and the viral load was evaluated in tissue samples from (4G) lungs and (4H) nasal turbinates.

FIGS. 5A and 5B are bar graphs showing macroscopic lung lesions (%, 5A) and microscopic lung lesions (5B), and FIGS. 5C and 5D are bar graphs showing viral titers in bronchoalveolar lavage fluid (5C) or nasal swabs (5D) for pigs after challenge. Pigs were intranasally and intratracheally inoculated with 3×10⁶ TCID₅₀/pig of H1N1 A/swine/Iowa/A01778877/2016 or H3N2 A/swine/Ohio/A01354299/2017.

FIGS. 6A and 6B are line graphs showing weight change (%), and FIGS. 6C and 6D are survival curves showing % survival over time, for male (6A, 6C) and female (6B, 6D) DBA/2J after prime vaccination with influenza B LAIV strains. Mice were intranasally inoculated with 1×10⁶ TCID₅₀/mouse and clinical signs monitored daily.

FIGS. 7A and 7B are line graphs showing weight change (%), and FIGS. 7C and 7D are survival curves showing % survival over time, for male (7A, 7C) and female (7B, 7D) DBA/2J after high dose of IBV. Mice were intranasally inoculated with 1×10⁷ TCID₅₀/mouse of B/Brisbane/60/2008 PB2 F406Y virus (≥10 MLD₅₀) and clinical signs monitored daily; mice that reached humane endpoints were humanely euthanized.

FIG. 8A-8D are plots showing IgG serum responses 20 dpb in IGIP-H1att- and H1caLen-vaccinated mice. Mice (n=4/group) were bled at 20 dpb, and the sera were used to evaluate HAI, VN and antibody reactivity against a panel of influenza antigens printed on a microarray. Samples from IGIP-H1att- and H1caLen-vaccinated mice indicated by blue dots/bars and grey dots/bars, respectively. PBS control samples are shown as white dots/bars. (8A) HAI and VN titers. VN titers were established using a recombinant Ca/04 virus carrying PB1-Nluc and evaluated by two independent methods at 48 hpi using a classical HA assay and Nluc activity. Levels of IgG antibodies against (8B) H1, (8C) H5 and (8D) H9. The reactivity of each serum sample against each antigen is shown by dots/antigen and the results are expressed as the mean of fluorescent intensity (MFI) of each value±SD. The statistically significant differences between IGIP-H1att and caLen are depicted with asterisks. FIGS. 8E-8Q are plots showing Influenza antigen microarray data combined. The combined data including the different antigens was plotted. Serum (8E) IgG and (8F) IgA at 20 dpb against H1. Serum (8G) IgG and (8H) IgA at 14 dpc against H1. (8I-8J) IgG in (8I) NW and (8J) BALF against H1 at 14 dpc. (8K-8L) IgA in (8K) NW and (8L) BALF against H1 at 14 dpc. (8M) Serum IgG against NA. (8N-8O) Serum (8N) IgA and (8O) IgG against internal proteins. (8P-8Q) Mucosal (8P) IgG and (8Q) IgA against internal IAV proteins. Statistically significant differences between IGIP-H1att and caLen marked with asterisks. FIG. 8R-8X are plots showing Influenza antigen microarray data against group 1 HA (H2, H6, H8 and H11) and group 2 HA (H3, H4, H7 and H10). (8R-8S) IgG against (8R) group 1 and (8S) group 2 HAs at 20 dpb. (8T-8U) IgA against (8T) group 1 and (8U) group 2 HAs at 20 dpb. (8V) Serum IgG responses against group 1 at 14 dpc. (8W-8X) Serum IgA response against (8W) group 1 and (8X) group 2 HAs. Statistically significant differences between IGIP-H1att and caLen are depicted red asterisks.

FIGS. 9A-9C are plots showing IgA serum responses 20 dpb in IGIP-H1att- and H1caLen-vaccinated mice. Same set of samples from FIG. 8A-8D, probed for IgA antibodies against (9A) H1, (9B) H5 and (9C) H9 using the influenza antigen array. The reactivity of serum samples is expressed as described in FIGS. 8A-8D and results shown as MFI±SD. IGIP-H1att samples in blue dots/bars. H1caLen samples in grey dots/bars. PBS control samples in white dots/bars. No significant differences were observed.

FIG. 10A-10E are plots showing IgG serum responses 14 dpc in IGIP-H1att- and H1caLen-vaccinated mice. Mice (n=8/group) were bled at 14 dpc and the sera were collected and used to evaluate antibody titers, as described in FIG. 8A-8D IGIP-H1att samples in blue dots/bars. H1caLen samples in grey dots/bars. PBS control samples in white dots/bars. (10A) HAI, VN, and VN Nluc titers. Levels of IgG antibodies against (10B) H1, (10C) H5, (10D) H9 and (10E) group 2 HAs. The reactivity of serum samples in main graphs and insets is expressed as described in FIG. 8A-8D and results are shown as MFI±SD. Statistically significant differences between IGIP-H1att and caLen are marked with asterisks.

FIGS. 11A-11C are plots showing IgA serum responses 14 dpc in IGIP-H1att- and H1caLen-vaccinated mice. Same set of samples from 10A-10E probed for IgA antibodies against (11A) H1, (11B) H5 and (11C) H9. The reactivity of serum samples is expressed as described in FIG. 8A-8D and results shown as MFI±SD. The statistically significant differences between IGIP-H1att and caLen are depicted with asterisks.

FIG. 12A-12D are plots showing Mucosal IgG and IgA responses 14 dpc in IGIP-H1att- and H1caLen-vaccinated mice. Mice (n=4/group) were humanely euthanized at 14 dpc, and nasal washes (12A, 12C) and BALFs (12B, 12D) were collected to evaluate the levels of IgG (12A, 12B) and IgA (12C, 12D) antibodies against H1 HAs on the protein microarray. The reactivity of samples in main graphs and insets is expressed as described in FIGS. 8A-8D and results shown as MFI±SD. The statistically significant differences between IGIP-H1att and caLen are depicted with asterisks.

FIGS. 13A-13C are plots showing the serum antibody responses against NA and IAV internal proteins. The same set of samples described in FIG. 8A-8D (20 dpb) and FIG. 10A-10E (14 dpc) were probed for anti-NA (13A, 13C) and anti-IAV internal proteins (13B) antibody responses against antigens on the protein microarrays panel as indicated on the graphs. (13C) a Data on N1 NA A/Egypt/2321NAMRU3/2007 (H5N1) antigen was not used for analyses due to high background. Statistically significant differences between IGIP-H1att and caLen marked with asterisks.

FIGS. 14A-14D Mucosal antibody responses against NA and IAV internal proteins. The same set of samples from FIGS. 12A-12D were probed for anti-NA (14A) IgG and (14B) IgA antibodies as well as anti-IAV internal protein (14C) IgG and (14D) IgA antibodies. NW, nasal washes. Insets correspond to combined BALF and NW data in which each dot corresponds to the average reactivity of each sample against anti-IAV internal proteins in the array. (14B, 14C) a Data on N1 NA A/Egypt/2321NAMRU3/2007 (H5N1) antigen was not used for analyses due to high background. The statistically significant differences between IGIP-H1att and caLen are depicted with asterisks.

### DETAILED DESCRIPTION OF THE INVENTION

### I. Definitions

As used herein, “attenuated” refers to procedures that weaken an agent of disease (a pathogen). An attenuated virus is a weakened, less vigorous virus. A vaccine against a viral disease can be made from an attenuated, less virulent strain of the virus, a virus capable of stimulating an immune response and creating immunity but not causing illness or less severe illness. Attenuation can be achieved by chemical treatment of the pathogen, through radiation, or by genetic modification, using methods known to those skilled in the art. Attenuation may result in decreased proliferation, attachment to host cells, or decreased production or strength of toxins.

As used herein, the term “nucleic acid(s)” refers to any nucleic acid containing molecule, including, but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. In accordance with standard nomenclature, nucleic acid sequences are denominated by either a three letter, or single letter code as indicated as follows: adenine (Ade, A), thymine (Thy, T), guanine (Gua, G) cytosine (Cyt, C), uracil (Ura, U).

As used herein, the term “polynucleotide” refers to a chain of nucleotides of any length, regardless of modification (e.g., methylation).

As used herein, the term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid molecule encoding,” refers to the order or sequence of nucleotides along a strand of nucleotides. The order of these nucleotides can determine the order of amino acids along the polypeptide (protein) chain. The nucleotide sequence can thus code for the amino acid sequence.

As used herein, “heterologous” means derived from a different species.

As used herein, “homologous” means derived from the same species. For example, a homologous trait is any characteristic of organisms that is derived from a common ancestor. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

As used herein, “autologous” means derived from self.

As used herein, “endogenous” means a substance that originates from within an organism, tissue, or cell.

As used herein, “exogenous” means a substances that originates from outside an organism, tissue, or cell.

As used herein a “recombinant protein” is a protein derived from recombinant DNA.

As used herein “recombinant DNA” a refers to DNA molecules that is extracted from different sources and chemically joined together; for example DNA including a gene from one source may be recombined with DNA from another source. Recombinant DNA can be all heterologous DNA or a combination of homologous and heterologous DNA. The recombinant DNA can be integrated into and expressed from a cell's chromosome, or can be expressed for an extra-chromosomal array such as a plasmid.

As used herein, a “nucleic acid sequence alteration” can be, for example, a substitution, a deletion, or an insertion of one or more nucleotides. An “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, the term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, “identity,” as known in the art, is a relationship between two or more polynucleotide or polypeptide sequences, as determined by comparing the sequences. In the art, “identity” also means the degree of sequence relatedness between the polynucleotide or polypeptide as determined by the match between strings of such sequences. “Identity” can also mean the degree of sequence relatedness of a polynucleotide or polypeptide compared to the full-length of a reference polynucleotide or polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H, and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polynucleotides or polypeptides of the present disclosure.

By way of example, a polynucleotide or polypeptide sequence may be identical to the reference sequence, that is be 100% identical, or it may include up to a certain integer number of nucleotides or amino acid alterations as compared to the reference sequence such that the % identity is less than 100%. Such alterations are selected from: at least one deletion, substitution, including conservative and non-conservative substitution, or insertion, and wherein said alterations may occur at the 5′ or 3′ end of the polynucleotide, or amino- or carboxy-terminal positions of the reference polypeptide sequence or anywhere between those terminal positions, interspersed either individually among the nucleic acids or amino acids in the reference sequence or in one or more contiguous groups within the reference sequence. The number of nucleotide or amino acid alterations for a given % identity is determined by multiplying the total number of nucleic acids or amino acids in the reference polynucleotide or polypeptide by the numerical percent of the respective percent identity (divided by 100) and then subtracting that product from said total number of nucleic acids or amino acids in the reference polynucleotide or polypeptide.

As used herein, “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “treat” means to prevent, reduce, decrease, or ameliorate one or more symptoms, characteristics or comorbidities of an age-related disease, disorder or condition; to reverse the progression of one or more symptoms, characteristics or comorbidities of an age related disorder; to halt the progression of one or more symptoms, characteristics or comorbidities of an age-related disorder; to prevent the occurrence of one or more symptoms, characteristics or comorbidities of an age-related disorder; to inhibit the rate of development of one or more symptoms, characteristics or comorbidities or combinations thereof.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

As used herein, an “adjuvant” is a substance that increases the ability of an antigen to stimulate the immune system.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

A used herein, the term “pharmaceutically-acceptable carrier” means one or more compatible solid or liquid fillers, diluents or encapsulating substances which are suitable for administration to a human or other vertebrate animal.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### II. Compositions

Recombinant constructs, viral genomes including the recombinant constructs, recombinant viruses including the constructs, and vaccine formulations formed thereof are provided.

A. IGIP Expression Constructs

Expression constructs encoding IGIP are provided. The disclosed recombinant viruses typically have a nucleic sequence, typically a genomic nucleic acid sequence, encoding an IGIP polypeptide operably linked to an expression control sequence leading to transcription and translation of IGIP polypeptide by cells infected with the virus. Intermediate constructs, for example amplicons and forward and reverse genetic vectors including the constructs, and which can be used for engineering the viruses, are also provided.

The nucleic acid sequence encoding IGIP polypeptide is most typically linked to a sequence encoding a hemagglutinin or a neuraminidase, e.g., an HA or an NA as discussed in more detail below. The nucleic acid encoding the IGIP can be operable linked to the same or a different expression control sequence leading to the transcription and translation of the hemagglutinin or the neuraminidase. For example, in some embodiments, the construct encoding the IGIP and hemagglutinin or neuraminidase is polycistronic. The messenger RNA can be cleaved into individual messages after transcription, wherein the IGIP and hemagglutinin or neuraminidase are separately translated into proteins, or a single polypeptide chain (i.e., polyprotein) may be translated that is later cleaved to yield the individual proteins.

The nucleic acid sequence may further encode various additional elements including, but not limited to, signal peptides, polypeptide linkers, self-cleaving peptides, protease cleavage site sequences (e.g., viral, host cell, or otherwise supplied), reporters, etc.

1. IGIP Sequences

The disclosed constructs typically include a nucleic acid sequence encoding an IgA-inducing protein (IGIP) or a biologically functional fragment or variant thereof. IGIP was initially characterized in the bovine gastrointestinal associated lymphoid tissue (GALT) and has been shown to positively regulate IgA expression. See, e.g., U.S. Pat. Nos. 6,930,167 and 7,638,284. The disclosed recombinant viruses encode and express an IGIP polypeptide. Preferably, the IGIP polypeptide is secreted by infected host cells.

IGIP polypeptide sequences for a variety of species are known in the art. The endogenous proteins typically include a single peptide sequence at the N-terminal end. As discussed in more detail below, in some embodiments, the disclosed constructs include a nucleic acid sequence encoding an entire full-length IGIP protein including the naturally-occurring signal peptide sequence. In other embodiments, the disclosed constructs include a nucleic acid sequence encoding a biologically functional fragment or variant of a full-length IGIP protein. Portions of IGIP include contiguous residues of mature IGIP, e.g., 4, 7, 10, 15, 20 or more residues, but less than full length mature IGIP. Moreover, full length IGIP, or mature IGIP or portions thereof, may be linked, e.g., fused, to a heterologous peptide or polypeptide, for instance, to provide an IGIP fusion polypeptide. For example, mature IGIP may be linked to a peptide or non-peptide molecule that enhances the immune response to the fusion polypeptide.

In some embodiments, the disclosed constructs include a nucleic acid sequence encoding a mature IGIP or a biologically functional fragment thereof optionally, but preferably, fused to a heterologous signal peptide sequence (e.g., an alternative signal peptide that is not the signal peptide found associated with the IGIP in nature). In some embodiments, the heterologous signal peptide sequence enhances secretion of the IGIP polypeptide from host cells infected by the disclosed recombinant attenuated viruses.

Exemplary, non-limiting IGIP peptide sequences include

bovine IGIP, for example:
(SEQ ID NO: 7:, UniProtKB-POC712 (IGIP_BOVINE))
MKKRSVSGCNITILAVVFSHLSAGNSPCGNQANVLCISRLEFVQYQS,
and
mature forms thereof such as
(SEQ ID NO: 4)
GNSPCGNQANVLCISRLEFVQYQS,
and
(SEQ ID NO: 1)
NSPCGNQANVLCISRLEFVQYQS;
human IGIP, for example:
(SEQ ID NO: 8, UniProtKB-A6NJ69 (IGIP_HUMAN))
MCSYYHMKKRSVSGCNITIFAVMFSHLSAGKSPCGNQAN
VLCISRLEFVQYQS,
and
(SEQ ID NO: 9)
MKKRSVSGCNITIFAVMFSHLSAGKSPCGNQANVLCISRLEFVQYQS,
and
mature forms thereof such as
(SEQ ID NO: 5)
GKSPCGNQANVLCISRLEFVQYQS,
and
(SEQ ID NO: 2)
KSPCGNQANVLCISRLEFVQYQS;
and
mouse IGIP, for example:
(SEQ ID NO: 10, UniProtKB-A0A1B0GR74
(A0A1B0GR74_MOUSE))
MCSYYHMKKRSVLGCNITIFAVMFSHLSAGNSPCGNQATVLCISRLE
FVQYQS,
and
(SEQ ID NO: 11)
MKKRSVLGCNITIFAVMFSHLSAGNSPCGNQATVLCISRLEFVQYQS,
and
mature forms thereof such as
(SEQ ID NO: 6)
GNSPCGNQATVLCISRLEFVQYQS,
and
(SEQ ID NO: 3)
NSPCGNQATVLCISRLEFVQYQS;
and
Ferret IGIP and Swine IGIP, for example
(SEQ ID NO: 12)
MCSYYHMKKRSVSGCNITILAVVFSHLSAGNSPCGNQANVLCISRLE
FVQYQS,
and
mature forms thereof such as
(SEQ ID NO: 4)
GNSPCGNQANVLCISRLEFVQYQS,
and
(SEQ ID NO: 1)
NSPCGNQANVLCISRLEFVQYQS.

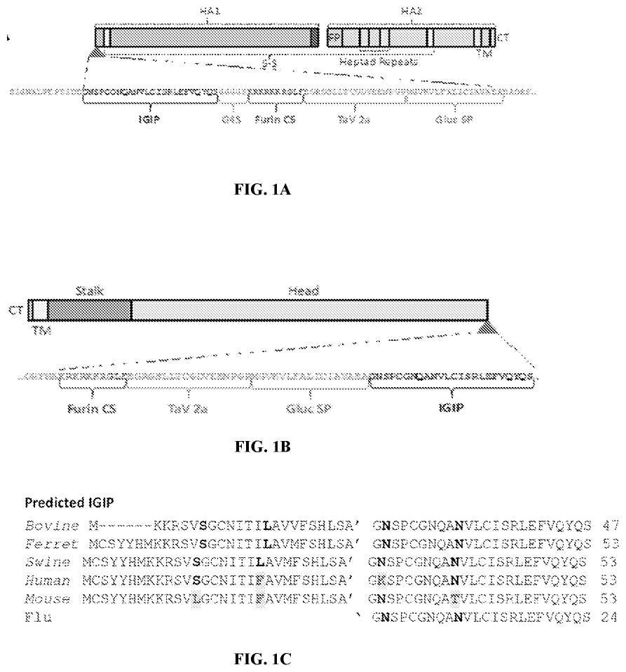

See, e.g., FIG. 1A.

Thus, in some embodiments, the nucleic acid construct encodes a polypeptide or fusion polypeptide thereof having the amino acid sequence of any one of SEQ ID NOS: 1-12, or a functional fragment thereof, or variant thereof with at least 70%, 75%, 80%, 85%, 90%, 95%, or more sequence identity to any one of SEQ ID NOS:1-12.

The disclosed compositions typically encode, and the cells infected with the disclosed recombinant viruses typically secrete an IGIP, a biologically active portion, an analog or a derivative thereof. A biologically active portion, an analog or a derivative of IGIP can have, for example, one or more substantially similar activities an IGIP polypeptide having any one of SEQ ID NOS:1-12. IGIP activities include, for example, the induction of IgA secretion from peripheral B cells stimulated with CD40L or CD40L-DAP3 cells and IL-2, CD40L and anti-IgM antibody, or CD40L, and/or enhances Ig production in vivo in animals, e.g., in guinea pigs, mice or a bovine-mouse chimera.

In particular embodiments, the IGIP polypeptide is SEQ ID NO:4, which is the IGIP polypeptide utilized the experiments described below. See, e.g., FIGS. 1B and 1C.

In some embodiments, the IGIP is a full length IGIP, or mature IGIP or portion thereof of another species. Such IGIP sequence are known in the art and available under the UniPro accession numbers provided in Table 1, below, each of which is specifically incorporated by reference herein in its entirety. Thus, in some embodiments, the nucleic acid construct encodes a polypeptide or fusion polypeptide thereof having the amino acid sequence of any one of the UniPro accession numbers provides in Table 1, or a functional fragment thereof, or variant thereof with at least 70%, 75%, 80%, 85%, 90%, 95%, or more sequence identity to any one of the UniPro accession numbers provides in Table 1.

TABLE 1
IGIP Accession Numbers by Organism

| Entry | Entry name | Organism |
|---|---|---|
| P0C7I2 | IGIP_BOVIN | Bos taurus (Bovine) |
| A6NJ69 | IGIP_HUMAN | Homo sapiens (Human) |
| A0A1S2ZD74 | A0A1S2ZD74_ERIEU | Erinaceus europaeus (Western |
| | | European hedgehog) |
| A0A1S3FIL9 | A0A1S3FIL9_DIPOR | Dipodomys ordii (Ord's kangaroo |
| | | rat) |
| M0R8G5 | M0R8G5_RAT | Rattus norvegicus (Rat) |
| H2R142 | H2R142_PANTR | Pan troglodytes (Chimpanzee) |
| M3XFF0 | M3XFF0_FELCA | Felis catus (Cat) (Felis silvestris |
| | | catus) |
| A0A1B0GR74 | A0A1B0GR74_MOUSE | Mus musculus (Mouse) |
| D2GUP8 | D2GUP8_AILME | Ailuropoda melanoleuca (Giant |
| | | panda) |
| I3MUP2 | I3MUP2_ICTTR | Ictidomys tridecemlineatus |
| | | (Thirteen-lined ground squirrel) |
| | | (Spermophilus tridecemlineatus) |
| H0WMM9 | H0WMM9_OTOGA | Otolemur garnettii (Small-eared |
| | | galago) (Garnett's greater |
| | | bushbaby) |
| G7P8G0 | G7P8G0_MACFA | Macaca fascicularis (Crab-eating |
| | | macaque) (Cynomolgus monkey) |
| A0A0D9S9H1 | A0A0D9S9H1_CHLSB | Chlorocebus sabaeus (Green |
| | | monkey) (Cercopithecus sabaeus) |
| A0A2I3FVY4 | A0A2I3FVY4_NOMLE | Nomascus leucogenys (Northern |
| | | white-cheeked gibbon) |
| | | (Hylobates leucogenys) |
| A0A2I3N620 | A0A2I3N620_PAPAN | Papio anubis (Olive baboon) |
| A0A2R8ZH58 | A0A2R8ZH58_PANPA | Pan paniscus (Pygmy |
| | | chimpanzee) (Bonobo) |
| A0A5F4D591 | A0A5F4D591_CANLF | Canis lupus familiaris (Dog) |
| | | (Canis familiaris) |
| A0A452DU75 | A0A452DU75_CAPHI | Capra hircus (Goat) |
| W5PVZ1 | W5PVZ1_SHEEP | Ovis aries (Sheep) |
| H2PGR5 | H2PGR5_PONAB | Pongo abelii (Sumatran |
| | | orangutan) (Pongo pygmaeus |
| | | abelii) |
| A0A5F9DK02 | A0A5F9DK02_RABIT | Oryctolagus cuniculus (Rabbit) |
| F6T2B8 | F6T2B8_MACMU | Macaca mulatta (Rhesus |
| | | macaque) |
| A0A2K6JMH2 | A0A2K6JMH2_RHIBE | Rhinopithecus bieti (Black snub- |
| | | nosed monkey) (Pygathrix bieti) |
| A0A2K6SBM8 | A0A2K6SBM8_SAIBB | Saimiri boliviensis boliviensis |
| | | (Bolivian squirrel monkey) |
| A0A2K6NRZ1 | A0A2K6NRZ1_RHIRO | Rhinopithecus roxellana (Golden |
| | | snub-nosed monkey) (Pygathrix |
| | | roxellana) |
| A0A2K6AXS1 | A0A2K6AXS1_MACNE | Macaca nemestrina (Pig-tailed |
| | | macaque) |
| A0A2K5JLQ6 | A0A2K5JLQ6_COLAP | Colobus angolensis palliatus |
| | | (Peters' Angolan colobus) |
| A0A2K5BZW7 | A0A2K5BZW7_AOTNA | Aotus nancymaae (Ma's night |
| | | monkey) |
| A0A2K5XC82 | A0A2K5XC82_MANLE | Mandrillus leucophaeus (Drill) |
| | | (Papio leucophaeus) |
| A0A2R8MQW5 | A0A2R8MQW5_CALJA | Callithrix jacchus (White-tufted- |
| | | ear marmoset) |
| A0A384C4H7 | A0A384C4H7_URSMA | Ursus maritimus (Polar bear) |
| | | (Thalarctos maritimus) |
| A0A2K5L2B0 | A0A2K5L2B0_CERAT | Cercocebus atys (Sooty |
| | | mangabey) (Cercocebus |
| | | torquatus atys) |
| A0A2K6GPH5 | A0A2K6GPH5_PROCO | Propithecus coquereli (Coquerel's |
| | | sifaka) (Propithecus verreauxi |
| | | coquereli) |
| A0A2K5PM08 | A0A2K5PM08_CEBCA | Cebus capucinus imitator |
| A0A1U7TKP3 | A0A1U7TKP3_TARSY | Tarsius syrichta (Philippine |
| | | tarsier) |
| A0A2U3V0T1 | A0A2U3V0T1_TURTR | Tursiops truncatus (Atlantic |
| | | bottle-nosed dolphin) (Delphinus |
| | | truncatus) |
| A0A2U3VRT9 | A0A2U3VRT9_ODORO | Odobenus rosmarus divergens |
| | | (Pacific walrus) |
| A0A340XR42 | A0A340XR42_LIPVE | Lipotes vexillifer (Yangtze river |
| | | dolphin) |
| A0A3Q7V077 | A0A3Q7V077_VULVU | Vulpes vulpes (Red fox) |
| A0A452R9X3 | A0A452R9X3_URSAM | Ursus americanus (American |
| | | black bear) (Euarctos |
| | | americanus) |
| A0A2Y9SEK8 | A0A2Y9SEK8_PHYMC | Physeter macrocephalus (Sperm |
| | | whale) (Physeter catodon) |
| A0A2U3XJ43 | A0A2U3XJ43_LEPWE | Leptony chotes weddellii |
| | | (Weddell seal) (Otaria weddellii) |
| A0A3Q7WFZ6 | A0A3Q7WFZ6_URSAR | Ursus arctos horribilis |
| A0A452C9N3 | A0A452C9N3_BALAS | Balaenoptera acutorostrata |
| | | scammoni (North Pacific minke |
| | | whale) (Balaenoptera davidsoni) |
| A0A2Y9PMY5 | A0A2Y9PMY5_DELLE | Delphinapterus leucas (Beluga |
| | | whale) |
| A0A3Q2GU56 | A0A3Q2GU56_HORSE | Equus caballus (Horse) |
| A0A671EQR0 | A0A671EQR0_RHIFE | Rhinolophus ferrumequinum |
| | | (Greater horseshoe bat) |
| A0A673T5I4 | A0A673T5I4_SURSU | Suricata suricatta (Meerkat) |
| A0A3Q7MM50 | A0A3Q7MM50_CALUR | Callorhinus ursinus (Northern fur |
| | | seal) |
| A0A341BZM5 | A0A341BZM5_NEOAA | Neophocaena asiaeorientalis |
| | | asiaeorientalis (Yangtze finless |
| | | porpoise) (Neophocaena |
| | | phocaenoides subsp. |
| | | asiaeorientalis) |
| A0A3Q0D6R0 | A0A3Q0D6R0_MESAU | Mesocricetus auratus (Golden |
| | | hamster) |
| A0A4W2HTV5 | A0A4W2HTV5_BOBOX | Bos indicus × Bos taurus (Hybrid |
| | | cattle) |
| A0A2Y9I0T8 | A0A2Y9I0T8_NEOSC | Neomonachus schauinslandi |
| | | (Hawaiian monk seal) (Monachus |
| | | schauinslandi) |
| A0A2Y9DU85 | A0A2Y9DU85_TRIMA | Trichechus manatus latirostris |
| | | (Florida manatee) |
| A0A158RFU4 | A0A158RFU4_HUMAN | Homo sapiens (Human) |
| A0A6D2WUB2 | A0A6D2WUB2_PONAB | Pongo abelii (Sumatran |
| | | orangutan) (Pongo pygmaeus |
| | | abelii) |
| A0A6D2WRA5 | A0A6D2WRA5_PANTR | Pan troglodytes (Chimpanzee) |
| A0A2Y9IL60 | A0A2Y9IL60_ENHLU | Enhydra lutris kenyoni |
| A0A2I2ZWB5 | A0A2I2ZWB5_GORGO | Gorilla gorilla gorilla (Western |
| | | lowland gorilla) |
| G7MUX7 | G7MUX7_MACMU | Macaca mulatta (Rhesus |
| | | macaque) |
| A0A2K5UH90 | A0A2K5UH90_MACFA | Macaca fascicularis (Crab-eating |
| | | macaque) (Cynomolgus monkey ) |
| A0A5J5N3V2 | A0A5J5N3V2_MUNRE | Muntiacus reevesi (Reeves' |
| | | muntjac) (Cervus reevesi) |
| A0A484GL75 | A0A484GL75_SOUCH | Sousa chinensis (Indo-pacific |
| | | humpbacked dolphin) (Steno |
| | | chinensis) |
| A0A5N3WCW9 | A0A5N3WCW9_MUNMU | Muntiacus muntjak (Barking |
| | | deer) (Indian muntjac) |
| L8IAG8 | L8IAG8_9CETA | Bos mutus (wild yak) |

2. Hemagglutinin and Neuraminidase

The disclosed constructs typically include a nucleic acid sequence encoding hemagglutinin or neuraminidase.

a. Hemagglutinin

Influenza hemagglutinin (also referred to herein as H and HA) is a homotrimeric glycoprotein found on the surface of influenza viruses and is integral to its infectivity. It is a Class I fusion protein, having multifunctional activity as both an attachment factor and membrane fusion protein. HA is responsible for binding influenza virus to sialic acid on the surface of target cells, such as cells in the upper respiratory tract or erythrocytes, causing as a result the internalization of the virus. HA is also responsible for the fusion of the viral envelope with the late endosomal membrane once exposed to low pH (5.0-5.5).

Hemagglutinin in influenza A has at least 18 different subtypes, H1 through H18. See also Russell, et al., Trends in Microbiol. 26(10):P841-853 doi:https://doi.org/10.1016/j.tim.2018.03.005 (2018), which is specifically incorporated by references herein in its entirety.

Despite the lack of subtypes, influenza B virus undergoes antigenic variation through genetic reassortment among cocirculating strains of different lineages and antigenic drift from cumulative mutations. The antigenic structures of influenza B virus HAs have been studied by sequence analysis of both naturally occurring variants and antibody-selected escape variants. See., e.g., Wang, et al., Journal of Virology, 82(6):3011-3020; DOI: 10.1128/JVI.02477-07 (2008).

The disclosed constructs can encode any of the different HA subtypes, e.g., any one of H1 through H18, or any influenza B HA. In some embodiments, the construct includes an influenza viral nucleic acid sequence encoding any one of H1 through H18 or an influenza B HA. In other embodiments, the construct includes an alternative sequence, e.g., a codon optimized nucleic acid sequence encoding any one of H1 through H18 or an influenza B HA.

H1, H2 and H3, and influenza B HA commonly found in humans, thus particularly for vaccine applications for improving human health, the construct may encode H1, H2, H3, or an influenza B HA. Constructs encoding H1, H3 or an influenza B HA and IGIP were tested in the experiments described in the working Examples below.

As discussed in more detail below, segment 6 of the influenza A virus encodes only the NA protein, while that of influenza B virus encodes both the NA protein and, in a −1 alternate reading frame, the NB matrix protein, which is an integral membrane protein corresponding to the influenza A virus M2 protein (Bouvier and Palese, Vaccine, 26(Suppl 4): D49-D53 (2008). Thus, in embodiments utilizing influenza B NA, the construct may further encode an NB protein.

b. Neuraminidase

Influenza neuraminidase (also referred to herein as N and NA) is found on the surface of influenza viruses and enables the virus to be released from the host cell. Neuraminidases are Glycoside hydrolase family 34 CAZY GH_34 enzymes that cleave sialic acid groups from glycoproteins and are required for influenza virus replication. Neuraminidase prevents self-aggregation by removing the carbohydrate from the viral envelope thus facilitating the mobility of the virus to and from the site of infection. There are at least 11 different neuraminidase subtypes, N1 through N11. See also McAuley, et al., Front Microbiol. 10: 39, doi: 10.3389/fmicb.2019.00039 (2019), which is specifically incorporated by references herein in its entirety.

Despite the lack of subtypes, numerous NA sequences have also been identified in influenza B strains, see, e.g., Correia, et al., Virology, 522:122-130, doi: 10.1016/j.virol.2018.07.002 (2018); Gillian, et al., Virology, 177(2):578-587,doi: 10.1016/0042-6822(90)90523-T (1990)).

The disclosed constructs can encode any of the different NA subtypes, e.g., any one of N1 through N11, or an influenza B NA. In some embodiments, the construct includes an influenza viral nucleic acid sequence encoding any one of N1 through N11, or an influenza B NA. In other embodiments, the construct includes an alternative sequence, e.g., a codon optimized nucleic acid sequence encoding any one of N1 through N11, or an influenza B NA.

N1, N2, and influenza B NA are commonly found in humans, thus particularly for vaccine applications for improving human health, the construct may encode N1, N2, or influenza B NA. Constructs encoding N1, N2, or an influenza B NA and IGIP were tested in the experiments described in the working Examples below.

3. Signal Peptides

In preferred embodiments, host cells infected by the recombinant virus secrete IGIP. Thus, constructs encoding the IGIP typically include a nucleic acid sequence encoding a signal peptide (also referred to as a signal sequence, targeting signal, localization signal, localization sequence, transit peptide, leader sequence or leader peptide) operably linked thereto. The hemagglutinin or neuraminidase protein also typically includes a signal peptide, thus, the nucleic acid sequences encoding the hemagglutinin or neuraminidase also typically include a nucleic acid sequence encoding a signal peptide operably linked thereto.

Signal peptides are short peptide sequences (usually 16-30 amino acids long), typically present at the N-terminal end of the protein. The core of the signal peptide typically contains a stretch of hydrophobic amino acids (about 5-16 residues long) that has a tendency to form a single alpha-helix. In addition, many signal peptides begin with a positively charged stretch of amino acids, which may help to enforce proper topology of the polypeptide during translocation. At the end of the signal peptide there can be a stretch of amino acids that is recognized and cleaved by signal peptidase during or after completion of translocation to generate a free signal peptide and a mature protein.

The signal sequence of the IGIP can be the signal sequence of the IGIP, or the naturally occurring IGIP signal sequence can be replaced with an alternative signal sequence. Most typically, the single sequence is one that facilitates secretion of IGIP by the infected host cell. Thus, in some embodiments, the IGIP signal peptide sequence is replaced by a signal peptide sequence that further improves its secretion from host cells. Similarly, the signal peptide sequence of the hemagglutinin or neuraminidase protein can be the signal sequence of the hemagglutinin or neuraminidase protein, or the naturally occurring hemagglutinin or neuraminidase protein signal peptide sequence can be replaced with an alternative signal peptide sequence.

Exemplary strategies include, but are not limited to, inserting a mature IGIP sequence in between the hemagglutinin or the neuraminidase signal sequence and its mature polypeptide sequence, and inserting a new heterologous signal sequence fused to the mature hemagglutinin or the neuraminidase protein sequence (see, e.g., FIG. 1B), or fusing the naturally occurring or a heterologous signal sequence to the mature IGIP sequence (see, e.g., FIG. 1C).

An exemplary heterologous signal sequence is the signal peptide of Gaussia luciferase (Gluc) (MGVKVLFALICIAVAEA (SEQ ID NO:26)), which is utilized the exemplary constructs of the experiments described below.

The fusion between the IGIP, HA, and NA and its respective signal peptide sequence may further include or more additional amino acid residues. For example, bioinformatics analysis can be used to predict improved cleavage with different signal peptide sequences, and may include, for example, the incorporation of one, two, three, four, five or more additional amino acids added/inserted at the C-terminus of the signal peptide sequence before the start of the IGIP, HA, and/or NA sequence. Likewise, in some embodiments, one, two, three, four, five or more additional amino acids are deleted/removed at the C-terminus of the signal peptide sequence before the start of the IGIP, HA, and/or NA sequence.

4. Protease Cleavage Sites

The nucleic acid constructs can encode one or more protein cleave sites. Exemplary protease cleavage sites include, but are not limited to, Furin cleavage sites. Furin cleavage sites have an RX(K/R)R (SEQ ID NO:13) consensus motif. As used herein, “X” or “x” in an amino acid sequence typically means any amino acid. See also, Zimmer, et al., J Virol., 76(18): 9218-9224 doi: 10.1128/JVI.76.18.9218-9224.2002 (2002), which is specifically incorporated by reference herein in its entirety.

The constructs exemplified in the experiments described below utilize a Furin cleavage site including the sequence KRKRKKR (SEQ ID NO:14).

Another exemplary protease cleavage site is a caspase-1 cleavage site, which may have a consensus motif of X-Glu-X-Asp (X-E-X-D) (SEQ ID NO:30). See, e.g., Shen, et al., Atherosclerosis, 210(2):422-429 (2010). doi:10.1016/j.atherosclerosis.2009.12.017.

5. Self-Cleaving Peptides

The nucleic acid constructs can encode one or more self-cleavage peptides. Exemplary self-cleaving peptides include, but are not limited to, 2A self-cleaving peptides. 2A self-cleaving peptides have a consensus motif DxExNPGP (SEQ ID NO:15). The constructs exemplified in the experiments described below utilize a Thosea asigna virus 2A peptide sequence including the sequence EGRGSLLTCGDVEENPGP (SEQ ID NO:16). Other 2A peptide sequences include P2A (ATNFSLLKQAGDVEENPGP (SEQ ID NO:17)), E2A (QCTNYALLKLAGDVESNPGP (SEQ ID NO:18)), and F2A (VKQTLNFDLLKLAGDVESNPGP (SEQ ID NO:19)). The cleavage is triggered by breaking of the peptide bond between the Proline (P) and Glycine (G) in C-terminal of 2A peptide, resulting in the peptide located upstream of the 2A peptide having extra amino acids on its C-terminal end while the peptide located downstream the 2A peptide has an extra Proline on its N-terminal end. Adding the optional linker, e.g., Gly-Ser-Gly on the N-terminal of a 2A peptide can help with efficiency.

6. Peptide Linkers

The nucleic acid constructs can encode one or more peptide linkers to e.g., separate various elements encoded by the construct.

Exemplary flexible linkers include, but are not limited to, Gly-Ser, Gly-Ser-Gly, Ala-Ser, Gly-Leu-Phe, Gly-Ser-Gly-Ser (SEQ ID NO:20), Gly-Gly-Gly-Ser (SEQ ID NO:21), Gly-Gly-Gly-Gly-Ser (SEQ ID NO:22), (Gly₄-Ser)₂ (SEQ ID NO:23), (Gly₄-Ser)₄ (SEQ ID NO:24), (Gly-Gly-Gly-Gly-Ser)₃ (SEQ ID NO:25). The constructs exemplified below utilize a Gly-Gly-Gly-Gly-Ser (SEQ ID NO:22) and Gly-Leu-Phe linkers.

7. Reporters

In some embodiments, the constructs further encoding a reporter gene. Reporter genes are typically genes that are not present or expressed in the host cell. The reporter gene typically encodes a protein which provides for some phenotypic change or enzymatic property. Examples of such genes are provided in K. Weising et al. Ann. Rev. Genetics, 22, 421 (1988), and include, but are not limited to, carcinoembryonic antigen, secreted alkaline phosphatase, and the beta subunit of chorionic gonadotropin, glucuronidase (GUS), luciferase (e.g., Gaussia Luciferase (GLuc), Nanoluciferase (NLuc), and fluorescent proteins such as green fluorescent protein (GFP), enhanced green fluorescent protein (eGFP), turbo red fluorescent protein (TurboRFP), etc.

The reporter can serve as a measure or monitor of in vivo viral activity. For example, these reporters are released by infected cells into the blood, and can be measured peripherally to determine viral activity (Phuong, et al., Cancer Res., 63:2462-2469 (2003); Peng, et al., Nat. Med., 8:527-531 (2002); Shashkova, et al., Cancer Gene Ther., 15:61-72 (2008); Hiramatsu, et al., Cancer Science, 100, 1389-1396 (2005)).

It will also be appreciated that in some embodiments, the reporter is encoded by the virus's genome, but is not part of the IGIP-encoding construct. For example, in some embodiments, the IGIP encoding sequence is co-located with HA and reported encoding sequence is co-located with the sequence encoding NA on a different viral genomic segment; or the IGIP encoding sequence is co-located with NA and reported encoding sequence is co-located with the sequence encoding HA on a different viral genomic segment.

Additionally or alternatively, the reporter encoding sequence can be on a different viral genomic segment, and thus not co-located with IGIP, NA, or HA. For example, in some embodiments, the sequence encoding the reporter is located upstream or downstream of another influenza gene, for example, the PB1 gene (segment 2) or NS1 gene (segment 8). See, e.g., U.S. Published Application No. 2014/0161771.

8. Additional Elements and Exemplary Viral Genomic Segments

Suitable virus backbones are discussed in more detail below. Most typically, the recombinant viruses include genomic segment of an influenza virus engineered to encode, express, and preferably secret mature IGIP polypeptide. Thus, the disclosed nucleic acid constructs, including, but not limited to recombinant influenza genomic segments, are can include (1) the expression control feature(s) necessary and sufficient for, and (2) nucleic acid sequence arranged in an orientation suitable for, IGIP expression in host cells infected by the recombinant virus. The constructs may also include the features necessary and sufficient for replication of the construct and packaging thereof into a virion.

Most typically, the IGIP expression construct is engineered for expression along with hemagglutinin, e.g., on genomic segment 4, or neuraminidase, e.g., segment 6 of the influenza genome. The segments are designed so that the hemagglutinin or neuraminidase are also expressed by the recombinant virus.

Influenza viruses are negative-sense, single-stranded RNA (-ssRNA) viruses. They have genomes made of RNA, which are single instead of double-stranded. Their genomes are negative sense, meaning that messenger RNA (mRNA) is synthesized from the genome by the viral enzyme RNA-dependent RNA polymerase (RdRp), also called RNA replicase, which is encoded by all negative (−) ssRNA viruses. Thus, as used herein, a single stranded nucleic acid “encodes” the described features (e.g., IGIP, H, N, linkers, cleavage sites, etc.) where the strand is either the positive sense (e.g., coding strand) or the negative sense (e.g., template strand).

For ease of description, the following exemplary segments are presented in the 5′ to 3′ orientation to reflect the positive sense nature of the encoded protein sequences in influenza genomic segments as well as in nucleic acids as they are also incorporated into double stranded amplicons, cloning, and However, it will be appreciated that the reverse orientation as well as the complementary strand are also provided, and nucleic acids may also be described in the 3′-5′orientation when specified.

Exemplary orientations of the nucleic acid constructs and viral genomic segments include, for example,
‘5 nucleic acid sequence encoding IGIP polypeptide-nucleic acid sequence encoding H or N 3’; and
‘5 nucleic acid sequence encoding H or N-nucleic acid sequence encoding IGIP polypeptide 3’;

In some embodiments, a 5′ untranslated region (UTR), 3′ untranslated region (UTR), or a combination thereof, e.g., from influenza virus are included at the 5′ and 3′ ends respectively of the nucleic acid.

In particular embodiments, the nucleic acid encodes H and includes the 5′ UTR and 3′ UTR of segment 4 of an influenza virus at the 5′ and 3′ ends respectively of the nucleic acid.

In other embodiments, the nucleic acid encodes N and includes the 5′ UTR and 3′ UTR of segment 6 of an influenza virus at the 5′ and 3′ ends respectively of the nucleic acid.

Exemplary plasmid constructs presented in the 5′-3′ orientation used for generation of recombinant genomic segments capable of IGIP and hemagglutinin or neuraminidase expression in infected host cells are exemplified in the working examples below, and feature the following sequences in the following orientation:
5′-UTR of segment 4, signal peptide of HA, IGIP mature peptide sequence, linker sequence (e.g., G4S), cleavage site (e.g., furin-recognition CS), self-cleaving peptide sequence (e.g., the Thosea assigna virus 2A protein sequence), signal peptide sequence (e.g., single peptide of Gaussia luciferase (Gluc)), HA open reading frame, a cloning spacer, and the 3′-UTR of segment 4 (FIG. 1B), and
5′-UTR of segment 6 followed by a cloning spacer downstream the 5′ UTR, NA open reading frame (and optionally NB, where the NA is an influenza B NA), linker sequence (e.g., G4S), cleavage site (e.g., furin-recognition CS), self-cleaving peptide sequence (e.g., the Thosea assigna virus 2A protein sequence), signal peptide sequence (e.g., single peptide of Gaussia luciferase (Gluc)), IGIP mature peptide sequence, and the 3′-UTR of segment 6 (FIG. 1C).

The orientation is not believed to be vital, and thus alternative orientations are also provided. However, the orientations as described in the figures may be the most suitable for the disclosed vaccine compositions and methods.

In particular embodiments, the processed IGIP protein includes an additional 12 aa C-terminal tail (G4S(K/R)7), yielding the sequence

SEQ ID NO: 31)
GNSPCGNQANVLCISRLEFVQYQSGGGGSKRKRKKR.

B. Isolated Nucleic Acids, Vectors, and Amplicons

The disclosed IGIP constructs can be isolated nucleic acids, vectors, amplicons, viral genomes, etc., and many exist in multiple forms during the manufacture and use of the disclosed recombinant viruses. For example, the constructs can be linear or circular, single stranded or double stranded nucleic acid molecules formed of RNA or DNA, in the sense or antisense sequence orientations (e.g., positive or negative strand).

As used herein, “isolated nucleic acid” refers to a nucleic acid that is separated from other nucleic acid molecules that are present in a genome, including nucleic acids that normally flank one or both sides of the nucleic acid in a genome. An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus, or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acids can be in sense or antisense orientation, or a combination thereof. Thus, the nucleic acids can be the coding sequence, complementary thereto, or a combination thereof. The nucleic acid sequences can be discussed relative to a reference sequence encoding an IGIP polypeptide or variant thereof, optionally further including additional elements including, but not limited to, 5′ and/or 3′ UTRs, those encoding signal peptide(s), protease cleavage site(s), self-cleaving peptide(s), peptide linker(s), reports, etc. as discussed herein. Reference sequences include, for example, the nucleotide sequence of IGIP and other elements of the construct include those which are known in the art and discussed above.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid.

Nucleic acids can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, HA, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of several techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection.

C. Recombinant Viruses

The disclosed recombinant viruses capable of expressing IGIP in infected host cells are typically formed from an influenza virus backbone, however, it will be appreciated that IGIP could be produced from other live recombinant vectors used for vaccines e.g., vaccinia, adeno, herpes, paramyxovirus 5, Newcastle disease virus, measles, etc. Thus, although the following, description is particularly detailed with respect to influenza, the same or similar strategies can be utilized in an alternative recombinant viral backbone such as vaccinia, adeno, herpes, paramyxovirus 5, Newcastle disease virus, measles, yellow fever, mumps, rubella, rotavirus, etc. and suitable expression constructs and live recombinant viruses thereof for expressing IGIP, and their use in vaccines are also provided. See, e.g., Nascimento and Liete, Braz J Med Biol Res., 45(12): 1102-1111 (2012), Choi and Chang, et al., Clin Exp Vaccine Res. 2(2): 97-105 (2013), Souza, et al., Braz J Med Biol Res 38(4):509-022 (2005), each of which is specifically incorporated by reference in its entirety.

The disclosed recombinant viruses are typically discussed as live viruses, however, inactivated viruses and viral components thereof are also provided.

Any naturally occurring or engineered influenza virus, preferably a type A or type B virus, can serve the backbone for the disclosed recombinant viruses. Most typically the virus is an attenuated virus. In some embodiments, the virus backbone is a naturally-occurring or other wildtype virus, and the addition of IGIP-expressing construct leads to attention of the virus. In preferred embodiments, the backbone virus is a virus that has already been attenuated. Addition of the IGIP-expressing construct into such live attenuated virus (LAIV) genomes may further attenuate the virus, or addition of the IGIP-expressing construct may not further attenuate the virus. Thus, in some embodiments, the level of attenuation of the recombinant IGIP-expressing virus is the same as the virus backbone with the IGIP-expressing virus, while in some embodiments, attenuation is increased.

1. Structure of the Recombinant Viruses

There are four types of influenza viruses: A, B, C and D. Human influenza A and B viruses cause seasonal epidemics of disease (known as the flu season) almost every winter in the United States (CDC website, “Types of Influenza Viruses”). Influenza A viruses are the only influenza viruses known to cause flu pandemics, i.e., global epidemics of flu disease. A pandemic can occur when a new and very different influenza A virus emerges that both infects people and has the ability to spread efficiently between people. Influenza type C infections generally cause mild illness and are not thought to cause human flu epidemics, and influenza D viruses primarily affect cattle and are not known to infect or cause illness in people.

The disclosed viruses are typically recombinant influenza A or influenza B viruses. Influenza A viruses are divided into subtypes based on two proteins on the surface of the virus: hemagglutinin (H or HA) and neuraminidase (N or NA). Influenza B viruses are not divided into subtypes, but instead are further classified into two lineages: B/Yamagata and B/Victoria. Similar to influenza A viruses, influenza B viruses can then be further classified into specific clades and sub-clades. Influenza B viruses generally change more slowly in terms of their genetic and antigenic properties than influenza A viruses, especially influenza A(H3N2) viruses. Influenza surveillance data from recent years shows co-circulation of influenza B viruses from both lineages in the United States and around the world. However, the proportion of influenza B viruses from each lineage that circulate can vary by geographic location.

Belonging to the family Orthomyxoviridae, the genomes of Type A and Type B include eight segments of single-stranded RNA of negative polarity (Webby R J, et al. (2007) Curr. Top. Microbiol. Immunol. 315: 67-83; Yamanaka K, et al. (1991) Proc Natl Acad Sci USA 88:5369-5373; Lopez-Turiso J A, et al. (1990) Virus Res 16: 325-337). The viruses have an envelope with a host-derived lipid bilayer and covered with about 500 projecting glycoprotein spikes with hemagglutinating and neuraminidase activities. These activities correspond to the two major surface viral glycoproteins: the hemagglutinin (HA) and neuraminidase (NA) (or NA+NB), present as homotrimers and homotetramers, respectively. Segments 4 and 6 encode for the HA and NA (or NA+NB) genes, respectively.

Within the envelope, a matrix protein (M1) and a nucleocapsid (NP) protein protect the viral RNA. The type designation (A, B, or C) is based upon the antigenic features of the M1 and NP proteins. Approximately half of the total genome encodes for the three viral polymerase proteins (segments 1, 2 and 3). Segment 5 encodes the NP protein. The three-polymerase subunits (PB1, PB2, and PA), the NP and the vRNA are associated in virions and infected cells in the form of viral ribonucleoprotein particles (vRNPs).

The two smallest segments (7 and 8) encode two genes each with overlapping reading frames, which are generated by splicing of the co-linear mRNA molecules. In addition to M1, segment 7 encodes for the proton pump transmembrane protein (M2), which has ion channel activity and is embedded in the viral envelope. Segment 8 encodes for NS1, a nonstructural protein that blocks the host's antiviral response, and the nuclear export protein (NS2 or NEP) a structural component of the viral particle. NEP/NS2 interacts with the cellular export machinery and participates in the assembly of virus particles. Recently, NEP/NS2 has also been implicated in playing a role in the regulation of influenza virus transcription and replication.

Thus, the eight RNA segments typically encode for 10-12 viral proteins, including two surface glycoproteins, HA and NA, M2, M1, NS2/NEP, NS1 and, in some influenza viruses (from an alternative translation start site in segment 1) the PB1-F2, an apoptosis modulatory protein (Arias C F, et al. (2009) Arch Med Res 40: 643-654; Zell R, (2006) Emerg Infect Dis 12: 1607-1608; author reply 1608-1609; Chen W, et al. (2001) Nat Med 7:1306-1312). Additional viral protein products include PB1-N40, derived from an alternative start site within the PB1 ORF, resulting in a protein product that lacks the first 39 aa of PB1, and PA-X, derived from the PA mRNA and consists of the N-terminal 191 aa of PA fused to 61 aa that result from +1 frameshifting (Jagger B W, et al. (2012) Science 337: 199-204; Yewdell J W, Ince W L (2012) Science 337: 164-165).

Thus, in some embodiments, the recombinant virus has a Type A background and encodes one or more, preferably, all of HA, NA, M2, M1, NS2/NEP, NS1, NP, PB1, PB2, PA, and IGIP proteins.

In some embodiments, the genome includes one or more, preferably all of:
Segment 1 encoding PB2;
Segment 2 encoding PB1 and optionally PB1-F2;
Segment 3 encoding PA and optionally PA-X;
Segment 4 encoding HA;
Segment 5 encoding NP;
Segment 6 encoding NA;
Segment 7 encoding M1 and M2; and
Segment 8 encoding NS1 and NEP;
   wherein either Segment 4 or Segment 6 is further engineered to express an IGIP.

In some embodiments, the recombinant virus has a Type B background and encodes one or more, preferably, all of HA, NA, NB, BM2, M1, NS2/NEP, NS1, NP, PB1, PB2, PA, and IGIP proteins.

In some embodiments, the genome includes one or more, preferably all of:
Segment 1 encoding PB1;
Segment 2 encoding PB2;
Segment 3 encoding PA;
Segment 4 encoding HA;
Segment 5 encoding NP;
Segment 6 encoding NA and NB;
Segment 7 encoding M1 and BM2; and
Segment 8 encoding NS1 and NEP;
   wherein either Segment 4 or Segment 6 is further engineered to express an IGIP.

a. Attenuated Virus Backbones

The disclosed recombinant viruses are typically live attenuated viruses (LAIV) suitable for use a vaccination protocol. In preferred embodiments, the backbone of the recombinant virus is one that is already attenuated relative to wildtype virus.

LAIVs for human use were originally independently obtained by the U.S. and Russia in the 1960's after serial passage in eggs resulting in viruses with cold-adapted, temperature-sensitive mutations that prevented their growth at temperatures higher than 35° C. and thus restricting virus replication to the nasal cavity, and subsequently have been obtained using a variety methods of techniques. The disclosed virus are typically attenuated virus are thus weaken and can exhibit, for example, reduced replication in the host.

The attenuated (att) virus background can be, for example, an available attenuated virus, or be a chimeric attenuated virus incorporating one or more attenuation features of one or more different attenuated viruses. Available LAIVs and attenuation features thereof include, for example, reassortant viruses e.g., carrying cold-adapted, temperature sensitive (ts) mutations of the master donor viruses A/Ann Arbor/6/60 for IAV and B/Ann Arbor/1/66 for IBV, NS1 truncation, elastase-dependent virus, and rearranged genomes. See, e.g., Finch, et al., Curr Top Microbiol Immunol. 386:205-35. doi: 10.1007/82_2014_404 (2015) and Rajao and Perez, Front Microbiol., 9: 123 doi: 10.3389/fmicb.2018.00123 (2018), and references discussed therein including, but not limited to, Maassab et al., Adv. Biotechnol. Process. 14, 203-242 (1990); Carter and Curran, Drugs 71, 1591-1622 (2011); Song et al., J Virol. 81, 9238-9248 (2007); Pena et al., J Virol. 85, 456-469 (2011); Loving et al., J Virol. 87, 9895-9903 (2013); Alam et al., J Virol. 88, 314-324 (2014); Gauger et al., Virology 471-473, 93-104 (2014); Santos et al., J Virol. 91, e00056-00017 (2017); Santos et al., Vaccine 35, 5637-5643 (2017); Solorzano et al., J Virol. 79, 7535-7543 (2005); Hai et al., J Virol. 82, 10580-10590 (2008); Richt and Garcia-Sastre, Curr. Top. Microbiol. Immunol. 333, 177-195 (2009); Kappes et al., Vaccine 30, 280-288 (2012); Pica et al., J. Virol. 86, 10293-10301 (2012); Shi et al., Vaccine 34, 350-357 (2016); Masic et al., Vaccine 28, 7098-7108 (2010); Pena et al., J. Virol. 87, 5118-5127 (2013); Nogales et al., J. Virol., 90, 6291-6302 (2016); Harding et al., MBio 8:e00669-17 (2017), as well as U.S. Published Application Nos. 2020/0023054, 2016/0030547, 2016/0022807, 2014/0302158, 2014/0161771, and 2011/0150912, and U.S. Pat. Nos. 10,434,166, 10,080,794, and 8,475,807, each of which is specifically incorporated by reference herein in its entirety.

An intranasal cold-adapted LAIV produced by MedImmune (FluMist) was first licensed in the U.S. in 2003, and quadrivalent version of FluMist was approved in 2012, though there is some evidence of reduced effectiveness in recent years which may be related to reduced stability and/or infectivity of the H1N1pdm09 strain (O'Donnell et al., J Virol. 88, 12374-12384 (2014)) or interference from other components in the vaccine. The “Leningrad” LAIV available in Russia since 1987 for children over 3 years of age, adults and the elderly continues to show superior effective protection in children compared to inactivated vaccines (Ghendon et al., Infect. Immun. 44, 730-733 (1984); Rudenko et al., Vaccine 34, 5436-5441 (2016) each of which is specifically incorporated by reference herein in its entirety).

A modified live vaccine against equine influenza virus (EIV) is a live-attenuated vaccine approved for use in animals. The vaccine has a cold adapted and temperature sensitive virus derived from the wild-type A/eq/Kentucky/1/91 (H3N8) EIV strain and has been proven safe and efficacious for use in horses in the U.S. (reviewed in Paillot, Vacc. (Basel) 2, 797-831 (2014) which is specifically incorporated by reference herein in its entirety).

In the background of IAVs, either avian- or swine-origin, incorporation of the single PB2 mutation (N265S) and the three PB1 mutations (K391E, D581G and A661T) from the MDV-A strain (most of these viruses naturally carry the NP D34G mutation) results in viruses with a ts phenotype in vitro, and adequate att phenotype in vivo was achieved after the incorporation of a C-terminal epitope tag in PB1 consisting of a 9-amino acid sequence derived from the H3 HA gene segment (HA tag) in the context of the ts mutations. Further studies showed that the IAV att viruses modified as described above (ts mutations plus HA-tag) are safe and efficacious as LAIVs in mice, chickens, and swine; and amenable for intranasal administration. In chickens, the IAV att vaccines were also suitable for immunization in ovo. Thus, in some embodiments, the att virus background includes one or more of PB2 mutation (e.g., N265 mutations, such as N265S) and/or PB1 mutation (e.g., K391 mutations, such as K391E; E581 mutations, such as E581G, and/or A661 mutations, such as A661T). See, also U.S. Published Application No. 2011/0150912.

In some embodiments, the att virus background includes a mutated influenza virus RNA-dependent RNA polymerase. A recombinant influenza virus can include, for example, a mutated PA polymerase subunit. In some aspects, a mutated PA subunit can have a mutation at position 59. For example, the mutation at position 59 can be an E to V mutation. Any of the mutations described herein can be present in the PA subunit.

In some embodiments, the att virus background includes a rearranged genome, for example, having one or more rearranged influenza genome segments. A rearranged genome segment may include rearrangement of any one or more of influenza genome segments 1, 2, 3, 4, 5, 6, 7 or 8.

Exemplary rearranged influenza genomes include, for example, those having a NS2 nucleic acid sequence, but wherein the NS2 nucleic acid sequence is removed from RNA segment 8 of the genome. In some embodiments, the NS2 nucleic acid sequence can be operably linked to the PB1 gene found on its normal/wild-type genomic segment. In some aspects, a rearranged genome includes an exogenous sequence operably linked to an influenza gene sequence, for example, a NS1 sequence. The NS1 sequence can be a truncated sequence. An exogenous sequence can be located downstream of a truncated NS1 sequence. An exogenous sequence may include any nucleic acid sequence. For example, the exogenous sequence can be a nucleic acid sequence from a different influenza strain. In some embodiments, the exogenous sequence can be a H5N1 hemagglutinin (HA) sequence or a neuraminidase sequence. In some embodiments, the exogenous sequence is a nucleic acid sequence from a virus other than influenza.

In some embodiments, recombinant att influenza viruses containing a rearranged genome have both a PB1-NS2 rearrangement and a NS1-exogenous sequence rearrangement.

Rearranged genome may include a cleavage site present within one or more rearranged genome segments. For example, a cleavage site can be present on the rearranged genome segment that contains PB1 and NS2 wherein the cleavage site is located between the PB1 and NS2 nucleic acid sequences. A rearranged genome segment may include a cleavage site between the exogenous sequence and the NS1 nucleic acid sequence. In some aspects, the cleavage site can be a CHYSEL (SEQ ID NO:27) site. CHYSEL (SEQ ID NO:27) sites include but are not limited to a foot-and-mouth disease virus 2A autoproteolytic (FMDV 2A) site.

Recombinant influenza viruses with a rearranged genome can be attenuated viruses. Attenuation of the influenza virus may be achieved through fusion of a cleavage site, through introduction of temperature-sensitive (ts) mutations in the PB2 (e.g., N265S) and PB1 (e.g., K391E, E581G, and A661T) genes, through introduction of mutations in PB1 that interferes with PB1-PA interaction (such as T6D), or through truncations in NS1. See also U.S. Published Application No. 2014/0161771.

Analogous mutations to those found in an IAV att alternative live virus vaccine have also be introduced into a prototypic B/Vic lineage strain, B/Brisbane/60/2008 (B/Bris). Specifically, mutations were engineered in PB2 K267S and PB1 (K391E, E580G and S660A), and in addition, the PB1 segment was modified with the C-terminal HA tag in the presence or absence of temperature sensitive mutations. Studies showed that the mutations E580G, and S660A and the C-terminal HA tag were stably maintained over 15 passages in SPF eggs and 20 passages in tissue culture cells, was stable, attenuated in vivo, and immunogenic. Thus, in some embodiment the att influenza B background is an Influenza B virus (including, but not limited to any Victoria lineage (for example, Influenza B/Brisbane, Influenza B/Malaysia) and/or Yamagata lineage (for example, Influenza B/Florida, Influenza B/Phuket, Influenza B/Shanghai, Influenza B/Massachusetts, or Influenza B/Wisconsin) virus disclosed herein) having substitution the glutamate at residue 580 and/or the serine at residue 660 of the PB1 segment of the viral polymerase with a nonpolar amino acid. For example, the substitution can be for a glycine, alanine, valine, leucine, or isoleucine. In particular embodiments, the substitution of the PB1 segment of the viral polymerase comprises an E580G and/or a S660A substitution. See also U.S. Published Application No. 2020/0023054.

In particular embodiments, the virus backbone is an OH/04 att backbone (Pena, et al., J Virol, 85(1):456-69 (2011), Loving, et al., Vaccine, 30(40):5830-8 (2012), Gauger, Virology, 471-473:93-104 (2014)), the cold-adapted Leningrad backbone (ca/LEN) (Ghendon, Infect Immun, 44(3):730-3 (1984), Isakova-Sivak, et al., Vaccines (Basel), 7(3) (2019)), or the backbone of B/Bris att (Wan, et al., J Virol, 92(21) (2018), Santos, J Virol, 91(12) (2017)), which were utilized in the experiments described below.

b. Hemagglutinin (H) and Neuraminidase (N) Subtypes

There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2). Currently circulating influenza A(H1N1) viruses are related to the pandemic 2009 HIN1 virus that emerged in the spring of 2009 and caused a flu pandemic (CDC 2009 HIN1 Flu website). This virus, scientifically called the “A(H1N1)pdm09 virus,” and more generally called “2009 H1N1,” has continued to circulate seasonally since then. These HIN1 viruses have undergone relatively small genetic changes and changes to their antigenic properties (i.e., the properties of the virus that affect immunity) over time.

The disclosed attenuated live virus can encode any of the 131 subtype combinations of H and N proteins, or H and N proteins for an influenza B. Thus, even the known attenuated live viruses can be further modified to exchange the hemagglutinin subtype and sequence, the neuraminidase subtype and sequence, or a combination thereof, preferably while maintaining the attenuation features that weaken the virus in the host. For example, in some embodiments, the genome of the recombinant virus is formed from segments 1-3, 5, 7-8 of a known attenuated virus, and one or both of segments 4 and 6 are replaced with a different segment 4 and/or segment 6 encoding a different hemagglutinin subtype and/or sequence, a different neuraminidase subtype and or sequence, or a combination thereof, wherein at segment 4 and/or segment 6 is also modified to encode and express IGIP.

H1, H2 and H3, and N1 and N2 are commonly found in humans, thus particularly for vaccine applications for improving human health, the recombinant live attenuated virus may feature H1, H2 or H3, in combination with N1 or N2. Subtypes confirmed in humans include H1N1, which caused Spanish flu in 1918, and Swine Flu in 2009; H2N2, which caused Asian Flu in 1957; H3N2, which caused Hong Kong Flu in 1968; H5N1, which caused Bird Flu in 2004; H7N7; H1N2, endemic in humans, pigs and birds; H9N2; H7N2; H7N3; H10N7; H7N9, rated in 2018 as having the greatest pandemic potential among the Type A subtypes; and H6N1.

Thus, in some embodiments, the recombinant virus is of the subtype H1N1, H2N2, H5N1, H7N7, H1N2, H9N2, H7N2, H7N3, H10N7, or H7N9, in a live virus background, preferably an attenuated live virus background, and engineered to express IGIP, preferably from segment 4 or segment 6 of the viral genome.

In some embodiments, the recombinant virus is an influenza B virus having an influenza B HA and an influenza B NA, in a live virus background, preferably an attenuated live virus background, and engineered to express IGIP, preferably from segment 4 or segment 6 of the viral genome.

2. Methods of Making Recombinant Viruses

Methods of making recombinant influenza viruses are known in the art, and can be employed in making the disclosed recombinant attenuated influenza viruses. See, for example, plasmid-based reverse genetics approaches, including, but not limited to, cloning of cDNA copies of viral RNA segments derived from influenza virus into reverse genetics plasmid vectors and amplicons, and the experimental procedures for the successful generation of recombinant influenza viruses are described in, e.g., Perez, et al., “Plasmid-Based Reverse Genetics of Influenza A Virus,” Methods of in Molecular Biology, Reverse Generics of RNA Viruses, Spring Protocols, doi: 10.1007/978-1-4939-6964-7_16 (2017); and Perez, et al., “Plasmid-Based Reverse Genetics of Influenza A Virus,” Methods of in Molecular Biology, Animal Influenza Virus, Spring Protocols, doi: 10.1007/978-1-0716-0346-8_4 (2020), U.S. Published Application No. 2014/0161771, U.S. Published Application No. 2016/0022807.

It is known that viruses, such as influenza, adenovirus, adeno-associated and lentivirus, can be made using multiple plasmids each containing different viral genes required to make the virus. Thus, for example, when a new strain of influenza evolves, the genes particular for that new strain, for example the HA or NA genes, can be cloned into appropriate plasmid backbones so that influenza viral vaccines containing the new strain can be produced. In some embodiments, amplicons are used to produce one or more aspects of the recombinant virus.

Methods of producing recombinant virus using amplicons may include providing in an in vitro system for viral reproduction one or more amplicons coding for at least one viral gene. An amplicon for use in such system may include an RNA polymerase signal, a termination signal and at least a portion of one viral gene or regulatory sequence. The methods can involve using a combination of amplicons and plasmids coding for the viral genes. The methods may include using only amplicons or only plasmids for carrying the viral genes.

A method for producing a virus may include a) providing one or more amplicons and/or plasmids, each of which includes a gene or a portion of a gene of a virus to an in vitro cellular system including a polymerase capable of transcribing the amplicon and/or plasmid viral genes and b) culturing the cells under circumstances that allow for virus production. The method can further include harvesting the virus from the cells or from the cell media.

An amplicon can be produced by, for example, a) amplifying a first fragment, wherein the first fragment includes a fragment of a viral and/or heterologous nucleic acid sequence and a termination sequence; b) amplifying a second fragment, wherein the second fragment includes a fragment of a viral and/or heterologous nucleic acid sequence; c) amplifying a third fragment, wherein the third fragment includes a promoter sequence; and d) combining the three fragments to form an amplicon having a termination sequence, a viral and/or heterologous nucleic acid sequence, and a promoter sequence. A termination sequence may be t1 signal sequence.

For example, initial intermediate constructs including, e.g., one or more of IGIP sequence, signal peptide sequence, linker, cleavage sequence, self-cleaving peptide sequence, reporter, etc. can be synthesized by e.g., Genscript (Piscataway, NJ) in cloning vector such pUC57, and the intermediate fragments subcloned into a suitable reverse genetics vector such as pDP2002 (Perez, et al., Methods Mol Biol, 1602:251-273 (2017)). Subsequently, the PCR fragments encoding the entire HA or NA open reading can be amplified from a suitable source and subcloned into the respective intermediate vectors to form the corresponding IGIP-HA or IGIP-NA vectors encoding the recombinant gene segments.

Transfections can be performed in order to rescue type A or type B LAIV strains expressing IGIP from either the HA or NA segments in the selected, influenza, preferably attenuated influenza, virus backbone.

Viral stocks can generated in, e.g., 10-11-day old specific pathogen free (SPF) eggs. Allantoic fluids can be harvested, centrifuged, aliquoted and stored at e.g., −80° C. Viral stocks can be titrated using any suitable means. For example, tissue culture infectious dose (TCID) or egg infectious dose (EID). Virus titers were calculated using known methods, e.g., Reed and Muench method (Reed, et al., Am. J Hyg., 27:493-497 (1938)).

### III. Vaccine Formulations

Pharmaceutical compositions, vaccine formulations, and dosage units, including 1, 2, 3, 4, 5, or more of the disclosed recombinant virus(es) encoding and expressing an IGIP polypeptide are provided. The compositions may optionally further include other vaccine components including, but not limited to 1, 2, 3, 4, 5, or more other viruses not expressing IGIP (e.g., attenuated backbone viruses) and/or inactivated viruses, viral proteins, adjuvants, etc., and combinations thereof. Any of the compositions may be formulated in a carrier and may include one or more excipients and/or preservatives.

A. Pharmaceutical Carriers

Pharmaceutical compositions containing virus may be for systemic or local administration. Dosage forms for administration by parenteral (intramuscular (IM), intraperitoneal (IP), intravenous (IV), intradermally, or subcutaneous injection (SC)), or transmucosal (nasal, vaginal, pulmonary, or rectal) routes of administration can be formulated.

In some in vivo approaches, the compositions disclosed herein are administered to a subject in a therapeutically effective amount.

For the compositions disclosed herein and nucleic acids encoding the same, appropriate dosage levels for treatment of various conditions in various patients, can be determined by a person skilled in the art, considering the therapeutic context, age, and general health of the recipient. The selected dosage depends upon the desired therapeutic effect, on the route of administration, and on the duration of the treatment desired. Active virus can also be measured in terms of plaque-forming units (PFU) or focus-forming units (FFU, also known as fluorescent focus units). A plaque-forming unit can be defined as areas of cell lysis (CPE) in monolayer cell culture, under overlay conditions, initiated by infection with a single virus particle. The FFU is a variation of the PFU, but instead of relying on cell lysis to detect plaque formation, the FFU is calculated using immunostaining techniques to detect infected host cells and infectious virus particles.

Generally dosage levels of virus may be between 10² and 10¹² FFU. Virus is typically administered in a liquid suspension, in a volume ranging between 100 μl and 5 ml, preferably 0.2 ml to 2 ml, depending on the route of administration. The dose may be administered once or multiple times. Virus delivered locally may be administered in lower doses than virus administered systemically.

For example, FLUMIST® QUADRIVALENT sprayer for intranasal delivery contains a single 0.2 mL dose. Each 0.2 mL dose contains 10^{6.5-7.5} FFU (fluorescent focus units) of live attenuated influenza virus reassortants of each of the four strains: A/Switzerland/3330/2017 (H1N1) (an A/Brisbane/02/2018 (H1N1)pdm09-like virus), A/Kansas/14/2017 (H3N2), B/Phuket/3073/2013 (B/Yamagata/16/88 lineage), and B/Colorado/06/2017 (B/Victoria/2/87 lineage).

FFU or PFU is used in many human vaccines. Dosage may also be expressed in TCID or EID. Most publications with pre-clinical testing of influenza vaccines show doses in fifty-percent Tissue Culture Infective dose (TCID₅₀) or Embryo Infectious Dose (EID₅₀). Typically animals are vaccinated with a dose of 10\hat{ }3-10\hat{ }8 TCID₅₀ or EID⁵⁰ in a volume that ranges between 50 μl-2 ml, depending on the animal species. Thus, such dosages are also contemplated for the disclosed compositions.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amount of nontoxic auxiliary substances such as wetting or emulsifying agents, dyes, pH buffering agents, or preservatives.

Pharmaceutical dosage units of virus are typically administered as a liquid suspension, in a low volume.

Exemplary additives are discussed in more detail below, and should be selected with the understanding that the disclosed att viruses are typically administered as live viruses, and adding agent(s), or amounts thereof, to pharmaceutical composition that could kill or inactive are generally undesirable. For example, the formulations should not include an effective amount of membrane disrupting agents to kill or inactivate the virus.

1. Formulations for Local or Parenteral Administration

In a preferred embodiment, compositions including virus disclosed herein, are administered in an aqueous solution, by parenteral injection. Injection includes, but it not limited to, local, intravenous, intraperitoneal, intramuscular, or subcutaneous. The formulation may also be in the form of a suspension or emulsion. In general, pharmaceutical compositions are provided including effective amounts of virus, and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as anti-oxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. A preferred solution is phosphate buffered saline or sterile saline.

2. Formulations for Mucosal Administration

In some embodiments, the compositions are formulated for mucosal administration, such as through nasal, pulmonary, or buccal delivery.

Mucosal formulations may include one or more agents for enhancing delivery through the nasal mucosa. Agents for enhancing mucosal delivery are known in the art, see for example U.S. Patent Application No. 20090252672 to Eddington, and U.S. Patent Application No. 20090047234 to Touitou. Acceptable agents include, but are not limited to, chelators of calcium (EDTA), inhibitors of nasal enzymes (boro-leucin, aprotinin), inhibitors of muco-ciliar clearance (preservatives), solubilizers of nasal membrane (cyclodextrin, fatty acids, surfactants) and formation of micelles (surfactants such as bile acids, Laureth 9 and taurodehydrofusidate (STDHF)). Compositions may include one or more absorption enhancers, including surfactants, fatty acids, and chitosan derivatives, which can enhance delivery by modulation of the tight junctions (TJ) (B. J. Aungst, et al., J. Pharm. Sci. 89(4):429-442 (2000)). In general, the optimal absorption enhancer should possess the following qualities: its effect should be reversible, it should provide a rapid permeation enhancing effect on the cellular membrane of the mucosa, and it should be non-cytotoxic at the effective concentration level and without deleterious and/or irreversible effects on the cellular membrane, virus membrane, or cytoskeleton of the TJ.

In some embodiments, the virus is administered intranasally.

The carrier formulation, method of administration, or a combination thereof may be similar to that of other intranasal flu vaccines such as FLUMIST® QUADRIVALENT, and thus may be, for example, e.g., 0.2 mL dose is a suspension supplied in a single-dose pre-filled intranasal sprayer. Each 0.2 mL dose contains 10^{6.5-7.5} FFU (fluorescent focus units) of live attenuated influenza virus reassortants of each of the four strains. Each 0.2 mL dose also contains 0.188 mg/dose monosodium glutamate, 2.00 mg/dose hydrolyzed porcine gelatin, 2.42 mg/dose arginine, 13.68 mg/dose sucrose, 2.26 mg/dose dibasic potassium phosphate, and 0.96 mg/dose monobasic potassium phosphate. Each dose contains residual amounts of ovalbumin (<0.024 mcg/dose), and may also contain residual amounts of gentamicin sulfate (<0.015 mcg/mL), and ethylenediaminetetraacetic acid (EDTA) (<0.37 mcg/dose). FluMist Quadrivalent contains no preservatives. To administer the formulation, it is provided in a syringe sprayer suitable for intranasal administration. The tip attached to the sprayer is equipped with a nozzle that produces a fine mist that is primarily deposited in the nose and nasopharynx. FluMist Quadrivalent is a colorless to pale yellow suspension and is clear to slightly cloudy. See, e.g., FLUMIST® QUADRIVALENT package insert.

B. Adjuvants

Adjuvants are also known in the art and can be used in the disclosed compositions and methods, as part of the same or a separate pharmaceutical composition.

Montanide IMS 1313 VG N has been designed specifically for formulation with live vaccine agents and recommended for mucosal or parenteral administration.

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor.

Typically the adjuvant is one that will not kill or inactivate the virus. However, in some embodiments, adjuvants that may kill or inactivate a live virus are used. In some embodiments, the adjuvant is used in an amount that is not effective to kill and/or inactivate the virus. In other embodiments, the adjuvant is used in amount that is effective to kill and/or inactivate the virus, and thus converts the vaccine from a live-virus vaccine to non-live virus vaccine. Such adjuvants may include, but are not limited to, alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene] (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Any adjuvant(s) may also be specifically excluded from the composition.

C. Kits

Dosage units include virus in a pharmaceutically acceptable carrier for shipping and storage and/or administration. Active virus should be shipped and stored using a method consistent with viability such as in cooler containing dry ice so that cells are maintained below 4° C., and preferably below −20° C. VSV virus should not be lyophilized. Components of the kit may be packaged individually and can be sterile. In one embodiment, a pharmaceutically acceptable carrier containing an effective amount of virus is shipped and stored in a sterile vial. The sterile vial may contain enough virus for one or more doses. Virus may be shipped and stored in a volume suitable for administration, or may be provided in a concentrated titer that is diluted prior to administration. In another embodiment, a pharmaceutically acceptable carrier containing an effective amount of virus can be shipped and stored in a syringe.

Typical concentrations of viral particles in the sterile saline, phosphate buffered saline or other suitable media for the virus. Generally, dosage units for live virus should not contain membrane disruptive agents nor should the viral solution be frozen and dried (i.e., lyophilized), which could kill the virus.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. Typically, a larger syringe, pump or catheter will be used to administer virus systemically.

The kits optionally include one or more of the following: bioactive agents, media, excipients and one or more of: a syringe, a bandage, a disinfectant, a local anesthetic, an analgesic agent, surgical thread, scissors, a sterile fluid, and a sterile vessel. Kits for intranasal administration may optionally contain a delivery device for facilitating intranasal delivery, such as a nasal sprayer. The kits are generally provided in a container, e.g., a plastic, cardboard, or metal container suitable for commercial sale. Any of the kits can include instructions for use.

### IV. Methods of Use

Methods of inhibiting and/or preventing influenza viral infections are provided. The live attenuated viruses that can express IGIP disclosed herein, and vaccines including the viruses, can be used to immunize a subject against exposure and infection to an influenza virus, for example, an Influenza A virus, Influenza B, or a combination thereof virus. For example, one or more of the disclosed live attenuated recombinant viruses, e.g., in the form of vaccine or other composition including one or more of the live attenuated recombinant viruses, can be administered to a subject in need thereof for treating, inhibiting, or preventing an influenza infection. The composition can be administered prophylactically to patients or subjects who are at risk of being exposed to an influenza virus, most typically a Type A or Type B influenza virus, or a combination thereof. The composition can also be administered patients or subjects who are newly exposed to the influenza virus or who are at risk of being exposed to an influenza virus, most typically a Type A or Type B influenza virus, or a combination thereof.

The subject can be any animal capable of being infected by an influenza virus. For example, birds including, but not limited to, ducks, chickens, turkeys, and quail, pigs, horses, ferrets, whales, seals, dogs, cats, and rodents can all be infected with various strains of the influenza virus, and can be administered the disclosed compositions. Influenza in birds is often referred to as avian influenza, in pigs as swine influenza, in horses as equine influenza and so on. Influenza in humans is often referred to as the seasonal flu. Waterfowl are important reservoirs for many subtypes of influenza.

Thus, in some embodiments, the compositions are administered to a human subject. In some embodiments, the compositions are administered a non-human animal, optionally, but preferably, wherein administration of the composition to the non-human animals reduces the ability of the animal to maintain or spread one or more strains of virus that can infect humans. In some embodiments, the subject is a ferret, a cat, a dog, a horse, a pig, a cow or a bird. The bird can be an aquatic bird such as a duck, or a domesticated or agricultural bird such as a chicken.

In some embodiments, the recombinant viruses expressing IGIP polypeptide are attenuated relative the wildtype or attenuated backbone virus without the IGIP polypeptide. In some embodiments, expression of the IGIP is effective to increase an immune response against the virus relative to the attenuated backbone virus without the IGIP polypeptide. In some embodiments, the attenuation increases the safety of the virus, preferably while maintaining its ability to be effectively used as a vaccine.

The disclosed compositions are designed to facilitate upregulation of IgA responses on the basis of a live attenuated influenza vaccine. Generally, the IgA antibody is more broadly-neutralizing than the IgG antibody, and IgA is also found predominantly in mucosal surfaces to provide first line protection against influenza viruses. Therefore, the methods can include use of the disclosed compositions as an intranasal flu vaccine, preferably with higher effectiveness against multiple subtypes of flu.

Thus, in some embodiments, the increased immune response is or includes increased antibody titers, particularly IgA and/or IgG antibody titers, relative to the attenuated backbone virus without the IGIP polypeptide. In some embodiments, the higher IgA and/or IgG antibody titer is found in the mucosal tissues, for example the airway, of the subject. In some embodiments, the immune response is or includes an increase in the number and/or activity of nasal-associated lymphoid tissue (NALT). Previous studies have shown that NALT is a site of local long-term specific antibody production (Liang, et al., J Virol, 75(11):5416-20 (2001), Asanuma, et al., J Immunol Methods, 202(2):123-31 (1997)).

In some embodiments, the compositions and methods efficiently stimulate IgA and/IgG at mucosal surfaces and reduce or prevent the virus from reaching target cells. In the most preferred embodiments, the vaccine is one that includes an effective amount of a virus, or combination of the viruses that encode IGIP, IGIP can be secreted from recombinant virus infected cells and is stably maintained in the recombinant influenza virus(es), and the virus(es) retains growth properties in ovo and immunogenicity in vivo.

The efficacy of the attenuated virus can be assessed in various ways well known to the skilled practitioner. For instance, a composition, such as the live attenuated recombinant virus disclosed herein can be determined to be efficacious in treating or inhibiting an influenza infection in a subject by observing that the composition reduces viral load.

In some embodiments, the immune response is against an influenza protein such one or more of H, N, PB2, PB1, PA, NP, M1, M2, NS1, and NEP.

In some embodiments, the influenza virus for which the immune response is desired is one or more influenza A viruses, one or more influenza B viruses, or a combination thereof.

Exemplary influenza A viruses include, but are not limited to, subtypes confirmed in humans such as H1N1, which caused Spanish flu in 1918, and Swine Flu in 2009; H2N2, which caused Asian Flu in 1957; H3N2, which caused Hong Kong Flu in 1968; H5N1, which caused Bird Flu in 2004; H7N7; H1N2, endemic in humans, pigs and birds; H9N2; H7N2; H7N3; H10N7; H7N9, rated in 2018 as having the greatest pandemic potential among the Type A subtypes; and H6N1.

Exemplary influenza B viruses include those of the Victoria lineage (for example, Influenza B/Brisbane, Influenza B/Malaysia) and of the Yamagata lineage (for example, Influenza B/Florida, Influenza B/Phuket, Influenza B/Shanghai, Influenza B/Massachusetts, or Influenza B/Wisconsin).

In some embodiments, the backbone of the attenuated virus is derived from one or more of the influenza viruses against which the immune response is desired for prophylaxis or therapy.

In some embodiments, one or more live attenuated recombinant virus viruses expressing IGIP are administered to a subject as a component in a multivalent vaccine (such as a bivalent, trivalent, or quadrivalent vaccine) directed to inhibiting Influenza A and/or B viruses. A multivalent vaccine of the disclosed compositions and methods may include, for example, 1, 2, 3, 4, or 5 or more different vaccine components, wherein the components are selected from live virus, inactivated virus, viral proteins, etc., and combinations thereof, wherein at least one, and optionally 2, 3, 4, or all 5 of the viruses is/are attenuated recombinant virus(es) encoding and expressing an IGIP polypeptide. In some embodiments, 1, 2, 3, 4, or 5 live viruses of the formulation expression IGIP. Thus, in some embodiments, IGIP-expressing virus(es) are combined with one or more non-IGIP-expressing viruses in a multivalent vaccine formulation.

One influenza A(H1N1), one influenza A(H3N2), and one or two influenza B viruses (depending on the vaccine) are included in each season's influenza vaccines. Getting a flu vaccine can protect against flu viruses that are like the viruses used to make vaccine. Thus, in some embodiments, the disclosed vaccines include components for vaccination against one influenza A(H1N1), one influenza A(H3N2), and one or two influenza B viruses, wherein at least one, and optionally 2, 3, or all 4 of the virus is/are attenuated recombinant virus(es) encoding and expressing an IGIP polypeptide. The remaining vaccination components may be inactivated virus, viral proteins, etc., and combinations thereof.

The disclosed compositions and additional vaccine components and adjuvants can be formulated together in the same admixture, or can be formulated in two or more separate pharmaceutical compositions. Two more different compositions may be administered at the same time, e.g., co-administered, or separately, e.g., hours, days, or weeks apart.

Any of the compositions can be administered as part of vaccine regime including 1, 2, 3, 4, 5, or more administrations of the disclosed compositions, 1, 2, 3, 4, 5, 5, 6, or 7 days, weeks, or months apart.

In a more specific embodiment the regime is a prime-boost regime, 3 weeks apart. The prime-boost can be as live-live or live-inactivated vaccine regime.

The methods are discussed in most detail with respect to influenza, however, it will be appreciated that alternative recombinant viruses encoding IGIP discussed herein can be utilized in methods of inducing immune responses and methods of treating and preventing disease according to the selected recombinant virus background and/or any antigenic protein(s) expressed by the virus. Thus, in some embodiments, the methods are for inducing an immune response against e.g., vaccinia virus, adenovirus, herpes virus, paramyxovirus 5, Newcastle disease virus, measles virus, yellow fever virus, mumps virus, rubella virus, or rotavirus, or a heterologous antigen encoded/expressed by the virus, and/or protecting against a disease caused thereby.

The disclosed compositions and methods can be further understood through the following numbered paragraphs.

1. A nucleic acid comprising a nucleic acid sequence encoding an IgA-inducing protein (IGIP) polypeptide optimally that can increase IgA expression, and a nucleic acid sequence encoding a hemagglutinin (H) or a neuraminidase (N), operably linked to one or more expression control sequences.

2. The nucleic acid of paragraph 1, further comprising a nucleic acid sequence encoding the autologous IGIP signal peptide sequence or a heterologous signal peptide sequence operably linked to the sequence encoding the IGIP polypeptide.

3. The nucleic acid of paragraphs 1 or 2, wherein the IGIP polypeptide comprises the mature form of the IGIP of any one of SEQ ID NOS:1-12, or a functional fragment thereof, or variant thereof with at least 70%, 75%, 80%, 85%, 90%, 95%, or more sequence identity to any one of SEQ ID NOS:1-12.

4. The nucleic acid of any one of paragraphs 1-3, comprising the nucleic acid sequence encoding an H and further comprising a nucleic acid sequence encoding the autologous H signal peptide sequence or a heterologous signal peptide sequence operably linked to the sequence encoding the H.

5. The nucleic acid of paragraph 4, wherein the H is a mature form of an H from an influenza Type A virus.

6. The nucleic acid of paragraph 5, wherein the H is a mature form of any one of H1 to H18.

7. The nucleic acid of paragraph 4, wherein the H is a mature form of an H from an influenza Type B virus.

8. The nucleic acid of any one of paragraphs 1-3, comprising the nucleic acid sequence encoding an N and further comprising a nucleic acid sequence encoding the autologous N signal peptide sequence or a heterologous signal peptide sequence operably linked to the sequence encoding the N.

9. The nucleic acid of paragraph 8, wherein the N is a mature form of an N from an influenza Type A virus.

10. The nucleic acid of paragraph 9, wherein the N is a mature form of any one of N1 to N11.

11. The nucleic acid of paragraph 8, wherein the N is a mature form of an N from an influenza Type B virus.

12. The nucleic acid of any one of paragraphs 1-11, wherein the sequence encoding the IGIP polypeptide and the sequence encoding H or N are separated by a nucleic acid sequence encoding a protease cleavage site, optionally wherein the protease cleavage site is a Furin cleavage site.

13. The nucleic acid of any one of paragraphs 1-12, wherein the sequence encoding the IGIP polypeptide and the sequence encoding H or N are separated by a nucleic acid sequence encoding a self-cleaving peptide, optionally wherein the self-cleaving peptide is 2A self-cleaving peptide, optionally wherein the 2A self-cleaving peptide is selected from

(SEQ ID NO: 16)
EGRGSLLTCGDVEENPGP,
(SEQ ID NO: 17)
ATNFSLLKQAGDVEENPGP,
(SEQ ID NO: 18)
QCTNYALLKLAGDVESNPGP,
and
(SEQ ID NO: 19)
VKQTLNFDLLKLAGDVESNPGP.

14. The nucleic acid of any of paragraphs 1-13, wherein the sequence encoding the IGIP polypeptide and the sequence encoding H or N are separated by a nucleic acid sequence encoding a peptide linker, optionally wherein the peptide linker comprises one or more glycines and one or more serines.

15. The nucleic acid of any one of paragraphs 1-14, wherein the nucleic acid is an influenza genomic RNA segment having the orientation: ‘5 nucleic acid sequence encoding IGIP polypeptide-nucleic acid sequence encoding H or N 3’.

16. The nucleic acid of any one of paragraphs 1-14, wherein the nucleic acid is an influenza genomic NRA segment having the orientation: ‘5 nucleic acid sequence encoding H or N-nucleic acid sequence encoding IGIP polypeptide 3’.

17. The nucleic acid of any one of paragraphs 1-16, further comprising 5′ untranslated region (UTR), 3′ untranslated region (UTR), or a combination thereof.

18. The nucleic acid of paragraph 17, wherein the 5′ UTR, 3′ UTR, or combination thereof from an influenza virus.

19. The nucleic acid of paragraph 18, wherein the nucleic acid encodes H and comprises the 5′ UTR and 3′ UTR of segment 4 of an influenza virus at the 5′ and 3′ ends respectively of the nucleic acid.

20. The nucleic acid of paragraph 18, wherein the nucleic acid encodes N and comprises the 5′ UTR and 3′ UTR of segment 6 of an influenza virus at the 5′ and 3′ ends respectively of the nucleic acid.

21. A nucleic acid comprising the reverse complementary sequence of the nucleic acid of any one of paragraphs 1-20.

22. The nucleic acid of any one of paragraphs 1-21, wherein the nucleic acid is DNA or RNA.

23. The nucleic acid of any one of paragraphs 1-22, wherein the nucleic acid is single stranded or double stranded.

24. The nucleic acid of any one of paragraphs 1-23, wherein the nucleic acid is circular or linear.

25. A vector comprising the nucleic acid of any one of paragraphs 1-14.

26. A viral genomic segment comprising the nucleic acid of any one of paragraphs 1-21.

27. A virus comprising the viral genomic segment of paragraph 26.

28. The virus of paragraph 27, wherein the remaining viral genomic segments are from an influenza virus.

29. The virus of paragraph 28, wherein the influenza virus is a Type A influenza virus.

30. The virus of paragraph 29, comprising the genomic structure:
Segment 1 encoding PB2;
Segment 2 encoding PB1 and optionally PB1-F2;
Segment 3 encoding PA and optionally PA-X;
Segment 4 encoding H;
Segment 5 encoding NP;
Segment 6 encoding N;
Segment 7 encoding M1 and M2; and
Segment 8 encoding NS1 and NEP;
   wherein Segment 4 comprises the nucleic acid sequence of any one of paragraphs 1-23 encoding H or Segment 6 comprises the nucleic acid sequence of any one of paragraphs 1-23 encoding N.

31. The virus of paragraph 28, wherein the influenza virus is a Type B influenza virus.

32. The virus of paragraph 31, comprising the genomic structure:
Segment 1 encoding PB1;
Segment 2 encoding PB2;
Segment 3 encoding PA;
Segment 4 encoding H;
Segment 5 encoding NP;
Segment 6 encoding N and NB;
Segment 7 encoding M1 and BM2; and
Segment 8 encoding NS1 and NEP;
   wherein Segment 4 comprises the nucleic acid sequence of any one of paragraphs 1-23 encoding H or Segment 6 comprises the nucleic acid sequence of any one of paragraphs 1-23 encoding N.

33. The virus of any one of paragraphs 28-32, wherein the influenza virus is an attenuated influenza virus.

34. The virus of paragraph 33, wherein the attenuated virus comprises a reassorted genome, temperature sensitive mutation(s), NS1 truncation, elastase-dependency, rearranged genome, or a combination thereof.

35. The virus of paragraphs 33 or 34, wherein the attenuated influenza virus is OH/04 att, cold-adapted Leningrad (ca/LEN), or B/Bris att.

36. A pharmaceutical composition comprising the live virus of any one of paragraphs 27-35 in a pharmaceutically acceptable carrier for administration to a subject.

37. The pharmaceutical composition of paragraph 36, further comprising one or more additional influenza viruses.

38. The pharmaceutical composition of paragraph 37, wherein the one or more additional influenza viruses are attenuated viruses.

39. The pharmaceutical composition of any one of paragraphs 36-38 comprising one or more H1N1, H2N2, H3N2, H5N1, H7N7, H1N2, H9N2, H7N2, H7N3, H10N7, H7N9, and/or H6N1 influenza A subtypes.

40. The pharmaceutical composition of any one of paragraphs 36-39 comprising one or more influenza B viruses.

41. The pharmaceutical composition of any one of paragraphs 36-40, further comprising an adjuvant.

42. The pharmaceutical composition of any one of paragraphs 36-41 formulated for intradermal or intramuscular injection

43. The pharmaceutical composition of any one of paragraphs 36-41 formulated for intranasal delivery.

44. The pharmaceutical composition of any one of paragraphs 36-43 comprising an effective amount of influenza virus(es) in an effective amount to induce an immune response against influenza virus in a subject.

45. The pharmaceutical composition of paragraph 44, wherein the immune response comprises an increase in production of IgA and/or IgG antibodies against influenza virus.

46. A method of inducing or increasing an immune response against influenza virus comprising administering a subject in need thereof an effective amount of the pharmaceutical composition of any one of paragraphs 36-45 to induce or increase an immune response against the influenza virus(es) in the subject.

47. The method of paragraph 46, wherein the immune response provides a preventative or therapeutic effect against infection by influenza virus(es).

48. The method of paragraphs 46 or 47, wherein the pharmaceutical composition is administered by injection or intranasal delivery.

49. The method of paragraphs 46-48, wherein the subject is selected from humans, birds, pigs, horses, ferrets, whales, seals, dogs, cats, and rodents.

50. The method of any one of paragraphs 46-49 comprising repeating the administration one or more times 1, 2, 3, 4, 5, 6, 7, 8, 9, or 10 days, weeks, or months apart.

51. A recombinant virus comprising a genome encoding an IgA-inducing protein (IGIP) polypeptide that can increase IgA and/or IgG expression when expressed in a cell infected by the virus.

52. The recombinant virus of paragraph 51, wherein the virus is a recombinant influenza virus, vaccinia virus, adenovirus, herpes virus, paramyxovirus 5, Newcastle disease virus, measles virus, yellow fever virus, mumps virus, rubella virus, or rotavirus.

53. A pharmaceutical composition comprising an effective amount of the recombinant virus of paragraphs 51 or 52 to induce an immune response against the virus in a subject in need thereof, preferably wherein the immune response against the virus is greater than the immune response induced with the virus in the absence of expression of IGIP.

54. The pharmaceutical composition of paragraph 53, wherein the recombinant virus is a live virus.

55. A method of inducing an immune response in a subject in need thereof comprising administering the subject the pharmaceutical compositions of paragraphs 53 or 54.

### EXAMPLES

Cáceres, et al., “Development of a Novel Live Attenuated Influenza A Virus Vaccine Encoding the IgA-Inducing Protein,” Vaccines, 9(703), 21 pages (2021). doi.org/10.3390/vaccines9070703, including all of the Supplemental Materials associate (mdpi.com/article/10.3390/vaccines9070703/s1) is specifically incorporated by reference herein in its entirety.

### Example 1: Design and Production of IGIP-LAIVs

### Materials and Methods

Cells

Madin-Darby canine kidney (MDCK) and human embryonic kidney 293T cells (HEK293T) were a kind gift from Robert Webster (St Jude Children's Research Hospital, Memphis, TN, USA). MDCK STAT1 KO cells (CCL-34-VHG) were purchased from ATCC. Cells were maintained in Dulbecco's Modified Eagles Medium (DMEM, Sigma-Aldrich, St Louis, MO, USA) containing 10% fetal bovine serum (FBS, Sigma-Aldrich, St Louis, MO, USA), 1% antibiotic/antimycotic (AB, Sigma-Aldrich, St Louis, MO, USA) and 1% L-Glutamine (Sigma-Aldrich, St Louis, MO, USA). Cells were cultured at 37° C. under 5% CO₂.

Generation of Plasmids Encoding the IGIP Flanked by Segment 4 (HA) Sequences.

The IGIP protein is highly conserved among mammals and expressed by antigen-presenting dendritic cells (DCs) in the intestinal tract as a 47-54 aa protein. IGIP is thought to play a role in the regulation of IgA expression in the intestinal tract. The C-terminal 24 aa in IGIP correspond to the mature active peptide, whereas the N-terminal ˜30 aa correspond to the signal peptide region (FIG. 1C).

Plasmid constructs were designed to carry, in the order 5′-3′, the 5′-UTR of segment 4, the signal peptide of H1 HA (A/California/04/09 (Ca/04) (H1N1)), the IGIP mature peptide sequence, the G4S linker, the furin-recognition cleavage site, the Thosea assigna virus 2A protein sequence, the signal peptide of Gaussia luciferase (Gluc), a cloning spacer and the 3′-UTR of segment 4. Three such constructs were produced for 1) H1 and 2) H3 and 3) influenza B (IBV) HA sequences. These initial intermediate constructs were synthesized by Genscript (Piscataway, NJ) in a pUC57 cloning vector.

The intermediate fragments were subcloned into the reverse genetics vector pDP2002 (Perez, et al., Methods Mol Biol, 1602:251-273 (2017), Pena, et al., J Virol., 85, 456-469 (2011), Wan, et al., J Virol., 92 (2018)) generating an intermediate plasmid pDP2002-IGIP). Subsequently, the PCR fragments encoding the entire HA open reading frame from the strains A/California/04/2009 (H1N1)(Ca/04), A/turkey/Ohio/313053/2004 (H3N2)(OH/04), and B/Brisbane/60/2008 (B/Bris) (Wan, et al., J Virol, 92(21) (2018), Ye, et al., PLoS Pathog, 6(10): e1001145 (2010), Santos, J Virol, 91(12) (2017) were subcloned into the respective intermediate vectors to the corresponding IGIP-HA chimeric gene segments, generating the plasmid pDP2002-IGIP-H1. The pDP2002-IGIP-H1 sequence was confirmed by Sanger sequencing (Psomagen, Rockville, MD, USA).

See, e.g., FIG. 1B.

Generation of Plasmids Encoding the IGIP Flanked by Segment 6 (NA) sequences.

Three plasmid vectors (N1, N2 and NB) in the pUC57 backbones were produced by Genscript encoding the 5′-UTR of segment 6 followed by a cloning spacer downstream the 5′ UTR, the G4S linker, the furin-recognition cleavage site, the Thosea assigna virus 2A protein sequence, the Gluc signal peptide, the IGIP mature peptide sequence and the 3′-UTR of segment 6. The cassette was cloned into pDP2002 and the entire open reading from either the N1, N2, or influenza B NA was amplified and cloned into the corresponding spacer region.

See, e.g., FIG. 1C.

Generation of LAIV Candidates Expressing IGIP.

Transfections were performed in order to rescue type A LAIV strains expressing IGIP from either the HA or NA segments in either the OH/04 att backbone (Pena, et al., J Virol, 85(1):456-69 (2011), Loving, et al., Vaccine, 30(40):5830-8 (2012), Gauger, Virology, 471-473:93-104 (2014)) or the cold-adapted Leningrad backbone (ca/LEN) (Ghendon, Infect Immun, 44(3):730-3 (1984), Isakova-Sivak, et al., Vaccines (Basel), 7(3) (2019)). Likewise, transfections were performed to obtain type B LAIV strains with or without IGIP in the backbone of B/Bris att (Wan, et al., J Virol, 92(21) (2018), Santos, J Virol, 91(12) (2017)).

For example, pDP2002-IGIP-H1 or the pDPHA-H1 (Ca/04) wild type plasmids were transfected with the 6 plasmids corresponding to the OH/04 attenuated-temperature sensitive ([ts+HA tag=att]) backbone previously described (Wan, et al., J. Virol., 92 (2018)) or the cold adapted Leningrad backbone (caLen) (Isakova-Sivak, Virology, 412, 297-305 (2011)). In both cases, a plasmid expressing the Neuraminidase (NA) of Ca/04 was used. Co-cultures of 9×10⁵ HEK293T and 1.5×10⁵ MDCK cells were seeded per well in a 6-well plate. The following day, 1 μg of each plasmid was mixed with 18 μL of TransIT-LT1 transfection reagent (Mirus Bio LLC, Madison, WI, USA). The mixture was incubated for 45 min and then used to overlay the 293T/MDCK cells overnight. The next day, the transfection mixture was replaced with fresh Opti-MEM media containing 1% AB (Life Technologies, Carlsbad, CA, USA) and 24 h post-transfection, the media was supplemented with 1 μg/mL of tosylsulfonyl phenylalanyl chloromethyl ketone (TPCK) treated-trypsin (Worthington Biochemicals, Lakewood, NJ, USA). Viral stocks were generated in 10-day-old specific pathogen free (SPF) eggs. Allantoic fluids were harvested at 48 h post-infection (hpi), centrifuged, aliquoted and stored at −80° C. Viruses were titrated by tissue culture infectious dose 50 (TCID₅₀) and virus titers were established by the Reed and Muench method (Reed & Muench, Am. J. Epidemiol., 27, 493-497 (1938)). Viral sequences were confirmed by next generation sequencing and sanger sequencing as previously described (Ferreri, et al., Ecol. Evol., 9, 6534-6546 (2019)).

Viral Stock Preparation, Maintenance, and Analysis

Virus stocks were generated in 10-11-day old specific pathogen free (SPF) eggs. Allantoic fluids were harvested at 48 hours post-inoculation (hpi), centrifuged, aliquoted and stored at −80° C. Type A LAIVs were titrated by tissue culture infectious dose 50 (TCID₅₀), whereas type B LAIVs were titrated by egg infectious dose 50 (EID₅₀). Virus titers were calculated by the Reed and Muench method (Reed, et al., Am. J. Hyg., 27:493-497 (1938)).

In Vitro Growth Kinetics

Confluent monolayers of MDCK or MDCK STAT1 KO cells were inoculated at a multiplicity of infection (MOI) of 0.01 for each virus. Plates were incubated 15 min at 4° C. and then 45 min at 35° C. Subsequently, the virus inoculum was removed, and the cells were washed twice with 1 mL of phosphate-buffered saline (PBS). Opti-MEM I (Life Technologies, Carlsbad, CA, USA), containing TPCK-trypsin (Worthington Biochemicals, Lakewood, NJ, USA) and antibiotic-antimycotic solution (Sigma-Aldrich, St. Louis, MO, USA), was then added to the cells (Opti-MEM-AB+TPCK). At the indicated time points, tissue culture supernatant from inoculated cells was collected for virus titer quantification. Virus RNA from tissue culture supernatant was isolated using the MagMAX-96 AI/ND viral RNA isolation kit (Thermo Fisher Scientific, Waltham, MA, USA). Virus titers were determined using a real-time reverse transcriptase PCR (RT-qPCR) assay based on the influenza A matrix gene. The RT-qPCR was performed in a QuantStudio 3 (Applied Biosystem, Foster City, CA, USA) using qScript™ XLT One-Step RT-qPCR ToughMix®, QuantaBio (ThermoFisher). A standard curve was generated using 10-fold serial dilutions from a virus stock of known titer to correlate quantitative PCR (qPCR) crossing-point (Cp) values with virus titers, as previously described (Santos, et al., J. Virol., 93 (2019)). Virus titers were expressed as log 10 TCID50/mL equivalents.

Graphs/Statistical Analyses

All data analyses and graphs were performed using GraphPad Prism software version 9 (GraphPad Software Inc., San Diego, CA, USA). A one-way ANOVA was performed. A P value below 0.05 was considered significant.

Results

Experiments were designed to create live attenuated influenza virus (LAIV) vaccines expressing the IgA-inducing protein (IGIP)—also referred to herein as IGIP-LAIVs, and test the ability of IGIP to be secreted from IGIP-LAIV infected cells, be stably maintained in recombinant influenza viruses, and retain growth properties in ovo and immunogenicity in vivo.

A stable and efficacious alternative LAIV strategy for IAV was previously developed carrying a ts mutation in the PB2 ORF, and ts mutations and a C-terminal epitope tag in the PB1 ORF [ts+HA tag=att].

The att strategy share some ts mutations in common with the MDV-A caA/Ann Arbor (Chan, et al, Virology, 380, 304-311 (2008)) and its safety, immunogenicity, and efficacy has been demonstrated in Balb/c mice and pigs (Pena, et al., J Virol., 85, 456-469 (2011), Chan, et al., Virology, 380, 304-311 (2008), Gauger, et al., Virology, 471-473, 93-104 (2014), Loving, et al., J Virol., 87, 9895-9903 (2013)). New viruses were designed to improve the safety profile of the att candidate and test if IGIP could better stimulate protective antibody responses against IAV. To further these studies, the IGIP was also tested in the caLen backbone, which is currently approved for human use. The C-terminal 24 aa in IGIP correspond to the mature active peptide, whereas the N-terminal ˜30 aa correspond to the signal peptide region (FIG. 1C). The swine IGIP mature peptide sequence, was cloned as an N-terminal tag of the HA ORF in segment 4. Specifically, IGIP was cloned downstream of the signal peptide region of the H1 HA of A/California/04/2009 (H1N1) (Ca/04) virus, followed by the G4S linker peptide, an artificial furin cleavage site, the Thosea assigna virus 2A protease sequence, the signal peptide region of Gaussia luciferase and then the mature HA ORF (FIG. 1B). The reverse genetics (RG) plasmid carrying the modified IGIP-H1 HA segment was combined with the RG plasmid encoding the N1 NA of Ca/04 and 6 RG plasmids encoding the backbone of either OH/04att or caLen. As a control, isogenic viruses carrying the wild type H1 HA of Ca04 were prepared.

Successful virus rescues were obtained for the IGIP-H1, IGIP-H3, N1-IGIP and N2-IGIP segments in the OH/04 att backbone. Only the IGIP-H1 segment rescued in the ca/LEN backbone. For the type B LAIV, only the IGIP-HA chimeric construct was rescued. At least 2 independent attempts were performed to rescue the IGIP-H3, N1-IGIP and N2-IGIP in the ca/LEN backbone and the IGIP-NA in the B/Bris att backbone but those were unsuccessful. All other isogenic controls without the IGIP modifications were rescued. See Tables 2 and 3.

TABLE 2
Type/subtype of IGIP viruses produced and attempted. Titers of
type A virus vaccines candidates are shown in TCID₅₀/ml, whereas
those of type B virus vaccine candidates are shown in EID₅₀/ml.

| Virus | Backbone | Titers |
|---|---|---|
| att (H1N1) | OH/04 att | 1 × 10⁸ |
| att (H3N2) | OH/04 att | 1 × 10⁸ |
| IGIP-H1-att (H1N1) | OH/04 att | 5 × 10⁸ |
| IGIP-H3-att (H3N2) | OH/04 alt | 2 × 10⁷ |
| N1-IGIP-att (H1N1) | OH/04 att | 2 × 10⁶ |
| N2-IGIP-att (H3N2) | OH/04 att | 2 × 10⁶ |
| ca/Len (H1N1) | ca/Len | 2 × 10⁷ |
| ca/Len (H3N2) | ca/Len | 2 × 10⁷ |
| IGIP-H1-ca/LEN (H1N1) | ca/Len | 1 × 10⁸ |
| IGIP-H3-ca/LEN (H3N2) | ca/Len | Did not rescue |
| N1-IGIP-ca/LEN (H1N1) | ca/Len | Did not rescue |
| N2-IGIP-ca/LEN (H3N2) | ca/Len | Did not rescue |
| IDV-att | B/Bris-att | 2.3 × 10⁸ |
| IGIP-HA-IBV-att | B/Bris-att | 4.2 × 10⁷ |
| NA-IGIP-IBV-att | B/Bris-att | Did not rescue |

TABLE 3
Viral titers of the different viruses.

| | | Titer | IGIP-H1 Segment 4 Stability | | | |
|---|---|---|---|---|---|---|
| HA Gene | Backbone | TCID₅₀/mL | Segment | Modification | E1 (NGS) ^{a} | E5 (Sanger) ^{b} |
| IGIP-H1 | OH/04att | 5 × 10⁶ | HA | HA SP | No change | t58c⁶⁶⁵⁰⁻⁵⁴⁰⁰(L9P)¹ [symbol] |
| | | | | IGIP | No change | t86c^{S} [symbol] |
| | | | | G4S [symbol] | No change | No change |
| | | | | Furin CS | No change | No change |
| | | | | Tav2A | No change | No change |
| | | | | G1orS [symbol] | No change | No change |
| | | | PB2 | S265 (g823) | No change | No change |
| | | | PB1 | E391(g1195, g1197) | No change | No change |
| | | | | GS82(g1766) | No change | No change |
| | | | | T661(a2005, g2007) | No change | No change |
| | | | | HA tag | No change | No change |
| | | | Other | N/A ^{d} | No change | Not performed |
| | | | segments | | | |
| H2 | OH/04att | 2.32 × 10⁷ | N/A | N/A | N/A | N/A |
| IGIP-H2 | caLen | 1 × 10⁴ | N/A | N/A | N/A | N/A |
| H2 | caLen | 2 × 10⁷ | N/A | N/A | N/A | N/A |

[symbol]

indicates data missing or illegible when filed

Passage 1 of the virus in SPF eggs (E1) was sequenced by next generation sequencing using Illumina MiSeq. a E1 virus was sequenced by NGS b E5 virus was sequenced by Sanger sequencing of full-length HA, PB2 and PB1 RT-PCR fragments with appropriate primers (list available upon request). c L9P mutation reduces predicted signal peptide cleavability at the “ . . . ANA-GN . . . ” cleavage site from >0.9 to 0.8765 based on SignalP v. 5.0 predictive tool (Armenteros, et al., Nat. Biotechnol., 37, 420-423 (2019)). d N/A, not applicable.

Analysis by segment specific PCR (type A LAIVs) or multi-segment PCR (type B LAIVs) revealed differences in segment size depending on presence or absence of IGIP modification.

Sanger and NGS (MiSeq Illumina, (Ferreri, et al., Ecol Evol, 9(11) p. 6534-6546 (2019))) sequencing of virus stocks showed the presence of the expected IGIP fragment in either the HA or NA segments.

Virus titers results revealed that the IGIP modification on HA in the OH/04 att backbone caused drops of 1-1.5 log 10 compared to the non-IGIP isogenic controls; but similar to titers obtained with the non-IGIP ca/LEN controls. The NA-IGIP modification in the OH/04 att backbone dropped virus titers ˜2 log 10 compared to isogenic controls.

Although IGIP-HA (H1N1) viruses were rescued in both attenuated backgrounds (Table 3), the IGIP-H1caLen virus grew poorly in both MDCK cells and in eggs in comparison to the isogenic H1caLen without IGIP, and, therefore, it was not included in subsequent analyses. In contrast, the IGIP-H1att grew to titers such as the isogenic H1att virus (Table 3), and both showed similar growth kinetics at 35° C. in MDCK cells, as well as MDCK STAT1 KO cells (FIG. 1D). IGIP-HA-LAIV with a modified H3 HA gene segment that carries IGIP achieves virus titers similar to the unmodified LAIV at the permissive temperature (35° C.). The incorporation of the IGIP-HA segment showed delayed growth at 37° C. and enhanced growth restriction at non-permissive temperatures of 39° C. and 41° C. See FIGS. 1E-1H. Importantly, serial passages of the IGIP-H1att virus showed that the modified HA segment was maintained for at least five passages in SPF eggs and MDCK cells (Table 3).

NA-IGIP chimeric constructs with extended 3′ UTR may improve segment 6 packaging into viruses, which may improve virus yields.

In an alternative construct, the signal peptide in the H3 IGIP-HA chimera was modified to include two additional amino acids at the cleavage site, as bioinformatics analysis indicated improved cleavage would result with the incorporation of two additional amino acids at the C-terminus of the signal peptide sequence before the start of the IGIP sequence. Experiments show this virus rescues and grows.

As mentioned above, the IGIP-H1 was able to rescue in the ca/LEN backbone; however, the virus showed poor yields (104 TCID₅₀/ml). The poor growth of the IGIP-H1 ca/LEN virus and the lack of rescue of the other 3 chimeric constructs are perhaps due to over attenuation in which the ca/LEN appears to not tolerate additional modifications. The IGIP-HA modification in the B/Bris att backbone caused a small drop (<1 log 10) in titer compared to the isogenic B/Bris att control.

### Example 2: IGIP-LAIV is Safe and Efficacious Against Influenza In Vivo

### Materials and Methods

Safety and Efficacy

A safety/efficacy study utilized the following vaccine candidates IGIP-H1 att (H1N1) and N1-IGIP att (H1N1) and compared them to the non-IGIP att (H1N1) virus and the non-IGIP ca/LEN (H1N1) viruses. Additional control groups included non-vaccinated/challenge and non-vaccinated/non-challenged (only the most relevant groups are shown). Male and females DBA/2J mice (5 to 6 weeks-old, n=16/group, ½ female) were used.

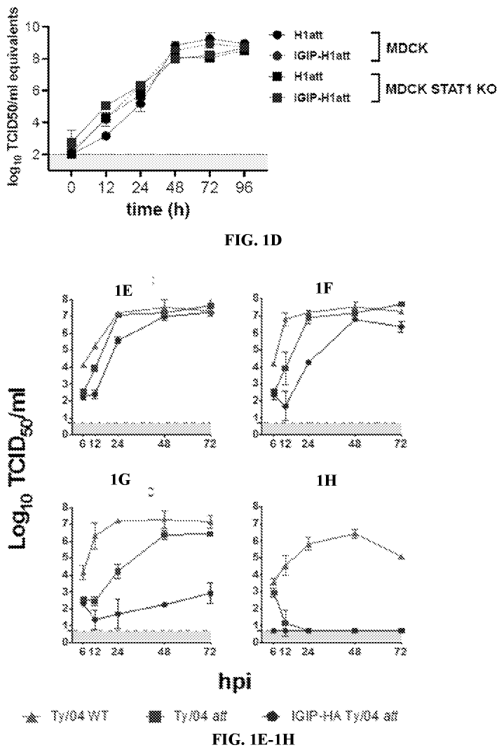

For prime and boost, mice received intranasally a dose of 1×10⁵ TCID₅₀/mouse (50 μl dose) of the corresponding vaccine, 21 days apart. Clinical signs of disease consistent with influenza infection were monitored for two weeks after prime and after boost. See FIGS. 2A and 2B.

For influenza B studies, following the prime-boost design described above, DBA/2J mice (n=12/group, 12 female) were vaccinated intranasally with 10⁶ EID₅₀/mouse (50 μl dose) of either the IGIP-HA IBV att virus or the non-IGIP IBV att control virus.

Three weeks after boost, a subset of mice from each group were bled to assess seroconversion, and the rest challenged intranasally with 10⁷ EID₅₀/mouse (˜10 MLD₅₀) of the B/Brisbane/60/2008 PB2 F406Y virus (Santos, J Virol, 91(12) (2017) or 1×10⁶ TCID50/mouse (˜10,000 mouse lethal dose 50) of A/California/04/2009 (H1N1) (Ca/04) mouse-adapted strain as previously described (Ye, et al., PLoS Pathog., 6, el 001145 (2010)). Clinical signs, bodyweight changes and mortality were monitored daily. Mice that lost ≥25% of their initial body weight (a score of 3 or higher on a 3-point scale of disease severity) were humanely euthanized. To obtain serum samples before euthanasia, mice were bled from the submandibular vein as previously described (Golde, et al., Lab Anim., 34, 39-43 (2005)). Two weeks after challenge, all survivors were bled, euthanized, and tissue samples collected.

Virus Titration

Nasal turbinates and lungs homogenates collected from mice at 5 dpc were generated using the Tissue Lyzer II (Qiagen, Hilden, Germany). Briefly, 1 mL of PBS-AB was added to each tissue together with Tungsten carbide 3 mm beads (Qiagen). Samples were homogenized for 15 min and then centrifuged at 15,000 g for 10 min. Supernatants were collected, aliquoted and stored at −80° C. until further analysis. Samples were titrated by TCID₅₀ and virus titers were established by the Reed and Muench method (Reed & Muench, Am. J. Epidemiol., 27, 493-497 (1938)).

Histopathology Examination

Lungs were collected from a representative number of mice (n=4) in each group at 5 dpc for histopathological examination. Tissues were placed in 10% neutral-buffered formalin (NBF), fixed for at least 72 h, paraffin embedded and processed for routine histopathology with hematoxylin and eosin staining (HE). Lesions were subjectively scored by a pathologist blinded to the study as: none (0), mild; ≤10% (1), mild to moderate; 11-25% (2), moderate; 26-40% (3), moderate to severe; 41-60% (4) and ≥60% (5) severe, based on lesion severity and extent of inflammation. Features considered for the scoring were the following: bronchitis/bronchiolitis, alveolitis, pleuritis and vasculitis, type of inflammatory infiltrate, presence and extent of necrosis, hemorrhage, edema (interstitial and/or alveolar), fibrin/hyaline membranes, pneumocyte type 2 hypertrophy and hyperplasia and pleural mesothelial hyperplasia. For immunohistochemistry (IHC) against I-V, a polyclonal antibody anti-IAV H1N1 (Meridian Life Science; dilution 1/1500) was used. The staining was used to estimate the intensity of viral antigens. Staining intensity and distribution were subjectively scored by a pathologist blinded to the study using a scale from none (0) to large/highest positivity (5).

### Results

Experiments were designed to demonstrate that the IGIP-LAIV vaccines are safe and efficacious against influenza in vivo and to show stimulation of enhanced IgA levels compared to controls.

To evaluate the IGIP vaccine candidates, their safety, immunogenicity and protection efficacy were tested against high dose influenza challenge in DBA/2J mice as previously described (Wan, et al., J Virol, 92(21) (2018)). The DBA/2J strain was chosen due to its higher susceptibility to influenza compared to the Balb/c mouse strain (Pica, et al., J Virol., 85, 12825-12829 (2011), Srivastava, et al., PLoS One, 4(3): p. e4857 (2009)), to more thoroughly assess the safety profiles of IGIP- and non-IGIP-LAIV candidates. A prime-boost vaccination strategy, 21 days apart, was used.

DBA/2J mice showed substantial weight loss and succumbed to influenza infection (8/8 females, 7/8 males) after inoculation with the non-IGIP att (H1N1) virus. This observation contrasts a similar previous study performed in female Balb/c mice (FIG. 2 in Pena, et al., J Virol, 85(1):456-69 (2011)), in which such virus was completely attenuated causing no disease signs and no body weight loss in inoculated animals.

Although unanticipated, the outcome of the non-IGIP att (H1N1) virus inoculated DBA/2J mice is consistent with the notion of their higher susceptibility to influenza viruses (Pica, et al., J Virol, 85(23):12825-9 (2011)).

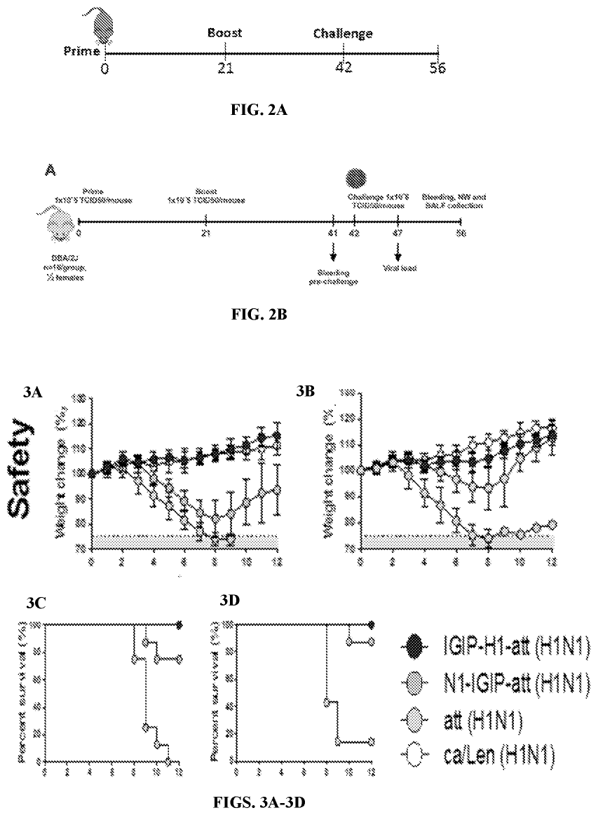

The incorporation of the IGIP modification in HA led to substantial attenuation, whereas such modification in NA caused partial attenuation in both female and male mice after prime (although still significant morbidity in female mice). The level of attenuation of the IGIP-H1 att (H1N1) was similar to the ca/LEN (H1N1) control after prime. Boost immunizations caused no noticeable disease signs. In sum, incorporation of the IGIP modification in the HA (segment 4) significantly improved the safety profile of the OH/04 att, similar to the control ca/Leningrad (ca/Len) backbone strain in female and male DBA/2J mice. Incorporation of IGIP modification in the NA (segment 6), led to partial attenuation but still produced significant body weight loss in female DBA/2J mice. See FIGS. 3A-3D.

See also FIG. 3E-3F. The DBA/2 J mice in the H1att group showed weight loss starting on 4 dpi with rapid deterioration of clinical signs and mortality between 8-10 dpi (1 survivor out of 16, FIG. 3E, 3F). In contrast, no clinical signs, negligible weight changes, and no mortality were observed in mice that were inoculated with IGIP-H1att or the H1caLen groups (FIG. 3E, 3F).

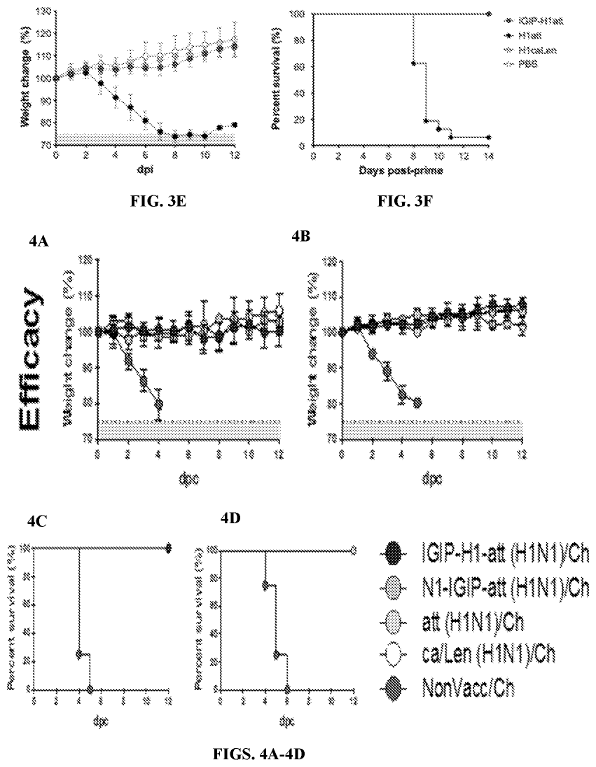

In order to test the efficacy of the IGIP-H1att vaccine, vaccination in a prime-boost strategy 3 weeks apart was used (FIG. 2B). On day 21 post-boost (42 days post-vaccination), a subset of mice from each group were bled to assess seroconversion whereas the rest were challenged intranasally with 1×10⁶ TCID₅₀/mouse (˜10,000 mouse lethal dose 50; MDL₅₀) of the Ca/04 (H1N1) mouse adapted strain (Ye, et al., PLoS Pathog, 6(10): e1001145 (2010)). Results show complete protection from clinical signs and 100% survival in mice that received either the IGIP-H1 att (H1N1) vaccine or the N1-IGIP att (H1N1) vaccine, comparable to the ca/Len control virus vaccine. See FIGS. 4A-4F).

Consistent with these observations, virus shedding below limit of detection were observed in samples from lungs and nasal turbinates (NT) collected from a subset of mice at 5 dpc from both vaccinated/challenge groups, but not in the mock-vaccinated/challenge group (FIG. 4G, 4H). Histopathological examination in lungs showed more severe lesions in mice from the mock-vaccinated/challenge group compared to the other groups (Table 4). These were characterized by moderate to severe random areas of necrosis, characterized by discontinuous alveolar septa replaced by brightly eosinophilic, fibrillar material (fibrin) admixed with hemorrhage, alveolar edema, karyorrhectic cellular debris, viable and degenerate neutrophils and foamy macrophages. Bronchial epithelium was occasionally affected with attenuation, deciliation and single cell necrosis. In contrast, vaccinated/challenge groups presented similar patterns of lesions with mild or mild-moderate numbers of lymphocytes, plasma cells and lesser neutrophils and macrophages expanding peribronchiolar and perivascular interstitium. Bronchial epithelium was minimally affected by deciliation, and single cell drop out. Immunohistochemical staining against IAV antigens was detected only in lungs from the mock-vaccinated/challenge group (Table 4). This was present within the nucleus and cytoplasm of bronchial epithelial cells, alveolar macrophages, pneumocytes and within necrotic cellular debris. No presence of IAV antigens were observed in any of the vaccinated groups and the negative control. These observations indicate that the IGIP-H1att is at least as effective as the H1caLen virus in protecting mice against aggressive challenge with a homologous IAV.

TABLE 4
Histopathological examination (HE) and immunohistochemistry
against IAV scores at 5 dpc in lungs.

| Group | Virus Challenge? | HE | IHC |
|---|---|---|---|
| Mock-vaccinated | Yes | 4-4-4-4 | 3-3-3-3 |
| IGIP-H1att | Yes | 1-0-1-1 | 0-0-0-0 |
| H1-caLen | Yes | 1-2-0-1 | 0-0-0-0 |
| PBS | No | 0-0-0-0 | 0-0-0-0 |

A dash (-) separates the score of each individual mouse (n = 4/group, ½ females).

In sum, regardless of the vaccine received, vaccinated mice showed no signs of disease and were completely protected from high dose virus challenge.

In a separate study, type A IGIP-LAIV candidates were evaluated in pigs for safety and efficacy profiles against high dose influenza heterologous challenge, and to investigate if the IGIP strategy will be beneficial in, e.g., the context of bivalent influenza vaccines. A prime-boost vaccination strategy, 14 days apart, was used, and IGIP vaccines contained both IGIP-H1-att and IGIP-H3-att. The incorporation of the IGIP modification in HA does not alter the attenuation properties of the IAV att backbone in swine, and the vaccine conferred similar safety profile to the non-IGIP att control vaccine.

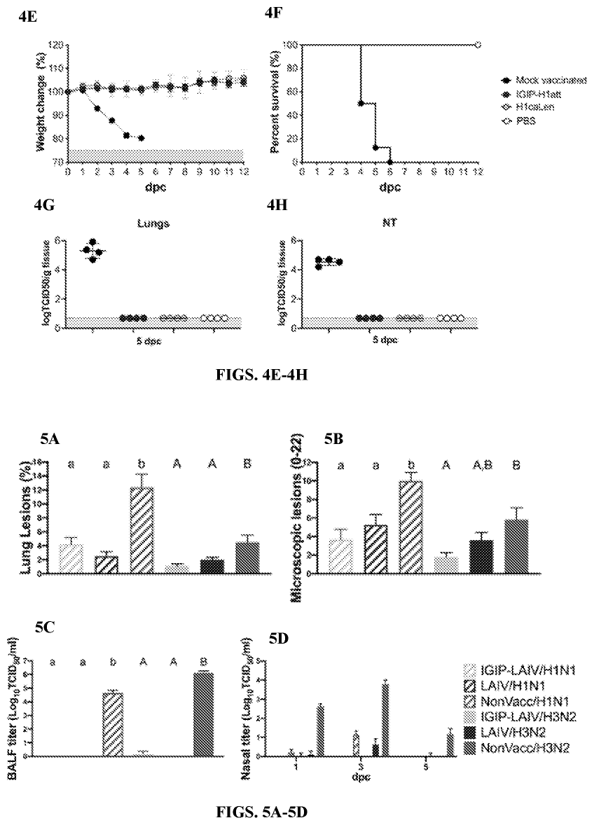

Three weeks after boost (35 days post-vaccination), pigs were challenged intranasally and intratracheally with 3×10⁶ TCID₅₀/pig of an endemic HIN1 or an endemic H3N2, both antigenically distinct from the vaccine antigens. Results show efficacious protection from lung lesions, and almost complete protection against viral replication and shedding, with only few vaccinated animals showing detectable viral titers in the lungs and in nasal samples. In contrast, all animals in the non-vaccinated challenge control groups (NonVacc/H1N1 and NonVacc/H3N2) showed high titers in the lungs and nasal samples, and a significantly higher percentage of the lungs affected with lesions. See FIGS. 5A-5D.

Overall, these results demonstrate that similarly to the type A IGIP-LAIV candidates in mice, the type A IGIP-LAIV candidate virus is safe and confers protection of pigs against heterologous challenge and remains a viable vaccine candidate.

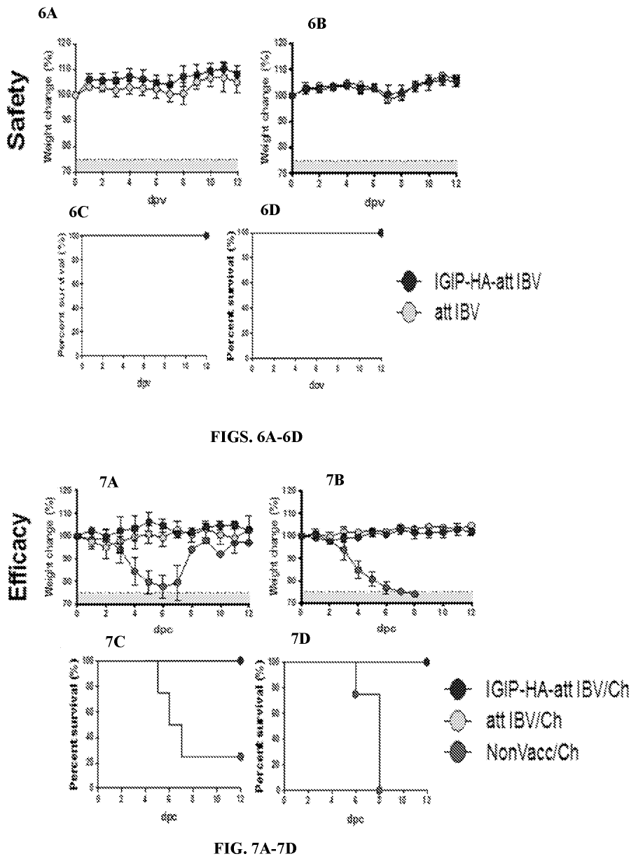

The studies were also expanded into influenza B virus to investigate if the IGIP strategy will be beneficial in, e.g., the context of quadrivalent influenza vaccines. As observed previously with female DBA/2J mice, the IBV att vaccine is also attenuated in male DBA/2J mice. The IGIP modification in HA of, e.g., B Bris strain, does not alter the attenuation properties of the IBV att backbone. FIGS. 6A-6D.

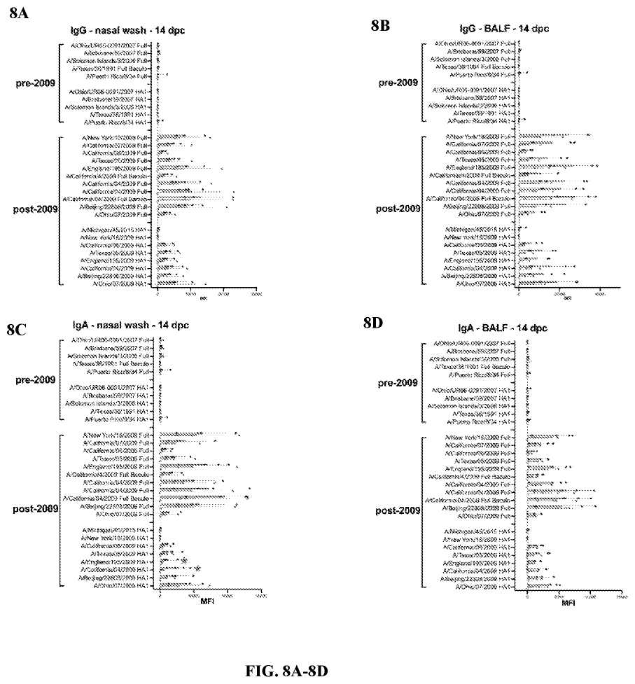

Three weeks after boost, a subset of mice from each group were bled to assess seroconversion, and the rest challenged intranasally with 10⁷ EID₅₀/mouse (˜10 MLD₅₀) of the B/Brisbane/60/2008 PB2 F406Y virus (Santos, J Virol, 91(12) (2017)). This study revealed that the IGIP-HA IBV att vaccine candidate is safe and efficacious against influenza B virus challenge. In contrast, in the non-vaccinated/challenge control group, 4/4 males and 3/4 females succumbed between days 7 and 8 post-challenge, respectively. FIGS. 7A-7D. In sum, results show complete protection from clinical signs and 100% survival in mice that received the IGIP-HA-att IBV vaccine with a performance comparable to the control att IBV vaccine without IGIP.

Initial seroconversion by hemagglutination inhibition titers indicated slightly higher antibody titers in IBV att-vaccinated mice compared to the IGIP-HA-IBV att-vaccinated mice. In general, these results demonstrate that similarly to the type A IGIP-LAIV candidates, the type B IGIP-LAIV candidate virus is safe and confers protection against lethal challenge and remains a viable vaccine candidate.

In sum, the foregoing experiments illustrate strategies to produce recombinant segment 4 and segment 6 encoding chimeric HA and NA genes expressing IGIP, and successful creation of viruses of the HIN1 or H3N2 subtype carrying IGIP in either the HA or NA segments. The four chimeric segments (IGIP-H1, N1-IGIP, IGIP-H3, and N2-IGIP) viruses were rescued in the OH/04 att backbone. However, only the H1-IGIP virus was amenable for rescue in the ca/LEN backbone. Virus titers of type A IGIP recombinants in the OH/04 att backbone were reduced ˜1-2 log 10 compared to isogenic non-IGIP viruses. IGIP recombinants in the OH/04 att backbone reached titers similar to non-IGIP controls in the ca/LEN backbone. A considerably lower titer was achieved for IGIP-H1 chimera in the ca/LEN backbone compared to the non-IGIP ca/LEN control. An influenza type B IGIP-LAIV (IGIP-HA-IBV-att) with yield similar to those of the isogenic non-IGIP virus control was also engineered. Sanger and NGS sequencing of virus stocks revealed the presence of the IGIP modification as initially designed for all type A and B viruses.

The type A IGIP-LAIV (H1N1) viruses are safe in the mouse model, particularly in the IGIP-H1 att (H1N1) virus. Although the N1-IGIP att (H1N1) virus showed some virulence in female mice, IGIP modifications led to substantial attenuation compared to the att (H1N1) virus, which was not attenuated at the administered dose in DBA/2J mice. Lack of attenuation of the att (H1N1) virus is in contrast to previous studies in Balb/c and pigs. The type A IGIP-LAIV (H1N1) viruses protected mice against aggressive HIN1 challenge. A survival of 100% was observed in the vaccinated mice whereas the non-vaccinated/challenge mice succumbed between days 4 and 6 post-challenge.

The IGIP-HA-IBV-att virus was safe and efficacious against aggressive IBV challenge as 100% survival was observed in the vaccinated mice. In contrast, all male and 3/4 female non-vaccinated/challenge mice succumbed to the infection between days 5 and 8 post-challenge, respectively. Data show seroconversion and neutralizing antibody titers after boost in mice vaccinated with the IGIP-HA-IBV att virus, most likely as a consequence of active virus replication.

### Example 3: Qualitatively Different Humoral Responses are Produced by the IGIP-H1att Virus Compared to the H1caLen Virus in DBA/2J

### Mice

### Materials and Methods

Hemagglutination Inhibition Assays

Serum samples were collected at 20 dpb and 14 days post-challenge (dpc) to screen for the presence of neutralizing antibodies by hemagglutination inhibition (HAL) assays as previously described (Wan, et al., J. Virol., 92 (2018)). Briefly, the sera were treated with a receptor-destroying enzyme (Denka Seiken, VWR, PA, USA), incubated overnight at 37° C., and then inactivated at 56° C. for 30 min. After inactivation, the sera were diluted 1:10 with PBS and serially diluted 2-fold and mixed with 4 hemagglutination units (HAU) of virus in a 96-well plate. The virus/sera mixture was incubated 15 min at room temperature and the HI activity was determined after 45 min of incubation with 0.5% of turkey red blood cells (RBC). HI titers below ≤10 was arbitrarily assigned a value of 10.

Virus Neutralization Assays

The recombinant Ca/04 (HIN1) virus carrying Nano luciferase (NLuc) gene downstream PB1 was used at 100 TCID50 of per well in a 96-well plate and incubated with 1/10 serial dilutions of serum samples collected and treated as described above. The serum/virus mixture was incubated for 1 h at 37° C. and then overlayed for 15 min at 4° C. and then 45 min at 37° C. on MDCK cells seeded in a 96 well plate the day before. The serum/virus mixture was subsequently removed and 200 μL of Opti-MEM-AB+TPCK-trypsin was added, and the cells were incubated at 37° C. under 5% CO₂ for 48 h. The virus neutralization (VN) titers were visualized by classical HA assay and NLuc assay. For the NLuc luciferase assay the Nano-Glo Luciferase Assay System (Promega, Madison, WI, USA) was utilized using a Victor X3 multilabel plate reader (PerkinElmer, Waltham, MA, USA).

Influenza Antigen Microarray

The influenza antigen microarray was performed as previously described (Nakajima, et al., mSphere, 3 (2018)). Serum, BALF and NW samples were diluted 1:100 in a protein array blocking buffer (GVS, Sanford, ME, USA), supplemented with E. coli lysate (GenScript, Piscataway, NJ, USA) to a final concentration of 10 mg/mL, and preincubated at room temperature (RT) for 30 min. Concurrently, arrays were rehydrated in blocking buffer (without lysate) for 30 min. Blocking buffer was removed, and arrays were probed with preincubated serum samples using sealed chambers to prevent cross-contamination of samples between the pads. Arrays were incubated overnight at 4° C. with gentle agitation. They were then washed at RT three times with Tris-buffered saline (TBS) containing 0.05% Tween 20 (T-TBS), biotin-conjugated goat anti-mouse IgA and Biotin-conjugated anti-mouse IgG (Jackson Immuno Research Laboratories, Inc., West Grove, PA, USA) were diluted 1:400 in blocking buffer and applied to separate arrays for 1 h, RT with gentle agitation. Arrays were washed three times with T-TBS, followed by incubation with streptavidin-conjugated Qdot655 (Thermo Fisher Scientific, Waltham, MA, USA) diluted 1:200 in blocking buffer for 1 h, RT. Arrays were washed three times with T-TBS and once with water. Arrays were air dried by centrifugation at 500 g for 5 min. Images were acquired using the ArrayCAM imaging system from Grace Bio-Labs (Bend, OR, USA). Spot and background intensities were measured using an annotated grid (.gal) file. Mean fluorescence across antigens grouped by isotypes were used for subsequent analysis. The different antigens were acquired from Sino biological (Wayne, PA, USA),

### Results

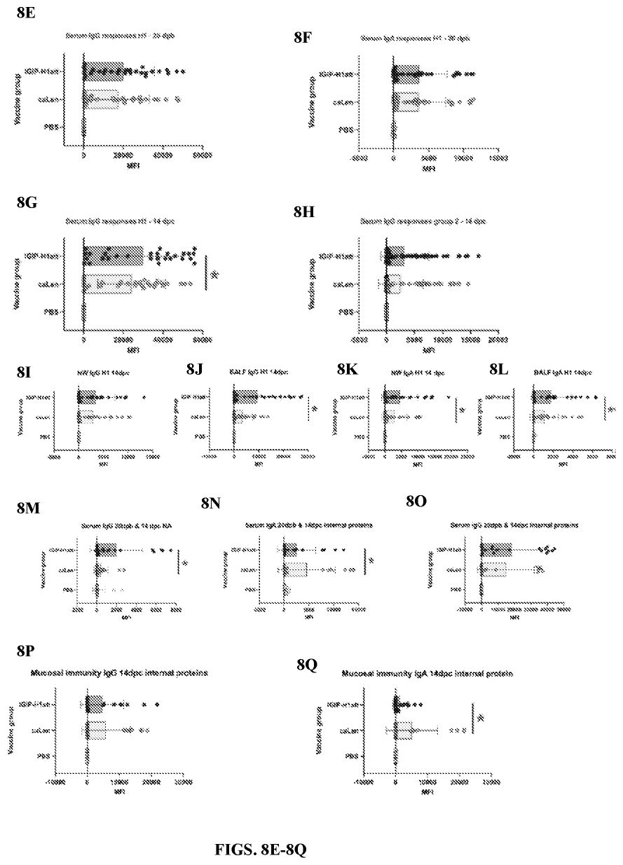

The humoral responses produced in the IGIP-H1att- and H1caLen-vaccinated mice were analyzed utilizing serum samples obtained at 20 days post-boost (20 dpb) from a subset of four mice/group (12 females) by hemagglutination inhibition (HAI) and virus neutralization (VN) titers (FIGS. 8A-8D). To establish VN titers, a recombinant Ca04 (H1N1) virus carrying a chimeric PB1 with a C-terminal Nano luciferase (Nluc) was utilized. Thus, VN titers are inversely proportional to the levels of Nluc activity measured at 48 hpi. In addition, IgG and IgA responses were analyzed using a protein microarray consisting of 153 HA proteins representing group 1 (H1, H2, H5, H6, H8, H9 and H11) and group 2 (H3, H4, H7, H10) subtypes. The protein array also contains 12 NA proteins corresponding to the N1, N2, and N9 subtypes, three M1 proteins, four NP proteins and one NS1 and one NS2 protein. Further, the array also contains 22 HA proteins and two NA proteins derived from influenza B viruses (IBVs) corresponding to the two major lineages (Victoria and Yamagata) as well as a single NP protein from a prototypic IBV, which serve as negative controls. Approximately ½ of the HA proteins are displayed as full length, whereas the rest correspond to the HAI region. Details of the strain of origin, source of the protein, and presence or absence of epitope tags are provided in the Table 5.

Both HI (average of 220 vs. 170 HI titers) and VN (average of 702 vs. 660 VN titers) titers showed a trend towards improved neutralizing responses in samples obtained from the IGIP-H1att-vaccinated mice compared to the H1caLen-vaccinated mice (FIG. 8A). This trend was consistent with a similar trend of the anti-H1 HA responses in the protein microarray, in which IGIP-H1att samples were on average higher than those from the H1caLen samples (FIGS. 8B and 8E). Average IgG responses were higher against the full HA than the HAI portion for both vaccines, perhaps due to better folding of the former and/or presence of stalk antibodies. However, it must be noted that samples from the IGIP-H1att were consistently higher against pre-pandemic HA proteins compared to the H1caLen-derived samples (statistically significantly different for the HA of A/Puerto Rico/8/34 (H1N1), p=0.045).

IgG cross reactive responses against group 1 and group 2 HAs were significantly lower compared to anti-H1 responses (FIG. 8C,8D). IgG responses to the H5 HA, particularly against the full proteins in the array showed a mixed pattern, with some reacting better with samples from the H1caLen group and some with samples from the IGIP-H1att group (FIG. 8C). Responses against H9 were close to background, except against the A/Hong Kong/35820/2009 HA antigen, in which the samples from the IGIP-H1att and H1caLen groups reacted similarly (FIG. 8D). Responses to other group 1 HAs were low, but in those well above background, a trend was observed in favor of samples from the IGIP-H1att group (FIG. 8R).

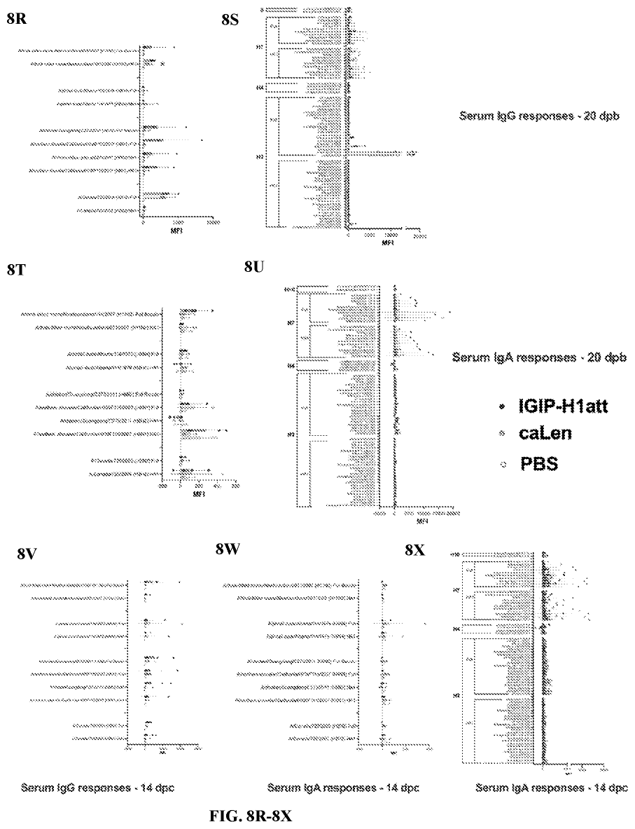

Cross-reactive responses against group 2 HAs were in general negligible, except for few full H3 antigens that were recognized similarly by serum samples from both vaccine groups, and responses to HA1 and full H7 antigens in which samples from the H1caLen group were more reactive (FIG. 8S). Both vaccine groups showed similar serum IgA response profiles against the H1 HA (9A, 8F). Background serum IgA levels against other group 1 HAs were observed except for the reactivity against the HA1 derived from A/duck/Hunan/795/2002 (H5N1) which was similar between the two vaccine groups and significantly higher than background (FIGS. 9B, 9C and 9T). Serum IgA responses against group 2 were close to background for both vaccine groups (FIG. 9U). Interestingly, some IgA serum samples from the IGIP-H1att group, but not from the H1caLen group, reacted with H7 HA antigens, which is in contrast to the IgG profile against group 2 HAs (FIG. 9U, compared to FIG. 9S).

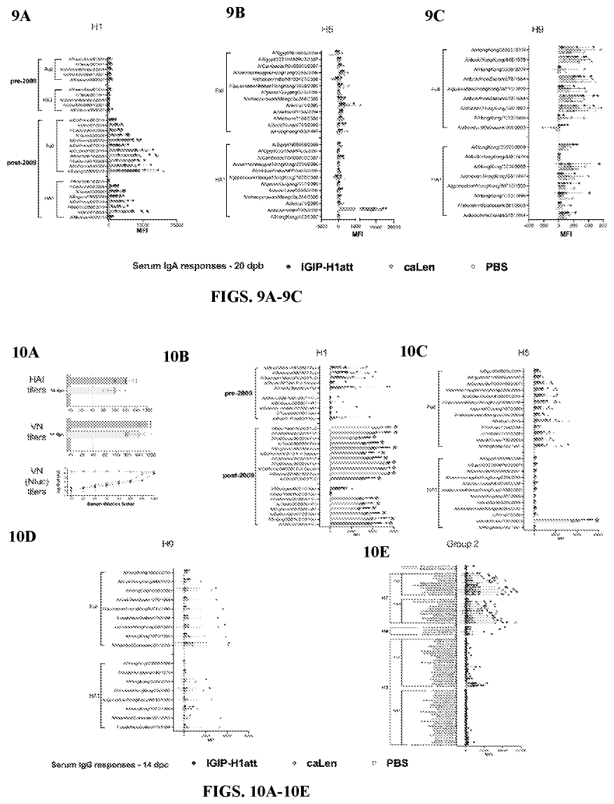

To determine whether the qualitative differences described above would translate into different recall responses post-challenge, serum samples collected at 14 dpc were analyzed. Analysis of HI and VN responses indicated about 2-fold improved responses in samples from the IGIP-H1att group compared to the H1caLen group (FIG. 10A). Analyses of IgG and IgA responses post-challenge revealed consistency with the post-boost profiles. On average, higher IgG responses were observed against the H1 HA in the IGIP-H1att serum samples than in the H1caLen serum samples collected at 14 dpc with statistically significant differences among most of the post-2009 antigens but not the pre-2009 antigens (FIG. 10B).

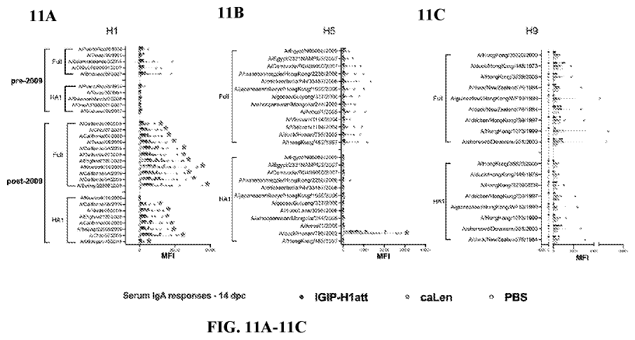

Furthermore, a significant difference between vaccines was observed when all the H1 antigens were combined (FIG. 8G). Group 1 responses showed mixed profiles, with serum samples from both vaccine groups better recognizing the full H5 than the H9 HA antigens (FIG. 10C, 10D). Neither vaccine group was particularly efficient at recalling IgG responses against other group 1 HA antigens (H2, H6, H8 and H11, FIG. 8V). Interestingly, the IGIP-H1att vaccine produced higher average IgG responses against group 2 HAs, particularly against H3 and H4, whereas responses against H7 were higher than those against H3, and both vaccine groups behaved similarly (FIGS. 10E and 8H). On average, higher serum IgA responses were observed at 14 dpc in samples from the H1caLen group compared to the IGIP-H1att group with statistically significant differences in the post-2009 H1 antigens (FIG. 11A-11C and Supplementary FIG. 8W-8X). These analyses indicated that qualitative responses to influenza viruses can be influenced by the vaccine background in mice vaccinated with different LAIVs.

### Example 4: Average Higher Anti-H1 HA Mucosal IgG and IgA Responses in the IGIP-H1att Group Compared to the H1caLen Group at 14 dpc

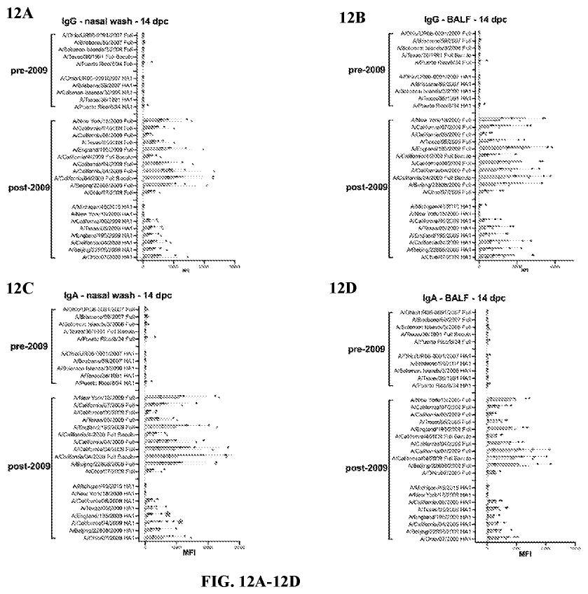

Analyses of recall mucosal responses were established using samples from nasal washes (NW; FIGS. 12A,12C) and BALF (FIGS. 12B, 12D) collected from both vaccine groups at 14 dpc. These analyses revealed a statistically significant increase in IgG and IgA in BALF and IgA in NW responses when samples from the IGIP-H1att group were compared to the H1caLen group (FIGS. 12A-12D, 81-8L). IgG and IgA responses were higher against the full H1 HA antigens than their HA1 regions. In addition, recall responses were highly focused against the 2009 H1 antigens, with little to no reactivity against pre-pandemic H1 HAs (FIGS. 12A-12D) or other group 1 and 2 HAs.

### Example 5: Humoral and Mucosal Responses Against the NA and Internal Proteins are Consistent with Anti-HA Response Patterns in the IGIP-H1att and H1caLen Groups

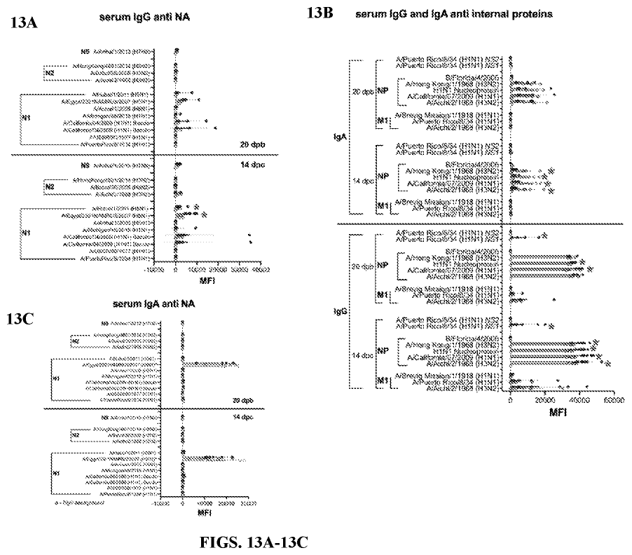

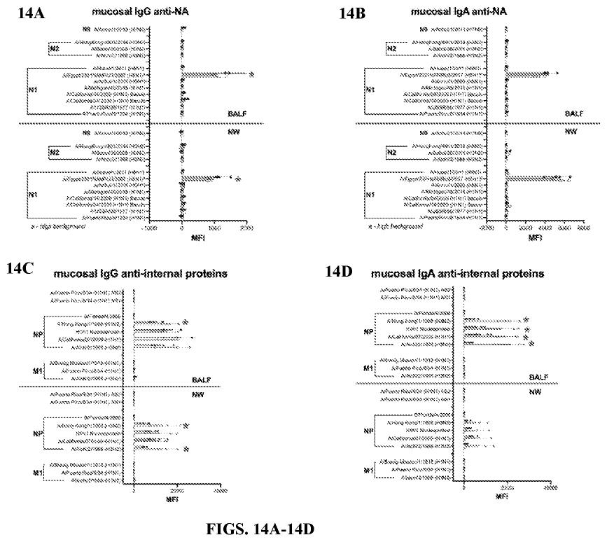

The serum and mucosal IgG and IgA profiles against the NA, NP, M1 and NS1 followed the patterns observed against HA responses (FIGS. 13A-13C and FIGS. 14A-14D). Anti-NA responses were, on average, clearly above background at 20 dpb, but only in serum samples from the IGIP-H1att group and they were largely directed to the N1 subtype (FIG. 13A), whereas those from the H1caLen had background responses. Anti-NA responses, specifically against N1, increased in the IGIP-H1att serum samples but not in those from the H1caLen group at 14 dpc and was statistically significant (FIG. 8M).

Both vaccine groups stimulated serum antibody responses against the NP, mostly IgG in the IGIP-H1att samples, but IgA in the H1caLen samples (FIG. 13B). Interestingly, only the IGIP-H1att vaccine resulted in humoral IgG responses to other internal proteins, specifically against M1 and NS1 but not NS2 (FIG. 13A-13C). M1 and NS1 responses were slightly increased after challenge in the IGIP-H1att group. Analyses of pre- and post-challenge serum responses combined indicate that antibodies against the internal proteins were dominated largely by anti-NP IgG in the IGIP-H1att group and by anti-NP IgA in the H1caLen group (FIG. 8N, 8O).

It is of note that anti-NA IgA serum responses were negligible in both vaccine groups pre- and post-challenge (FIG. 13C). Likewise, anti-NA mucosal IgG and IgA responses were at background levels for both vaccine groups (FIGS. 14A,14B). Mucosal IgG and IgA antibodies were detected against the NP but not against other internal proteins in both vaccine groups (FIGS. 14C, 14D). It is of note that the anti-NP IgG response was on average higher in NW samples obtained from the H1caLen group than that from the IGIP-H1att group, but statistically significant differences were only observed against two out of the four NP antigens evaluated. As observed with the anti-NP serological responses, they were dominated by IgG in the IGIP-H1att group (FIG. 8P) but clearly by IgA in the H1caLen group (FIG. 8Q).

Although vaccination is considered the first line of defense against influenza, the effectiveness of current IAV vaccines in recent years has been less than ideal, combined overall below 50% (Chung, et al., Clin. Infect. Dis., 71, e368-e376 (2020), Dawood, et al., MMWR Morb. Mortal. Wkly. Rep., 69, 177-182 (2020)), Rolfes, et al., Clin. Inject. Dis., 69, 1845-1853 (2019)). Although LAIVs. have the potential to provide increased multidimensional and universal cellular and humoral responses, they have also been associated with poor efficacy. In addition, one LAIV for agricultural use against swine influenza was withdrawn from the market due to safety concerns regarding reassortment with human influenza viruses. The strategies disclosed herein were tested in an effort to improve both the safety and the efficacy profiles of LAIVs. Specifically, the strategy sought to reduce the fitness of the HA segment, i.e., reduce its reassortment potential, while improving mucosal immunity against influenza. To do so, the HA segment of a prototypic 2009 HINT pandemic strain (Ca04) was modified to carry the IGIP mature peptide flanked by additional modifications and in frame with the mature HA ORF.

IGIP is highly conserved among mammals with a predicted molecular weight between ˜5.1 and ˜5.9 KDa. The IGIP mature 24 aa peptide sequence is identical in bovine, swine, and ferrets. One single amino acid difference (lysine at position 32 instead of asparagine) is seen in the human IGIP mature peptide compared to the swine IGIP. The predicted mouse IGIP differs also in one amino acid compared to the swine homolog (threonine at position 40 instead of asparagine). The role of these different polymorphisms is unknown.

In the experiments above, swine IGIP mature sequence was tested for modulation of immune responses in the context of LAIV backbones. IgA class switch in B cells occurs via both T-cell-dependent and T-cell-independent pathways, and the antibody targets both pathogenic and commensal microorganisms (Estes, Vet. Immunol. Immunopathol., 138, 312-317 (2010)). IGIP was shown to up-regulate IgA expression (Austin, et al., J. Immunol., 171, 1336-1342 (2003), Estes, Vet. Immunol. Immunopathol., 138, 312-317 (2010)). DCs in the intestinal tract are the primary source of IGIP (Estes, Vet. Immunol. Immunopathol., 138, 312-317 (2010)). The significance of DCs in the process of B cell class switch is well established (Estes, Vet. Immunol. Immunopathol., 138, 312-317 (2010)). Stimulation of human monocyte-derived DCs with CD40 L- and vasoactive intestinal peptide (VIP) leads to significant up-regulation of IGIP mRNA synthesis (˜35 fold over background). Unlike the transforming growth factor beta (TGF-β)—a well-characterized effector of B cell class switch-IGIP is not maintained in a latent form and does not require additional processing for activation (Estes, Vet. Immunol. Immunopathol., 138, 312-317 (2010)). IGIP requires the presence of CD40 ligand (CD40 L) but not B-cell receptor (BCR) cross-linking to specifically stimulate IgA class switch on bovine B cells (Austin, et at, J. Immunol., 171, 1336-1342 (2003)). In contrast, TGF-0 requires both CD40 L and BCR to exert its class switch activity on bovine B cells (Austin, et al., J. Immunol., 171, 1336-1342 (2003)). Human naïve IgD⁺ B cells can be induced towards IgA class switch and can be stimulated to produce IgA after incubation with CD40 L, IL-2, IL-10, transmembrane activator and calcium-modulator and cyclophilin ligand interactor (TACI)-Fc and either IGIP or TGF-β (Endsley, et al., J Immunol., 182, 1854-1859 (2009)). It is believed that there has been no evidence associating overexpression of IGIP with inflammatory or autoimmune diseases; however, overexpression of either APRIL, BAFF or TGF-β is associated with autoimmune diseases and cancer (Mackay, et al., Annu. Rev. Immunol., 21, 231-264 (2003), Mackay & Ambrose, Cytokine Growth Factor Rev., 14, 311-324 (2003)).

Additional modifications between the IGIP peptide and the HA ORF (G4S linker, furin cleavage site and Tav 2A protease) were introduced to help release IGIP from the mature HA and to reach the extracellular compartment. The strategy resulted in a chimeric IGIP peptide carrying a 12 aa C-terminal tail (G4S(K/R)7).

The recombinant virus IGIP-H1att grew efficiently in MDCK cells, about 1 log₁₀ lower than the isogenic H1att virus without IGIP. Moreover, the IGIP-H1att virus was stable for at least five passages in eggs. Only two mutations were identified in the HA segment of the E5 passage IGIP-H1 att virus with respect to the E1 stock virus: The first mutation L9P (t58^{non-syn}) falls within the signal peptide of the H1 HA upstream of the IGIP gene. The L9P mutation is believed (Armenteros, et al., Nat. Biotechnol., 37, 420-423 (2019)) to reduce the signal peptide cleavability from >0.9 (L9) in the wild type H1 HA sequence to 0.8765 (P9) in the mutant sequence. Nevertheless, the P9 mutation would still allow for a significant proportion of the IGIP peptide to be present without the N-terminal signal peptide sequence. The second mutation, t86c^{Syn}, corresponds to a silent mutation within the IGIP ORF, and therefore it would appear inconsequential for its potential activity. The IGIP-HA segment severely impaired the growth of the recombinant caLen vaccine virus, perhaps due to the latter containing a larger number of attenuating mutations compared to the OH/04 att backbone (Isakova-Sivak, Virology, 412, 297-305 (2011)).

Side by side comparisons were made between the IGIP-H1att, H1att, and H1caLen viruses in terms of virus growth kinetics in vitro and safety and efficacy evaluations in DBA/2 J mice. Previous studies have shown that DBA/2 J mice are 10-1,000 times more susceptible to IAV compared to C57 BL/6 and Balb/c mouse strains (Mackay & Ambrose, Cytokine Growth Factor Rev., 14, 311-324 (2003), Solorzano & Perez, J. Virol., 84, 4587-4596 (2010)). It must be noted that DBA/2 J mice are not immunodeficient and mount protective humoral responses against type A and B influenza viruses as well as other pathogens (Wan, et al., J. Virol., 92 (2018), Hollingsworth, et al., Immunogenetics, 59, 713-724 (2007), Santos, et al., J. Virol., 91 (2017)). Despite previous studies in Balb/c and pigs (Pena, et al., J. Virol., 85, 456-469 (2011), Solorzano & Perez, J. Virol., 8-, 4587-4596 (2010), Abente, et al., J. Virol., 92 (2018)) showing attenuation of different IAVs. carrying the att (ts+HA tag) modifications, such a strategy was not sufficient to attenuate the H1att virus in DBA/2 J mice. More importantly, the IGIP-H1att virus was attenuated in DBA/2 J mice as much as the control H1caLen virus. This observation also indicates that the IGIP modification leads to reduced fitness of the HA segment and therefore it will be less likely to reassort, although such assessment is beyond the scope of this report.

The IGIP-H1att virus was as efficient as the H1caLen in protecting mice against aggressive challenge with a homologous prototypic 2009 H1N1 strain. Challenge virus shedding was below the limit of detection accompanied by the absence of clinical signs in both vaccine groups. Analyses of humoral responses by different methods (HI, VN, and protein microarray) clearly showed trends of higher IgG responses in mice vaccinated with the IGIP-H1att virus, compared to those vaccinated with the H1caLen virus, not only against H1 HAs but also other group 1 HAs. Serum IgA responses post-boost were low and mostly focused to the H1 HA with similar levels between vaccine groups. In a previous study, infection of mice with a wild type H7N9 IAV led to induction of antibodies against both group 1 and group 2 HAs in the absence of discernible HAI titers (Liu, et al., J. Inject. Dis., 215, 518-528 (2017)). In this study, post-boost serum IgG responses against a panel of group 2 H7 HAs were also detected, particularly in samples from the H1caLen group. In contrast, this same H7 HA panel showed increased serum IgA reactivity using samples from the IGIP-H1att group.

At 14 dpc, the recall serum IgG antibody continued with samples from the IGIP-H1att reacting more strongly to H1 HAs than samples from the H1caLen group with statistically significant differences. Serum IgG responses to other group 1 HAs showed a mixed pattern of relatively weaker signals compared to the H1 profiles. It is of note that post-challenge resulted in boosting of group 2 HA responses, particularly against the H7 panel, but also against H3 and H4 antigens in samples from the IGIP-H1att group. In contrast, post-challenge serum IgA responses were on average statistically higher in samples from the H1caLen group. The mucosal antibody responses detected in NW and BALF at 14 dpc had overall higher average signals for both IgG and IgA in samples from the IGIP-H1att group, and statistically significant differences between vaccines for the IgG in BALF and IgA in NW and BALF.

The patterns of IgG and IgA responses against other viral proteins (N1 and NP, particularly) were consistent with those observed against HA. Serum IgG anti-N1 NA responses, as well as anti-M1 and anti-NS1, were detected above background only in samples from the IGIP-H1att group, but not in those from the H1 caLen group. In this regard, it important to note that various approaches to more universal influenza vaccines consider more conserved targets, such as epitopes on NA, M2, M1 and NP (Krammer, et al., mBio, 9 (2018), Fiers, et al., Vaccine, 27, 6280-6283 (2009), McMahon, et al., Front. Immunol., 10, 2005 (2019)). Additionally, NP modulates cellular immune response activating CD4+ and CD8+ lymphocytes providing cross-reactivity against zoonotic IAV strains (van de Sandt, et al., J. Virol., 88, 1684-1693 (2014), Lee, et al. J. Clin. Investig., 118, 3478-3490 (2008)).

In the context of LAIVs, it has been also shown that different NPs modulate differently the immune response conferring protection against heterologous challenge in the absence of neutralizing antibodies (Isakova-Sivak, et al., Vaccines, 7, 61 (2019)). FLU-v, which has shown promising results in phase II in humans, indicates that understanding the role of NP antibodies and how to modulate the NP response could pave the way for the generation of more a universal vaccine (Pleguezuelos, et al., NPJ Vaccines, 5, 22 (2020)). The disclosed results show that anti-NP responses were easily detected in serum and mucosal samples. Serum and BALF IgG dominated the response against NP in the IGIP-H1att group and in NW in the H1caLen group. Mucosal anti-NP IgA responses were on average higher in samples from the H1caLen group. It is commonly accepted that IgA responses are better at neutralizing primary viral targets such as HA, but not other viral proteins such as NP or other internal proteins (possibly even NA). In contrast, IgG responses would be better at targeting non primary targets for ADCC, complement fixation and antibody-mediated phagocytosis due to viral proteins expressed in infected cells. Thus, the pattern of IgA/IgG responses in samples from the IGIP-H1att group may indicate a superior protective advantage compared to those from the H1caLen group. Overall, these studies strongly indicate that qualitative different immunological responses can be induced in response to different LAIV backbones and subsequent modifications. It is not established whether the response patterns described above are due to IGIP exerting any biological functions. Nevertheless, it is of great significance that the IGIP modification not only improved the safety profile of the att backbone, but it did so without sacrificing immunity against the HA. Although it is accepted that IGIP is important in modulating IgA responses, such activity is considered limited to the boundaries of the intestinal tract. Little is known about IGIP function in the respiratory tract and whether it can help stimulate both IgA and IgG responses. The combined analysis of the data indicates that I.N. administration of the IGIP-H1att vaccine stimulated higher systemic IgG responses and higher IgG and IgA mucosal recall responses than the H1caLen vaccine, not only against HA but also other viral antigens. Thus IGIP may be acting as a general adjuvant in the respiratory tract that produces enhanced IgG and IgA responses.

TABLE 5
Origin and catalog number of the antigens
used in the Influenza antigen microarray†.

| | Type of | |
|---|---|---|
| Isolate | antigen | Catalog aumher |
| A/California/04/2009 (H1N1) | HA | 11055-V08B |
| A/NewYork/1 [symbol] /2009 (H1N1) | HA | 40009-V08H |
| A/England/195/2009 (H1N1) | HA | 40005-V08H |
| A/California/07/2009 (H1N1) | HA | 11085-V08H |
| A/Texas/05/2009 (H1N1) | HA | 40006-V08H |
| A/Ohio/07/2009 (H1N1) | HA | 40007-V08H |
| A/California/ [symbol] (H1N1) | HA | [symbol] |
| A/Beijing/2280 [symbol] /2009 (13IN3) | HA | 40035-V08H |
| A/Brisbane/59/2007 (H1N1) | HA | 11052-V08H |
| A/SolomonIslands/3/2006 (H1N1) | HA | 11708-V08H |
| A/Ohio/UR060991/2007 (H1N1) | HA | 11687-V08H |
| A/PuertoRico/8/1934 (H1N1) | HA | 11684-V08H |
| A/Canada/720/2005 (H2N2) | HA | 11 [symbol] 88-V08H |
| A/Hongkong/483/1997 (H5N1) | HA | 11689-V08H |
| A/goose/Guiyang/337/2006 (H5N1) | HA | [symbol] |
| A/japanesewhiteeye/HongKong/ [symbol] /2006 [symbol] | HA | 11694-V08H |
| A/chicken/India/NIV33487/2006 (H [symbol] N1) | HA | 11712-V08H |
| A/Cambodia/r0405050/2007 (H5N1) | HA | 11710-V08H |
| A/Vietnam/1194/2004 (H5N1) | HA | 11002-V08H1 |
| A/duck/Hunan/705/2002 (H5N1) | HA | 11698-V08H |
| A/whoo [symbol] /Mongolia/244/2005 | HA | 11709-V08H |
| A/Egypt/2321NAMRU3/2007 (H5N1) | HA | 11697-V08H |
| A/ [symbol] /California/HKWF115/2007 (H6N1) | HA | 11723-V08H |
| A/chicken/ [symbol] /C273/2011 (H6N2) | HA | 40398-V08B |
| A/pintailduck/Alberta/115/1979 (H8N4) | HA | 11722-V08H |
| A/shorebird/Delaware/261/2003 (H9N5) | HA | 40181-V08B |
| A/HongKong/1073/1999 (H9N2) | HA | 11229-V08H |
| A/chicken/HongKong/G9/1997 (H9N2) | HA | [symbol] -V08H |
| A/guineafowl/HongKong/WF10/1999 (H9N2) | HA | 11719-V08H |
| A/HongKong/ [symbol] /2008 (H9N2) | HA | 40178-V08B |
| A/HongKong/ [symbol] /2009 (H9N2) | HA | 40174-V08B |
| A/thickbilledmur [symbol] /Newfoundland/081/2002 (H11N2) | HA | 40187-V08B |
| A/Perth/16/2009 (H3N2) | HA | 40040- [symbol] |
| A/Hanoi/ [symbol] /2009 (H3N2) | HA | 40490-V08B |
| A/Wuhan/359/1995 (H3N2) | HA | 40475-V08B |
| A/Nanchang/ [symbol] /1995 (H3N2) | HA | 40485-V08B |
| A/Hanoi/EL [symbol] 34/2008 (H3N2) | HA | 40489-V08B |
| A/Victoria/ [symbol] /2009 (H3N2) | HA | 40058-V08B |
| A/Wisconsin/67/2005 (H3N2) | HA | 11972-V08H |
| A/Missouri/09/2014 (H3N2) | HA | 40494-V08B |
| A/Victoria/361-2011 (H3N2) | HA | 40145-V08B |
| A/California/7/2004 (H3N2) | HA | 40118-V08B |
| A/GuangdongLuohu/1256/2009 (H3N2) | HA | 40152-V08B |
| A/Victoria/208/2009 (H3N2) | HA | 40151-V08B |
| A/Texas/50/2012 (H3N2) | HA | 40354-V08B |
| A/Philippines/472/2002 (H3N2) | HA | 40437-V08B |
| A/Switzerland/9715293/2013 (H3N2) | HA | 40497-V08B |
| A/HongKong/CUHK31987/2011 (H3N2) | HA | 40146-V08B |
| A/Wyoming/03/2003 (H3N2) | HA | 11715-V08H |
| A/Netherlands/178/1995 (H3N2) | HA | 40486-V08B |
| A/Brisbane/10/2007 (H3N2) | HA | 11056-V08H |
| A/Fujian/411/2002 (H3N2) | HA | 40488-V08B |
| A/duck/Hunan/819/2009 (H4N2) | HA | 40390-V08B |
| A/mallard/Ohio/657/2002 (H4N6) | HA | 11714-V08H |
| A/Netherlands/219/2003 (H7N7) | HA | 11082-V08B |
| A/chicken/Netherlands/1/2003 (H7N7) | HA | 11212-V08B |
| A/turkey/Italy/214845/2002 (H7N3) | HA | 40125-V08B |
| A/mallard/Netherlands/33/2006 (H7N8) | HA | 40172-V08B |
| A/ [symbol] /NewJersey/563/2006 (H7N2) | HA | 40170-V08B |
| A/pigeon/Shanghai/ [symbol] /2013 (H7N9) | HA | 40106-V08H |
| A/Anhui/1/2013 (H7N9) | HA | 40103-V08H |
| A/Hangzhou/1/2013 (H7N9) | HA | 40105-V08H |
| A/Shanghai/2/2013 (H7N9) | HA | 40239-V08H |
| A/JiangxiDonghu/346/2013 (H10N8) | HA | 40359-V08B |
| A/Egypt/2321NAMEU3/2007 (H5N1) | NA | 40045-VNAHC |
| A/HongKong/4801/2014(H3N2) | NA | 40569-V07H |
| A/Aichi/2/1968 (H3N2) | NA | 40199-V07H |
| A/California/04/2009 (H1N1) | NA | 1105 [symbol] -V08B |
| A/ [symbol] /1/2011 (H5N1) | NA | [symbol] -V07H |
| A/Anhui/1/2013 (H7N9) | NA | 401 [symbol] -V07H |
| A/PuertoRico/ [symbol] /1934 (H1N1) | NA | 40196-VNAHC |
| A/Michigan/45/2015 (H1N1) | NA | 40568-V07H |
| A/Anhui/1/2005 (H5N1) | NA | 11 [symbol] 76-VNAHC |
| A/Babol/36/2005 (H3N2) | NA | 40017-V07H |
| A/USSR/90/1977 (H1N1) | NA | 40197-V07H |
| A/California/07/2009 (H1N1) | NP | 40205-V08B |
| A/Puerto Rico/8/1934 (H1N1) | NP | 11675-V08B |
| A/Hong Kong/1/1968 (H3N2; | NP | 40208-V08B |
| A/Aichi/2/1968 (H3N2) | NP | 40207-V08B |
| A/Aichi/2/1968 (H3N2) | M1 | 40215-V07E |
| A/Puerto Rico/8/1934 (H1N1) | M1 | 40010-V07E |
| A/Brevig Mission/1/1918 | M1 | 40211-V07E |
| A/Puerto Rico/8/1934 (H1N1) | N51 | 40011-V07E |
| A/Puerto Rico/8/1934 (H1N1) | N52 | 40012-VNA |

[symbol]

All antigens obtained from Sino Biological (Wayne, PA) [symbol]
indicates data missing or illegible when filed

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A nucleic acid comprising a nucleic acid sequence encoding an IgA-inducing protein (IGIP) polypeptide and a nucleic acid sequence encoding a hemagglutinin (H) or a neuraminidase (N), operably linked to one or more expression control sequences.

2. The nucleic acid of claim 1, further comprising a nucleic acid sequence encoding the autologous IGIP signal peptide sequence or a heterologous signal peptide sequence operably linked to the sequence encoding the IGIP polypeptide.

3. The nucleic acid of claim 2, wherein the IGIP polypeptide comprises the mature form of the IGIP of any one of SEQ ID NOS:1-12, or a functional fragment thereof, or variant thereof with at least 70%, 75%, 80%, 85%, 90%, 95%, or more sequence identity to any one of SEQ ID NOS:1-12, optionally wherein the IGIP polypeptide that can increase IgA expression.

4. The nucleic acid of claim 3, comprising the nucleic acid sequence encoding an H and further comprising a nucleic acid sequence encoding the autologous H signal peptide sequence or a heterologous signal peptide sequence operably linked to the sequence encoding the H.

5. The nucleic acid of claim 4, wherein the H is a mature form of an H from an influenza Type A virus, optionally wherein the H is any one of H1 to H18.

6. The nucleic acid of claim 5, wherein the H is a mature form of H1.

7. The nucleic acid of claim 4, wherein the H is a mature form of an H from an influenza Type B virus.

8. The nucleic acid of claim 3, comprising the nucleic acid sequence encoding an N and further comprising a nucleic acid sequence encoding the autologous N signal peptide sequence or a heterologous signal peptide sequence operably linked to the sequence encoding the N.

9. The nucleic acid of claim 8, wherein the N is a mature form of an N from an influenza Type A virus.

10. The nucleic acid of claim 9, wherein the N is a mature form of any one of N1 to N11.

11. The nucleic acid of claim 8, wherein the N is a mature form of an N from an influenza Type B virus.

12. The nucleic acid of claim 3, wherein the sequence encoding the IGIP polypeptide and the sequence encoding H or N are separated by a nucleic acid sequence encoding a protease cleavage site, optionally wherein the protease cleavage site is a Furin cleavage site.

13. The nucleic acid of claim 12, wherein the sequence encoding the IGIP polypeptide and the sequence encoding H or N are separated by a nucleic acid sequence encoding a self-cleaving peptide, optionally wherein the self-cleaving peptide is 2A self-cleaving peptide, optionally wherein the 2A self-cleaving peptide is selected from EGRGSLLTCGDVEENPGP (SEQ ID NO:16), ATNFSLLKQAGDVEENPGP (SEQ ID NO: 17), QCTNYALLKLAGDVESNPGP (SEQ ID NO:18), and VKQTLNFDLLKLAGDVESNPGP (SEQ ID NO:19).

14. The nucleic acid of claim 13, wherein the sequence encoding the IGIP polypeptide and the sequence encoding H or N are separated by a nucleic acid sequence encoding a peptide linker, optionally wherein the peptide linker comprises one or more glycines and one or more serines.

15. The nucleic acid of claim 3, wherein the nucleic acid is an influenza genomic RNA segment having the orientation: ‘5 nucleic acid sequence encoding IGIP polypeptide-nucleic acid sequence encoding H or N 3’.

16. The nucleic acid of claim 3, wherein the nucleic acid is an influenza genomic RNA segment having the orientation: ‘5 nucleic acid sequence encoding H or N-nucleic acid sequence encoding IGIP polypeptide 3’.

17. The nucleic acid of claim 15, further comprising 5′ untranslated region (UTR), 3′ untranslated region (UTR), or a combination thereof.

18. The nucleic acid of claim 17, wherein the 5′ UTR, 3′ UTR, or combination thereof from an influenza virus.

19. The nucleic acid of claim 18, wherein the nucleic acid encodes H and comprises the 5′ UTR and 3′ UTR of segment 4 of an influenza virus at the 5′ and 3′ ends respectively of the nucleic acid.

20. The nucleic acid of claim 18, wherein the nucleic acid encodes N and comprises the 5′ UTR and 3′ UTR of segment 6 of an influenza virus at the 5′ and 3′ ends respectively of the nucleic acid.

21. A nucleic acid comprising the reverse complementary sequence of the nucleic acid of any one of claims 1-20.

22. The nucleic acid of claim 21, wherein the nucleic acid is DNA or RNA.

23. The nucleic acid of claim 21, wherein the nucleic acid is single stranded or double stranded.

24. The nucleic acid of claim 21, wherein the nucleic acid is circular or linear.

25. A vector comprising the nucleic acid of any one of claims 1-14.

26. A viral genomic segment comprising the nucleic acid of any one of claims 1-20 or the reverse complementary sequence thereof.

27. A virus comprising the viral genomic segment of claim 26.

28. The virus of claim 27, wherein the remaining viral genomic segments are from an influenza virus.

29. The virus of claim 28, wherein the influenza virus is a Type A influenza virus.

30. The virus of claim 29, comprising the genomic structure:
Segment 1 encoding PB2;
Segment 2 encoding PB1 and optionally PB1-F2;
Segment 3 encoding PA and optionally PA-X;
Segment 4 encoding H;
Segment 5 encoding NP;
Segment 6 encoding N;
Segment 7 encoding M1 and M2; and
Segment 8 encoding NS1 and NEP.

31. The virus of claim 28, wherein the influenza virus is a Type B influenza virus.

32. The virus of claim 31, comprising the genomic structure:
Segment 1 encoding PB1;
Segment 2 encoding PB2;
Segment 3 encoding PA;
Segment 4 encoding H;
Segment 5 encoding NP;
Segment 6 encoding N and NB;
Segment 7 encoding M1 and BM2; and
Segment 8 encoding NS1 and NEP.

33. The virus of claim 27, wherein the influenza virus is an attenuated influenza virus.

34. The virus of claim 33, wherein the attenuated virus comprises a reassorted genome, temperature sensitive mutation(s), NS1 truncation, elastase-dependency, rearranged genome, or a combination thereof.

35. The virus of claim 33, wherein the attenuated influenza virus is OH/04 att, cold-adapted Leningrad (ca/LEN), or B/Bris att.

36. A pharmaceutical composition comprising the live virus of claim 27 in a pharmaceutically acceptable carrier for administration to a subject.

37. The pharmaceutical composition of claim 36, further comprising one or more additional influenza viruses.

38. The pharmaceutical composition of claim 37, wherein the one or more additional influenza viruses are attenuated viruses.

39. The pharmaceutical composition of claim 37, comprising one or more H1N1, H2N2, H3N2, H5N1, H7N7, H1N2, H9N2, H7N2, H7N3, H10N7, H7N9, and/or H6N1 influenza A subtypes.

40. The pharmaceutical composition of claim 37, comprising one or more influenza B viruses.

41. The pharmaceutical composition of claim 36, further comprising an adjuvant.

42. The pharmaceutical composition of claim 36, formulated for intradermal or intramuscular injection

43. The pharmaceutical composition of claim 36, formulated for intranasal delivery.

44. The pharmaceutical composition of claim 36, comprising an effective amount of influenza virus(es) in an effective amount to induce an immune response against influenza virus in a subject, optionally, wherein the immune response is against one or more of H, N, PB2, PB1, PA, NP, M1, M2, NS1, NEP, or a combination thereof.

45. The pharmaceutical composition of claim 44, wherein the immune response comprises an increase in production of IgA, IgG, or combination thereof antibodies against influenza virus.

46. A method of inducing or increasing an immune response against influenza virus comprising administering a subject in need thereof an effective amount of the pharmaceutical composition comprising an effective amount of a live virus comprising a viral genomic segment comprising the nucleic acid sequence of any one of claims 1-20 or the reverse complement thereof to induce or increase an immune response against the virus in the subject.

47. The method of claim 46, wherein the immune response provides a preventative or therapeutic effect against infection by influenza virus(es).

48. The method of claim 47, wherein the pharmaceutical composition is administered by injection or intranasal delivery.

49. The method of claim 48, wherein the subject is selected from humans, birds, pigs, horses, ferrets, whales, seals, dogs, cats, and rodents.

50. The method of claim 46, comprising repeating the administration one or more times 1, 2, 3, 4, 5, 6, 7, 8, 9, or 10 days, weeks, or months apart.

51. The method of claim 46, wherein the H is a mature form of an H from an influenza Type A virus.

52. The method of claim 51, wherein the H is a mature form of H1 to H18

53. The method of claim 52, wherein the H is a mature form of H1.

54. The method of claim 46, wherein the H is a mature form of an H from an influenza Type B virus.

55. The method of claim 46, comprising the nucleic acid sequence encoding an N and further comprising a nucleic acid sequence encoding the autologous N signal peptide sequence or a heterologous signal peptide sequence operably linked to the sequence encoding the N.

56. The method of claim 55, wherein the N is a mature form of an N from an influenza Type A virus.

57. The method of claim 56, wherein the N is a mature form of any one of N1 to N11.

58. The method of claim 55, wherein the N is a mature form of an N from an influenza Type B virus.

59. The method of claim 53, wherein the sequence encoding the IGIP polypeptide and the sequence encoding H are separated by a nucleic acid sequence encoding a protease cleavage site, optionally wherein the protease cleavage site is a Furin cleavage site.

60. The method of claim 54, wherein the sequence encoding the IGIP polypeptide and the sequence encoding H are separated by a nucleic acid sequence encoding a self-cleaving peptide, optionally wherein the self-cleaving peptide is 2A self-cleaving peptide, optionally wherein the 2A self-cleaving peptide is selected from EGRGSLLTCGDVEENPGP (SEQ ID NO:16), ATNFSLLKQAGDVEENPGP (SEQ ID NO:17), QCTNYALLKLAGDVESNPGP (SEQ ID NO:18), and VKQTLNFDLLKLAGDVESNPGP (SEQ ID NO:19).

61. The method of claim 60, wherein the sequence encoding the IGIP polypeptide and the sequence encoding H are separated by a nucleic acid sequence encoding a peptide linker, optionally wherein the peptide linker comprises one or more glycines and one or more serines.

62. The method of claim 53, wherein the remaining viral genomic segments are from an influenza virus.

63. The method of claim 62, wherein the influenza virus is a recombinant Type A influenza virus.

64. The method of claim 63, comprising the genomic structure:
Segment 1 encoding PB2;
Segment 2 encoding PB1 and optionally PB1-F2;
Segment 3 encoding PA and optionally PA-X;
Segment 4 encoding H;
Segment 5 encoding NP;
Segment 6 encoding N;
Segment 7 encoding M1 and M2; and
Segment 8 encoding NS1 and NEP.

65. The method of claim 62, wherein the influenza virus is a Type B influenza virus.

66. The method of claim 65, comprising the genomic structure:
Segment 1 encoding PB1;
Segment 2 encoding PB2;
Segment 3 encoding PA;
Segment 4 encoding H;
Segment 5 encoding NP;
Segment 6 encoding N and NB;
Segment 7 encoding M1 and BM2; and
Segment 8 encoding NS1 and NEP.

67. The method of claim 62, wherein the influenza virus is an attenuated influenza virus.

68. The method of claim 67, wherein the attenuated virus comprises a reassorted genome, temperature sensitive mutation(s), NS1 truncation, elastase-dependency, rearranged genome, or a combination thereof.

69. The method of claim 67, wherein the attenuated influenza virus is OH/04 att, cold-adapted Leningrad (ca/LEN), or B/Bris att.

70. The method of claim 67, wherein the immune response is against H, N, PB2, PB1, PA, NP, M1, M2, NS1, NEP, or a combination thereof.

71. A recombinant virus comprising a genome encoding an IgA-inducing protein (IGIP) polypeptide that can increase IgA and/or IgG expression when expressed in a cell infected by the virus.

72. The recombinant virus of claim 71, wherein the virus is a recombinant influenza virus, vaccinia virus, adenovirus, herpes virus, paramyxovirus 5, Newcastle disease virus, measles virus, yellow fever virus, mumps virus, rubella virus, or rotavirus.

73. A pharmaceutical composition comprising an effective amount of the recombinant virus of claims 51 or 72 to induce an immune response against the virus in a subject in need thereof, preferably wherein the immune response against the virus is greater than the immune response induced with the virus in the absence of expression of IGIP.

74. The pharmaceutical composition of claim 73, wherein the recombinant virus is a live virus.

75. A method of inducing an immune response in a subject in need thereof comprising administering the subject the pharmaceutical compositions of claim 73.

76. A polypeptide comprising the amino acid sequence of SEQ ID NO:31.


SEQUENCE LISTING (31 sequences)

SEQ ID NO: 1 - protein, 23 aa, Artificial Sequence
  synthetic polypeptide
NSPCGNQANVLCISRLEFVQYQS

SEQ ID NO: 2 - protein, 23 aa, Artificial Sequence
  synthetic polypeptide
KSPCGNQANVLCISRLEFVQYQS

SEQ ID NO: 3 - protein, 23 aa, Artificial Sequence
  synthetic polypeptide
NSPCGNQATVLCISRLEFVQYQS

SEQ ID NO: 4 - protein, 24 aa, Artificial Sequence
  synthetic polypeptide
GNSPCGNQANVLCISRLEFVQYQS

SEQ ID NO: 5 - protein, 24 aa, Artificial Sequence
  synthetic polypeptide
GKSPCGNQANVLCISRLEFVQYQS

SEQ ID NO: 6 - protein, 24 aa, Artificial Sequence
  synthetic polypeptide
GNSPCGNQATVLCISRLEFVQYQS

SEQ ID NO: 7 - protein, 47 aa, Bos taurus
MKKRSVSGCNITILAVVFSHLSAGNSPCGNQANVLCISRLEFVQYQS

SEQ ID NO: 8 - protein, 53 aa, Homo sapiens
MCSYYHMKKRSVSGCNITIFAVMFSHLSAGKSPCGNQANVLCISRLEFVQYQS

SEQ ID NO: 9 - protein, 47 aa, Artificial Sequence
  synthetic polypeptide
MKKRSVSGCNITIFAVMFSHLSAGKSPCGNQANVLCISRLEFVQYQS

SEQ ID NO: 10 - protein, 53 aa, Mus musculus
MCSYYHMKKRSVLGCNITIFAVMFSHLSAGNSPCGNQATVLCISRLEFVQYQS

SEQ ID NO: 11 - protein, 47 aa, Mus musculus
MKKRSVLGCNITIFAVMFSHLSAGNSPCGNQATVLCISRLEFVQYQS

SEQ ID NO: 12 - protein, 53 aa, Artificial Sequence
  synthetic polypeptide
MCSYYHMKKRSVSGCNITILAVMFSHLSAGNSPCGNQANVLCISRLEFVQYQS

SEQ ID NO: 13 - protein, 4 aa, Artificial Sequence
  synthetic polypeptide
  MISC_FEATURE (2)..(2): X = any amino acid
  MISC_FEATURE (3)..(3): X = Lysine (K) or Arginine (R)
RXXR

SEQ ID NO: 14 - protein, 7 aa, Artificial Sequence
  synthetic polypeptide
KRKRKKR

SEQ ID NO: 15 - protein, 8 aa, Artificial Sequence
  synthetic polypeptide
  MISC_FEATURE (2)..(2): X = any amino acid
  MISC_FEATURE (4)..(4): X = any amino acid
DXEXNPGP

SEQ ID NO: 16 - protein, 18 aa, Unknown
  Thosea asigna virus
EGRGSLLTCGDVEENPGP

SEQ ID NO: 17 - protein, 19 aa, Unknown
  porcine teschovirus-1
ATNFSLLKQAGDVEENPGP

SEQ ID NO: 18 - protein, 20 aa, Unknown
  rhinitis A virus
QCTNYALLKLAGDVESNPGP

SEQ ID NO: 19 - protein, 22 aa, Foot-and-mouth disease virus
VKQTLNFDLLKLAGDVESNPGP

SEQ ID NO: 20 - protein, 4 aa, Artificial Sequence
  synthetic polypeptide
GSGS

SEQ ID NO: 21 - protein, 4 aa, Artificial Sequence
  synthetic polypeptide
GGGS

SEQ ID NO: 22 - protein, 5 aa, Artificial Sequence
  synthetic polypeptide
GGGGS

SEQ ID NO: 23 - protein, 10 aa, Artificial Sequence
  synthetic polypeptide
GGGGSGGGGS

SEQ ID NO: 24 - protein, 20 aa, Artificial Sequence
  synthetic polypeptide
GGGGSGGGGSGGGGSGGGGS

SEQ ID NO: 25 - protein, 15 aa, Artificial Sequence
  synthetic polypeptide
GGGGSGGGGSGGGGS

SEQ ID NO: 26 - protein, 17 aa, Artificial Sequence
  synthetic polypeptide
MGVKVLFALICIAVAEA

SEQ ID NO: 27 - protein, 6 aa, Artificial Sequence
  synthetic polypeptide
CHYSEL

SEQ ID NO: 28 - protein, 74 aa, Artificial Sequence
  synthetic polypeptide
GNSPCGNQANVLCISRLEFVQYQSGGGGSKRKRKKRGLFEGRGSLLTCGDVEENPGPMGVKVLFALICIAVAEA

SEQ ID NO: 29 - protein, 69 aa, Artificial Sequence
  synthetic polypeptide
KRKRKKRGLFEGRGSLLTCGDVEENPGPMGVKVLFALICIAVAEAGNSPCGNQANVLCISRLEFVQYQS

SEQ ID NO: 30 - protein, 4 aa, Artificial Sequence
  synthetic polypeptide
  misc_feature (1)..(1): X = any amino acid
  misc_feature (3)..(3): X = any amino acid
XEXD

SEQ ID NO: 31 - protein, 36 aa, Artificial Sequence
  synthetic polypeptide
GNSPCGNQANVLCISRLEFVQYQSGGGGSKRKRKKR